(19) United States
(12) Patent Application Publication
(10) Pub. No.: US 2023/0174570 A1
(43) Pub. Date: Jun. 8, 2023

(54) NOVEL TRITERPENE DERIVATIVES AS HIV INHIBITORS

(71) Applicant: HETERO LABS LIMITED, Hyderabad (IN)

(72) Inventors: Parthasaradhi Reddy BANDI, Hyderabad (IN); Rathnakar Reddy KURA, Hyderabad (IN); Panduranga Reddy ADULLA, Hyderabad (IN); Bhaskar Reddy KASIREDDY, Hyderabad (IN)

(21) Appl. No.: 17/799,070

(22) PCT Filed: Feb. 11, 2021
(86) PCT No.: PCT/IB2021/051116

§ 371 Date: Aug. 11, 2022

(30) Foreign Application Priority Data
Feb. 11, 2020 (IN) 202041005909

(51) Int. Cl. C07J 63/00

(52) U.S. Cl. CPC C07J 63/008

(57) ABSTRACT

The present invention relates to novel triterpene derivatives of formula (I); and pharmaceutically acceptable salts thereof, wherein R₁, R₂, R₃, R₄, n, and ring

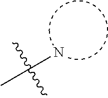 [SMILES: *N]

are as defined in formula (I). The invention also relates to novel triterpene derivatives, related compounds, and pharmaceutical compositions useful for the therapeutic treatment of viral diseases and particularly HIV mediated diseases.

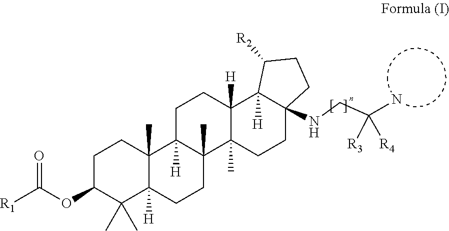

This application claims the benefit of Indian provisional application no. 202041005909 filed on 11 Feb. 2020 which is hereby incorporated by reference in its entirety.

### FIELD OF THE INVENTION

The present invention relates to novel triterpene derivatives and related compounds, compositions useful for therapeutic treatment of viral diseases and particularly HIV mediated diseases.

### BACKGROUND OF THE INVENTION

The Human Immunodeficiency Virus (HIV) has now been established as the causative agent of the Acquired Immunodeficiency Syndrome (AIDS) for over 20 years (Science 1983, 220, 868-871; N. Eng. J. Med. 1984, 311, 1292-1297). AIDS is characterized by the destruction of the immune system, particularly of CD4+T-cells. HIV is a retrovirus, and the HIV life cycle encompasses several crucial steps, starting from the attachment of the virus to the host cell membrane and finishing with the release of progeny virons from the cell.

The natural compound betulinic acid, isolated from Syzygium clavifolium and several other plant species was found to possess anti-HIV activity. Chemical modifications were undertaken by several research groups in an attempt to identify potent anti-HIV agents by making semi-synthetic analogs of betulinic acid, leading to the discovery of bevirimat as a compound with a novel mechanism of action (J. Nat. Prod. 1994, 57(2): 243-7; J. Med. Chem. 1996, 39(5), 1016). Further studies shown that bevirimat acts by disrupting Gag processing (Proc. Natl. Acad. Sci. USA 2003, 100(23):13555-60; Antimicrob. Agents. Chemother. 2001, 45(4), 1225-30; J. Virol. 2004, 78(2): 922-9; J. Biol. Chem. 2005, 280(51):42149-55; J. Virol. 2006, 80(12): 5716-22) and to be a first-in-class maturation inhibitor with a potent activity against HIV-1. Bevirimat went up to phase 2 clinical trials, in clinic despite optimal plasma concentrations, not all patients given bevirimat have a robust viral load reduction. It was reported that non-respondant patients had more frequent base line Gag polymorphisms near the capsid SP-1 cleavage site than responders. (HIV gag polymorphism determines treatment response to bevirimat. XVII international HIV drug resistance work shop Jun. 10-14, 2008, Sitges, Spain).

Encouraged by these developments, medicinal chemists started exploring betulinic acid derivatives and related compounds intensively for their therapeutic activities. For example, WO2017/149518 disclosed C-3 novel triterpene with C-17 amine derivatives as hiv inhibitors; WO2014/105926 disclosed novel betulinic acid proline derivatives as HIV inhibitors; WO2014/130810 describes preparation of C3 alkyl and alkenyl modified betulinic acid derivatives useful in the treatment of HIV; WO2014/123889 describes preparation of triterpenoid derivatives for use as HIV maturation inhibitors; WO2013/160810 disclosed novel betulinic acid derivatives as HIV inhibitors; WO2013/169578 describes C-17 bicyclic amines of triterpenoids with HIV maturation inhibitory activity and their preparation; WO 2013/123019 describes C-3 cycloalkenyl triterpenoids with HIV maturation inhibitory activity; WO2013/043778 describes novel betulinic acid derivatives with antiviral activity; WO2013/035943 describes betulinic acid and derivatives thereof having anti-aging activity; WO2012/106190 describes preparation of C17 and C3 modified triterpenoids with HIV maturation inhibitory activity; CN102399254 describes novel pentacyclic triterpenoid derivatives, their preparation method and application for preventing and treating diabetes, cardiovascular disease, cerebrovascular disease and tumor; WO2011/007230 describes preparation of lupeol-type triterpene derivatives as antiviral agents; WO2010/032123 describes preparation of triterpenoid compounds for pharmaceutical use; Journal of Medicinal Chemistry (2010), 53(1), 178-190 describes structure-activity relationship study of betulinic acid, a novel and selective TGR5 agonist, and its synthetic derivatives: potential impact in diabetes; WO 2009/100532 describes preparation of 17β-lupane derivatives for the treatment of HIV infection; CN 101367861 describes preparation method and application of 2-hydroxy-3-deoxy-pentacyclic triterpene compounds and derivatives; WO2008/138200 describes preparation of lupane derivatives as NMDA and MC receptor antagonists exhibiting neuroprotective and memory enhancing activities; WO2008/127364 describes preparation of betulinic acid derivatives for use in antiviral and anticancer pharmaceutical compositions.

Some additional references disclose betulinic acid related compounds. For example, WO2006/053255 describes preparation of betulin derivatives for use in pharmaceutical compositions which inhibit the transmission of viral infection; WO2004/089357 describes anti-fungal formulation of triterpene and essential oil; Bioorganic & Medicinal Chemistry Letters (2003), 13(20), 3549-3552 describes lupane triterpenes and derivatives with antiviral activity; Russian Journal of Bioorganic Chemistry (2003), 29(6), 594-600 describes synthesis and antiviral activity of ureides and carbamates of betulinic acid and its derivatives; Journal of Medicinal Chemistry (1996), 39(5), 1056-68 describes betulinic acid derivatives: a new class of human immunodeficiency virus type 1 specific inhibitors with a new mode of action; Oxidation Communications (1987), 10(3-4), 305-12 describes oxidative decarboxylations. II. oxidative decarboxylation of acetyl betulinic acid.

Given the fact of the world wide epidemic level of AIDS, there is a strong continued need for new effective drugs for treatment of HIV infected patients, disease conditions and/or disorders mediated by HIV by discovering new compounds with novel structures and/or mechanism of action(s).

### SUMMARY OF THE INVENTION

The present invention relates to the compound of the formula (I):

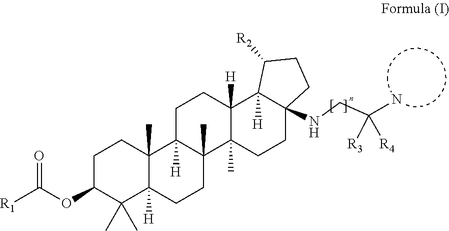

wherein, R₁ is selected from

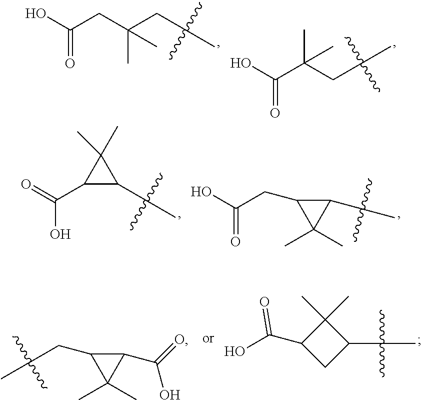

R₂ is selected from C₁-C₆ alkyl, optionally substituted C₂-C₆ alkenyl, or optionally substituted C₃-C₈ cycloalkyl; wherein the optional substituent is C₁-C₆ alkyl;

R₃ is hydrogen, or C₁-C₆ alkyl;

R₄ is selected from optionally substituted C₁-C₆ alkyl, or —C(O)ORₐ; wherein the optional substituent is selected from halo, hydroxy, alkoxy, —OC(O)CH₂alkoxy, —OSi(Rₐ)₃, —N(Rₐ)(R_{b}), —S(Rₐ), or —O—CH₂—P(O)(ORₐ)₂;

ring

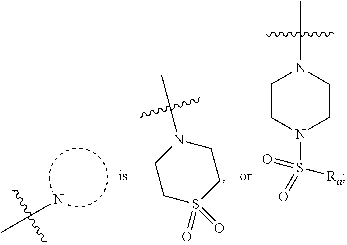

Rₐ is hydrogen, or C₁-C₆ alkyl;

R_{b} is selected from hydrogen, alkyl, —C(O)alkoxy, or —S(O)₂alkyl, and

‘n’ is an integer selected from 1-2; or

pharmaceutically acceptable salts, pharmaceutically acceptable stereoisomers, prodrugs, or combination thereof.

According to one embodiment, there is provided a compound of formula (I), wherein R₁ is

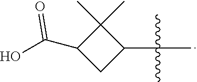 [SMILES: *C1CC(C(=O)O)C1(C)C]

According to another embodiment, there is provided a compound of formula (I), wherein R₁ is

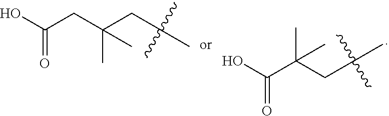

According to yet another embodiment, there is provided a compound of formula (I), wherein R₂ is isopropylene.

According to yet another embodiment, there is provided a compound of formula (I), wherein R₂ is methylcyclopropyl.

According to yet another embodiment, there is provided a compound of formula (I), wherein R₃ is hydrogen.

According to yet another embodiment, there is provided a compound of formula (I), wherein ‘n’ is 1 or 2.

According to yet another embodiment, there is provided a compound of formula (I), wherein ring

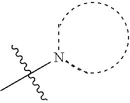

is

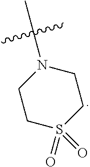 [SMILES: *N1CCS(=O)(=O)CC1]

Accordingly, another aspect of the present invention provides compound of formula (IA):

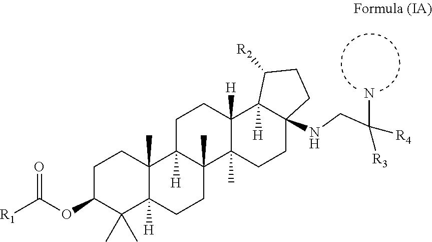

wherein R₁, R₂, R₃, R₄, and ring

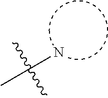 [SMILES: *N]

are same as defined in formula (I); or

pharmaceutically acceptable salts, pharmaceutically acceptable stereoisomers, prodrugs, or combination thereof.

According to one embodiment, there is provided a compound of formula (IA), wherein R₂ is isopropylene.

According to another embodiment, there is provided a compound of formula (IA), wherein R₂ is methylcyclopropyl.

According to yet another embodiment there is provided a compound of formula (IA), wherein R₃ is hydrogen.

According to yet another embodiment there is provided a compound of formula (IA), wherein R₃ is methyl.

According to yet another embodiment, there is provided a compound of formula (I), wherein ‘n’ is 2.

According to yet another embodiment, there is provided a compound of formula (IA), wherein ring

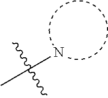 [SMILES: *N]

is

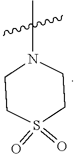 [SMILES: *N1CCS(=O)(=O)CC1]

Accordingly, another aspect of the present invention provides compound of formula (IB):

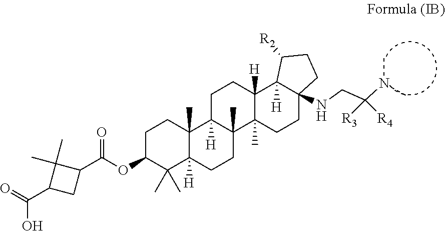

wherein R₂, R₃, R₄, and ring

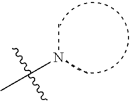

are same as defined in formula (I); or

pharmaceutically acceptable salts, pharmaceutically acceptable stereoisomers, prodrugs, or combination thereof.

According to one embodiment, there is provided a compound of formula (IA), wherein R₂ is isopropylene.

According to another embodiment, there is provided a compound of formula (IA), wherein R₂ is methylcyclopropyl.

According to yet another embodiment there is provided a compound of formula (IA), wherein R₃ is hydrogen.

According to yet another embodiment there is provided a compound of formula (IA), wherein R₃ is methyl.

According to yet another embodiment, there is provided a compound of formula (I), wherein ‘n’ is 2.

According to yet another embodiment, there is provided a compound of formula (IA), wherein ring

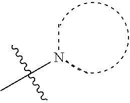

is

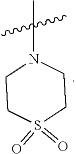 [SMILES: *N1CCS(=O)(=O)CC1]

Below are the representative compounds, which are illustrative in nature only and are not intended to limit to the scope of the invention (Nomenclature has been generated from ChemBioDraw Ultra 13.0 version):
(1R,3S)-3-((((1R,3aS,5aR,5bR,7aR,9S,11aR,11bR,13aR,13bR)-3a-(((R)-2-(1,1-dioxidothiomorpholino)-3-methoxy-3-oxopropyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
5-(((1R,3aS,5aR,5bR,7aR,9S,11aR,11bR,13aR,13bR)-3a-(((2R,3R)-2-(1,1-dioxidothiomorpholino)-3-hydroxybutyl)amino)-5a,5b,8,8,11a-pentamethyl-1-(prop-1-en-2-yl)icosahydro-1H-cyclopenta[a]chrysen-9-yl)oxy)-3,3-dimethyl-5-oxopentanoic acid,
(1R,3S)-3-((((1R,3aS,5aR,5bR,7aR,9S,11aR,11bR,13aR,13bR)-3a-(((2S,3S)-2-(1,1-dioxidothiomorpholino)-3-hydroxybutyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((2R,3S)-2-(1,1-dioxidothiomorpholino)-3-hydroxybutyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((2S,3R)-2-(1,1-dioxidothiomorpholino)-3-hydroxybutyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((2R,3S)-2-(1,1-dioxidothiomorpholino)-3-hydroxybutyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((2S,3R)-2-(1,1-dioxidothiomorpholino)-3-hydroxybutyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1R,3S)-3-((((1S,3aS,5aR,5bR,7aR,9S,11aR,11bR,13aR,13bR)-3a-(((2R,3R)-2-(1,1-dioxidothiomorpholino)-3-hydroxybutyl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1R,3S)-3-((((1S,3aS,5aR,5bR,7aR,9S,11aR,11bR,13aR,13bR)-3a-(((2S,3S)-2-(1,1-dioxidothiomorpholino)-3-hydroxybutyl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1R,3S)-3-((((1S,3aS,5aR,5bR,7aR,9S,11aR,11bR,13aR,13bR)-3a-(((2R,3S)-2-(1,1-dioxidothiomorpholino)-3-hydroxybutyl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1R,3S)-3-((((1S,3aS,5aR,5bR,7aR,9S,11aR,11bR,13aR,13bR)-3a-(((2S,3R)-2-(1,1-dioxidothiomorpholino)-3-hydroxybutyl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1S,3R)-3-((((1R,3aS,5aR,5bR,7aR,9S,11aR,11bR,13aR,13bR)-3a-(((2R,3R)-2-(1,1-dioxidothiomorpholino)-3-hydroxybutyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1S,3R)-3-((((1R,3aS,5aR,5bR,7aR,9S,11aR,11bR,13aR,13bR)-3a-(((2S,3S)-2-(1,1-dioxidothiomorpholino)-3-hydroxybutyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1S,3R)-3-((((1R,3aS,5aR,5bR,7aR,9S,11aR,11bR,13aR,13bR)-3a-(((2R,3S)-2-(1,1-dioxidothiomorpholino)-3-hydroxybutyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1S,3R)-3-((((1R,3aS,5aR,5bR,7aR,9S,11aR,11bR,13aR,13bR)-3a-(((2S,3R)-2-(1,1-dioxidothiomorpholino)-3-hydroxybutyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1S,3R)-3-((((1R,3aS,5aR,5bR,7aR,9S,11aR,11bR,13aR,13bR)-3a-(((2R,3R)-2-(1,1-dioxidothiomorpholino)-3-hydroxybutyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1S,3R)-3-((((1R,3aS,5aR,5bR,7aR,9S,11aR,11bR,13aR,13bR)-3a-(((2S,3S)-2-(1,1-dioxidothiomorpholino)-3-hydroxybutyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1S,3R)-3-((((1R,3aS,5aR,5bR,7aR,9S,11aR,11bR,13aR,13bR)-3a-(((2R,3S)-2-(1,1-dioxidothiomorpholino)-3-hydroxybutyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1S,3R)-3-((((1R,3aS,5aR,5bR,7aR,9S,11aR,11bR,13aR,13bR)-3a-(((2S,3R)-2-(1,1-dioxidothiomorpholino)-3-hydroxybutyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1S,3R)-3-((((1S,3aS,5aR,5bR,7aR,9S,11aR,11bR,13aR,13bR)-3a-(((2R,3R)-2-(1,1-dioxidothiomorpholino)-3-hydroxybutyl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1S,3R)-3-((((1S,3aS,5aR,5bR,7aR,9S,11aR,11bR,13aR,13bR)-3a-(((2S,3S)-2-(1,1-dioxidothiomorpholino)-3-hydroxybutyl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1S,3R)-3-((((1S,3aS,5aR,5bR,7aR,9S,11aR,11bR,13aR,13bR)-3a-(((2R,3S)-2-(1,1-dioxidothiomorpholino)-3-hydroxybutyl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1S,3R)-3-((((1S,3aS,5aR,5bR,7aR,9S,11aR,11bR,13aR,13bR)-3a-(((2S,3R)-2-(1,1-dioxidothiomorpholino)-3-hydroxybutyl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((2R,3R)-2-(1,1-dioxidothiomorpholino)-3-hydroxybutyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclopropane-1-carboxylic acid,
(1S,3R)-3-((((1R,3aS,5aR,5bR,7aR,9S,11aR,11bR,13aR,13bR)-3a-(((2R,3R)-2-(1,1-dioxidothiomorpholino)-3-hydroxybutyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclopropane-1-carboxylic acid,
(1R,3S)-3-((((1R,3aS,5aR,5bR,7aR,9S,11aR,11bR,13aR,13bR)-3a-(((2S,3S)-2-(1,1-dioxidothiomorpholino)-3-hydroxybutyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclopropane-1-carboxylic acid,
(1S,3R)-3-((((1R,3aS,5aR,5bR,7aR,9S,11aR,11bR,13aR,13bR)-3a-(((2S,3S)-2-(1,1-dioxidothiomorpholino)-3-hydroxybutyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclopropane-1-carboxylic acid,
(1S,3R)-3-(2-(((1R,3aS,5aR,5bR,7aR,9S,11aR,11bR,13aR,13bR)-3a-(((2R,3R)-2-(1,1-dioxidothiomorpholino)-3-hydroxybutyl)amino)-5a,5b,8,8,11a-pentamethyl-1-(prop-1-en-2-yl)icosahydro-1H-cyclopenta[a]chrysen-9-yl)oxy)-2-oxoethyl)-2,2-dimethylcyclopropane-1-carboxylic acid,
(1S,3R)-3-(2-(((1R,3aS,5aR,5bR,7aR,9S,11aR,11bR,13aR,13bR)-3a-(((2S,3S)-2-(1,1-dioxidothiomorpholino)-3-hydroxybutyl)amino)-5a,5b,8,8,11a-pentamethyl-1-(prop-1-en-2-yl)icosahydro-1H-cyclopenta[a]chrysen-9-yl)oxy)-2-oxoethyl)-2,2-dimethylcyclopropane-1-carboxylic acid,
2-((1R,3S)-3-((((1R,3aS,5aR,5bR,7aR,9S,11aR,11bR,13aR,13bR)-3a-(((2R,3R)-2-(1,1-dioxidothiomorpholino)-3-hydroxybutyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclopropyl)acetic acid,
2-((1R,3S)-3-((((1R,3aS,5aR,5bR,7aR,9S,11aR,11bR,13aR,13bR)-3a-(((2S,3S)-2-(1,1-dioxidothiomorpholino)-3-hydroxybutyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclopropyl)acetic acid,
2-((1R,3S)-3-((((1R,3aS,5aR,5bR,7aR,9S,11aR,11bR,13aR,13bR)-3a-(((2R,3R)-2-(1,1-dioxidothiomorpholino)-3-hydroxybutyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclopropyl)acetic acid,
2-((1R,3S)-3-((((1R,3aS,5aR,5bR,7aR,9S,11aR,11bR,13aR,13bR)-3a-(((2S,3S)-2-(1,1-dioxidothiomorpholino)-3-hydroxybutyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclopropyl)acetic acid,
2-((1R,3S)-3-((((1S,3aS,5aR,5bR,7aR,9S,11aR,11bR,13aR,13bR)-3a-(((2R,3R)-2-(1,1-dioxidothiomorpholino)-3-hydroxybutyl)amino)-1-isopropyl-5a,5b,8,8,11a-pentamethylicosahydro-1H-cyclopenta[a]chrysen-9-yl)oxy)carbonyl)-2,2-dimethylcyclopropyl)acetic acid,
2-((1R,3S)-3-((((1S,3aS,5aR,5bR,7aR,9S,11aR,11bR,13aR,13bR)-3a-(((2S,3S)-2-(1,1-dioxidothiomorpholino)-3-hydroxybutyl)amino)-1-isopropyl-5a,5b,8,8,11a-pentamethylicosahydro-1H-cyclopenta[a]chrysen-9-yl)oxy)carbonyl)-2,2-dimethylcyclopropyl)acetic acid,
4-(((1R,3aS,5aR,5bR,7aR,9S,11aR,11bR,13aR,13bR)-3a-(((2R,3R)-2-(1,1-dioxidothiomorpholino)-3-hydroxybutyl)amino)-5a,5b,8,8,11a-pentamethyl-1-(prop-1-en-2-yl)icosahydro-1H-cyclopenta[a]chrysen-9-yl)oxy)-2,2-dimethyl-4-oxobutanoic acid,
4-(((1R,3aS,5aR,5bR,7aR,9S,11aR,11bR,13aR,13bR)-3a-(((2S,3S)-2-(1,1-dioxidothiomorpholino)-3-hydroxybutyl)amino)-5a,5b,8,8,11a-pentamethyl-1-(prop-1-en-2-yl)icosahydro-1H-cyclopenta[a]chrysen-9-yl)oxy)-2,2-dimethyl-4-oxobutanoic acid,
4-(((1R,3aS,5aR,5bR,7aR,9S,11aR,11bR,13aR,13bR)-3a-(((2R,3R)-2-(1,1-dioxidothiomorpholino)-3-hydroxybutyl)amino)-5a,5b,8,8,11a-pentamethyl-1-(1-methylcyclopropyl)icosahydro-1H-cyclopenta[a]chrysen-9-yl)oxy)-2,2-dimethyl-4-oxobutanoic acid,
4-(((1R,3aS,5aR,5bR,7aR,9S,11aR,11bR,13aR,13bR)-3a-(((2S,3S)-2-(1,1-dioxidothiomorpholino)-3-hydroxybutyl)amino)-5a,5b,8,8,11a-pentamethyl-1-(1-methylcyclopropyl)icosahydro-1H-cyclopenta[a]chrysen-9-yl)oxy)-2,2-dimethyl-4-oxobutanoic acid,
4-(((1S,3aS,5aR,5bR,7aR,9S,11aR,11bR,13aR,13bR)-3a-(((2R,3R)-2-(1,1-dioxidothiomorpholino)-3-hydroxybutyl)amino)-1-isopropyl-5a,5b,8,8,11a-pentamethylicosahydro-1H-cyclopenta[a]chrysen-9-yl)oxy)-2,2-dimethyl-4-oxobutanoic acid,
4-(((1S,3aS,5aR,5bR,7aR,9S,11aR,11bR,13aR,13bR)-3a-(((2S,3S)-2-(1,1-dioxidothiomorpholino)-3-hydroxybutyl)amino)-1-isopropyl-5a,5b,8,8,11a-pentamethylicosahydro-1H-cyclopenta[a]chrysen-9-yl)oxy)-2,2-dimethyl-4-oxobutanoic acid,
5-(((1R,3aS,5aR,5bR,7aR,9S,11aR,11bR,13aR,13bR)-3a-(((2R,3R)-2-(1,1-dioxidothiomorpholino)-3-hydroxybutyl)amino)-5a,5b,8,8,11a-pentamethyl-1-(prop-1-en-2-yl)icosahydro-1H-cyclopenta[a]chrysen-9-yl)oxy)-3,3-dimethyl-5-oxopentanoic acid,
5-(((1R,3aS,5aR,5bR,7aR,9S,11aR,11bR,13aR,13bR)-3a-(((2S,3S)-2-(1,1-dioxidothiomorpholino)-3-hydroxybutyl)amino)-5a,5b,8,8,11a-pentamethyl-1-(prop-1-en-2-yl)icosahydro-1H-cyclopenta[a]chrysen-9-yl)oxy)-3,3-dimethyl-5-oxopentanoic acid,
5-(((1R,3aS,5aR,5bR,7aR,9S,11aR,11bR,13aR,13bR)-3a-(((2R,3R)-2-(1,1-dioxidothiomorpholino)-3-hydroxybutyl)amino)-5a,5b,8,8,11a-pentamethyl-1-(1-methylcyclopropyl)icosahydro-1H-cyclopenta[a]chrysen-9-yl)oxy)-3,3-dimethyl-5-oxopentanoic acid,
5-(((1R,3aS,5aR,5bR,7aR,9S,11aR,11bR,13aR,13bR)-3a-(((2S,3S)-2-(1,1-dioxidothiomorpholino)-3-hydroxybutyl)amino)-5a,5b,8,8,11a-pentamethyl-1-(1-methylcyclopropyl)icosahydro-1H-cyclopenta[a]chrysen-9-yl)oxy)-3,3-dimethyl-5-oxopentanoic acid,
5-(((1S,3aS,5aR,5bR,7aR,9S,11aR,11bR,13aR,13bR)-3a-(((2R,3R)-2-(1,1-dioxidothiomorpholino)-3-hydroxybutyl)amino)-1-isopropyl-5a,5b,8,8,11a-pentamethylicosahydro-1H-cyclopenta[a]chrysen-9-yl)oxy)-3,3-dimethyl-5-oxopentanoic acid,
5-(((1S,3aS,5aR,5bR,7aR,9S,11aR,11bR,13aR,13bR)-3a-(((2S,3S)-2-(1,1-dioxidothiomorpholino)-3-hydroxybutyl)amino)-1-isopropyl-5a,5b,8,8,11a-pentamethylicosahydro-1H-cyclopenta[a]chrysen-9-yl)oxy)-3,3-dimethyl-5-oxopentanoic acid,
4-(((1R,3aS,5aR,5bR,7aR,9S,11aR,11bR,13aR,13bR)-3a-(((2R,3R)-3-((dimethoxyphosphoryl)methoxy)-2-(1,1-dioxidothiomorpholino)butyl)amino)-5a,5b,8,8,11a-pentamethyl-1-(prop-1-en-2-yl)icosahydro-1H-cyclopenta[a]chrysen-9-yl)oxy)-2,2-dimethyl-4-oxobutanoic acid,
4-(((1R,3aS,5aR,5bR,7aR,9S,11aR,11bR,13aR,13bR)-3a-(((2R,3R)-2-(1,1-dioxidothiomorpholino)-3-(phosphonomethoxy)butyl)amino)-5a,5b,8,8,11a-pentamethyl-1-(prop-1-en-2-yl)icosahydro-1H-cyclopenta[a]chrysen-9-yl)oxy)-2,2-dimethyl-4-oxobutanoic acid,
(1R,3S)-3-((((1R,3aS,5aR,5bR,7aR,9S,11aR,11bR,13aR,13bR)-3a-(((2R,3R)-3-((dimethoxyphosphoryl)methoxy)-2-(1,1-dioxidothiomorpholino)butyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((2R,3R)-2-(1,1-dioxidothiomorpholino)-3-(phosphonomethoxy)butyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((2R,3R)-3-((dimethoxyphosphoryl)methoxy)-2-(1,1-dioxidothiomorpholino)butyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((2R,3R)-2-(1,1-dioxidothiomorpholino)-3-(phosphonomethoxy)butyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1R,3S)-3-((((1S,3aS,5aR,5bR,7aR,9S,11aR,11bR,13aR,13bR)-3a-(((2R,3R)-3-((dimethoxyphosphoryl)methoxy)-2-(1,1-dioxidothiomorpholino)butyl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1R,3S)-3-((((1S,3aS,5aR,5bR,7aR,9S,11aR,11bR,13aR,13bR)-3a-(((2R,3R)-2-(1,1-dioxidothiomorpholino)-3-(phosphonomethoxy)butyl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
5-(((1R,3aS,5aR,5bR,7aR,9S,11aR,11bR,13aR,13bR)-3a-(((2R,3R)-3-((dimethoxyphosphoryl)methoxy)-2-(1,1-dioxidothiomorpholino)butyl)amino)-5a,5b,8,8,11a-pentamethyl-1-(prop-1-en-2-yl)icosahydro-1H-cyclopenta[a]chrysen-9-yl)oxy)-3,3-dimethyl-5-oxopentanoic acid,
5-(((1R,3aS,5aR,5bR,7aR,9S,11aR,11bR,13aR,13bR)-3a-(((2R,3R)-2-(1,1-dioxidothiomorpholino)-3-(phosphonomethoxy)butyl)amino)-5a,5b,8,8,11a-pentamethyl-1-(prop-1-en-2-yl)icosahydro-1H-cyclopenta[a]chrysen-9-yl)oxy)-3,3-dimethyl-5-oxopentanoic acid,
5-(((1R,3aS,5aR,5bR,7aR,9S,11aR,11bR,13aR,13bR)-3a-(((2R,3R)-3-((dimethoxyphosphoryl)methoxy)-2-(1,1-dioxidothiomorpholino)butyl)amino)-5a,5b,8,8,11a-pentamethyl-1-(1-methylcyclopropyl)icosahydro-1H-cyclopenta[a]chrysen-9-yl)oxy)-3,3-dimethyl-5-oxopentanoic acid,
5-(((1R,3aS,5aR,5bR,7aR,9S,11aR,11bR,13aR,13bR)-3a-(((2R,3R)-2-(1,1-dioxidothiomorpholino)-3-(phosphonomethoxy)butyl)amino)-5a,5b,8,8,11a-pentamethyl-1-(1-methylcyclopropyl)icosahydro-1H-cyclopenta[a]chrysen-9-yl)oxy)-3,3-dimethyl-5-oxopentanoic acid,
5-(((1S,3aS,5aR,5bR,7aR,9S,11aR,11bR,13aR,13bR)-3a-(((2R,3R)-3-((dimethoxyphosphoryl)methoxy)-2-(1,1-dioxidothiomorpholino)butyl)amino)-1-isopropyl-5a,5b,8,8,11a-pentamethylicosahydro-1H-cyclopenta[a]chrysen-9-yl)oxy)-3,3-dimethyl-5-oxopentanoic acid,
5-(((1S,3aS,5aR,5bR,7aR,9S,11aR,11bR,13aR,13bR)-3a-(((2R,3R)-2-(1,1-dioxidothiomorpholino)-3-(phosphonomethoxy)butyl)amino)-1-isopropyl-5a,5b,8,8,11a-pentamethylicosahydro-1H-cyclopenta[a]chrysen-9-yl)oxy)-3,3-dimethyl-5-oxopentanoic acid,
2-((1R,3S)-3-((((1R,3aS,5aR,5bR,7aR,9S,11aR,11bR,13aR,13bR)-3a-(((2R,3R)-3-((dimethoxyphosphoryl)methoxy)-2-(1,1-dioxidothiomorpholino)butyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclopropyl)acetic acid,
2-((1R,3S)-3-((((1R,3aS,5aR,5bR,7aR,9S,11aR,11bR,13aR,13bR)-3a-(((2R,3R)-2-(1,1-dioxidothiomorpholino)-3-(phosphonomethoxy)butyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclopropyl)acetic acid,
2-((1R,3S)-3-((((1R,3aS,5aR,5bR,7aR,9S,11aR,11bR,13aR,13bR)-3a-(((2R,3R)-3-((dimethoxyphosphoryl)methoxy)-2-(1,1-dioxidothiomorpholino)butyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclopropyl)acetic acid,
2-((1R,3S)-3-((((1R,3aS,5aR,5bR,7aR,9S,11aR,11bR,13aR,13bR)-3a-(((2R,3R)-2-(1,1-dioxidothiomorpholino)-3-(phosphonomethoxy)butyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclopropyl)acetic acid,
2-((1R,3S)-3-((((1S,3aS,5aR,5bR,7aR,9S,11aR,11bR,13aR,13bR)-3a-(((2R,3R)-3-((dimethoxyphosphoryl)methoxy)-2-(1,1-dioxidothiomorpholino)butyl)amino)-1-isopropyl-5a,5b,8,8,11a-pentamethylicosahydro-1H-cyclopenta[a]chrysen-9-yl)oxy)carbonyl)-2,2-dimethylcyclopropyl)acetic acid,
2-((1R,3S)-3-((((1S,3aS,5aR,5bR,7aR,9S,11aR,11bR,13aR,13bR)-3a-(((2R,3R)-2-(1,1-dioxidothiomorpholino)-3-(phosphonomethoxy)butyl)amino)-1-isopropyl-5a,5b,8,8,11a-pentamethylicosahydro-1H-cyclopenta[a]chrysen-9-yl)oxy)carbonyl)-2,2-dimethylcyclopropyl)acetic acid,
4-(((1R,3aS,5aR,5bR,7aR,9S,11aR,11bR,13aR,13bR)-3a-(((12R,13R)-13-(1,1-dioxidothiomorpholino)-12-methyl-2,5,8,11-tetraoxatetradecan-14-yl)amino)-5a,5b,8,8,11a-pentamethyl-1-(prop-1-en-2-yl)icosahydro-1H-cyclopenta[a]chrysen-9-yl)oxy)-2,2-dimethyl-4-oxobutanoic acid,
4-(((1R,3aS,5aR,5bR,7aR,9S,11aR,11bR,13aR,13bR)-3a-(((12R,13R)-13-(1,1-dioxidothiomorpholino)-12-methyl-2,5,8,11-tetraoxatetradecan-14-yl)amino)-5a,5b,8,8,11a-pentamethyl-1-(1-methylcyclopropyl)icosahydro-1H-cyclopenta[a]chrysen-9-yl)oxy)-2,2-dimethyl-4-oxobutanoic acid,
4-(((1S,3aS,5aR,5bR,7aR,9S,11aR,11bR,13aR,13bR)-3a-(((12R,13R)-13-(1,1-dioxidothiomorpholino)-12-methyl-2,5,8,11-tetraoxatetradecan-14-yl)amino)-1-isopropyl-5a,5b,8,8,11a-pentamethylicosahydro-1H-cyclopenta[a]chrysen-9-yl)oxy)-2,2-dimethyl-4-oxobutanoic acid,
5-(((1R,3aS,5aR,5bR,7aR,9S,11aR,11bR,13aR,13bR)-3a-(((12R,13R)-13-(1,1-dioxidothiomorpholino)-12-methyl-2,5,8,11-tetraoxatetradecan-14-yl)amino)-5a,5b,8,8,11a-pentamethyl-1-(prop-1-en-2-yl)icosahydro-1H-cyclopenta[a]chrysen-9-yl)oxy)-3,3-dimethyl-5-oxopentanoic acid,
5-(((1R,3aS,5aR,5bR,7aR,9S,11aR,11bR,13aR,13bR)-3a-(((12R,13R)-13-(1,1-dioxidothiomorpholino)-12-methyl-2,5,8,11-tetraoxatetradecan-14-yl)amino)-5a,5b,8,8,11a-pentamethyl-1-(1-methylcyclopropyl)icosahydro-1H-cyclopenta[a]chrysen-9-yl)oxy)-3,3-dimethyl-5-oxopentanoic acid,
5-(((1S,3aS,5aR,5bR,7aR,9S,11aR,11bR,13aR,13bR)-3a-(((12R,13R)-13-(1,1-dioxidothiomorpholino)-12-methyl-2,5,8,11-tetraoxatetradecan-14-yl)amino)-1-isopropyl-5a,5b,8,8,11a-pentamethylicosahydro-1H-cyclopenta[a]chrysen-9-yl)oxy)-3,3-dimethyl-5-oxopentanoic acid,
4-(((1R,3aS,5aR,5bR,7aR,9S,11aR,11bR,13aR,13bR)-3a-(((15R,16R)-16-(1,1-dioxidothiomorpholino)-15-methyl-13-oxo-2,5,8,11,14-pentaoxaheptadecan-17-yl)amino)-5a,5b,8,8,11a-pentamethyl-1-(prop-1-en-2-yl)icosahydro-1H-cyclopenta[a]chrysen-9-yl)oxy)-2,2-dimethyl-4-oxobutanoic acid,
4-(((1R,3aS,5aR,5bR,7aR,9S,11aR,11bR,13aR,13bR)-3a-(((15R,16R)-16-(1,1-dioxidothiomorpholino)-15-methyl-13-oxo-2,5,8,11,14-pentaoxaheptadecan-17-yl)amino)-5a,5b,8,8,11a-pentamethyl-1-(1-methylcyclopropyl)icosahydro-1H-cyclopenta[a]chrysen-9-yl)oxy)-2,2-dimethyl-4-oxobutanoic acid,
4-(((1S,3aS,5aR,5bR,7aR,9S,11aR,11bR,13aR,13bR)-3a-(((15R,16R)-16-(1,1-dioxidothiomorpholino)-15-methyl-13-oxo-2,5,8,11,14-pentaoxaheptadecan-17-yl)amino)-1-isopropyl-5a,5b,8,8,11a-pentamethylicosahydro-1H-cyclopenta[a]chrysen-9-yl)oxy)-2,2-dimethyl-4-oxobutanoic acid,
5-(((1R,3aS,5aR,5bR,7aR,9S,11aR,11bR,13aR,13bR)-3a-(((15R,16R)-16-(1,1-dioxidothiomorpholino)-15-methyl-13-oxo-2,5,8,11,14-pentaoxaheptadecan-17-yl)amino)-5a,5b,8,8,11a-pentamethyl-1-(prop-1-en-2-yl)icosahydro-1H-cyclopenta[a]chrysen-9-yl)oxy)-3,3-dimethyl-5-oxopentanoic acid,
5-(((1R,3aS,5aR,5bR,7aR,9S,11aR,11bR,13aR,13bR)-3a-(((15R,16R)-16-(1,1-dioxidothiomorpholino)-15-methyl-13-oxo-2,5,8,11,14-pentaoxaheptadecan-17-yl)amino)-5a,5b,8,8,11a-pentamethyl-1-(1-methylcyclopropyl)icosahydro-1H-cyclopenta[a]chrysen-9-yl)oxy)-3,3-dimethyl-5-oxopentanoic acid,
5-(((1S,3aS,5aR,5bR,7aR,9S,11aR,11bR,13aR,13bR)-3a-(((15R,16R)-16-(1,1-dioxidothiomorpholino)-15-methyl-13-oxo-2,5,8,11,14-pentaoxaheptadecan-17-yl)amino)-1-isopropyl-5a,5b,8,8,11a-pentamethylicosahydro-1H-cyclopenta[a]chrysen-9-yl)oxy)-3,3-dimethyl-5-oxopentanoic acid,
(1R,3S)-3-((((1R,3aS,5aR,5bR,7aR,9S,11aR,11bR,13aR,13bR)-3a-(((S)-3-amino-2-(1,1-dioxidothiomorpholino)propyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((S)-3-amino-2-(1,1-dioxidothiomorpholino)propyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1R,3S)-3-((((1S,3aS,5aR,5bR,7aR,9S,11aR,11bR,13aR,13bR)-3a-(((S)-3-amino-2-(1,1-dioxidothiomorpholino)propyl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((2R,3R)-3-amino-2-(1,1-dioxidothiomorpholino)butyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((2R,3R)-3-amino-2-(1,1-dioxidothiomorpholino)butyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1R,3S)-3-((((1S,3aS,5aR,5bR,7aR,9S,11aR,11bR,13aR,13bR)-3a-(((2R,3R)-3-amino-2-(1,1-dioxidothiomorpholino)butyl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((R)-3-((tert-butoxycarbonyl)amino)-2-(1,1-dioxidothiomorpholino)propyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((R)-3-((tert-butoxycarbonyl)amino)-2-(1,1-dioxidothiomorpholino)propyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1R,3S)-3-((((1S,3aS,5aR,5bR,7aR,9S,11aR,11bR,13aR,13bR)-3a-(((R)-3-((tert-butoxycarbonyl)amino)-2-(1,1-dioxidothiomorpholino)propyl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((2R,3R)-3-((tert-butoxycarbonyl)amino)-2-(1,1-dioxidothiomorpholino)butyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((2R,3R)-3-((tert-butoxycarbonyl)amino)-2-(1,1-dioxidothiomorpholino)butyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1R,3S)-3-((((1S,3aS,5aR,5bR,7aR,9S,11aR,11bR,13aR,13bR)-3a-(((2R,3R)-3-((tert-butoxycarbonyl)amino)-2-(1,1-dioxidothiomorpholino)butyl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((R)-2-(1,1-dioxidothiomorpholino)-3-((ethoxycarbonyl)amino)propyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((R)-2-(1,1-dioxidothiomorpholino)-3-((ethoxycarbonyl)amino)propyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1R,3S)-3-((((1S,3aS,5aR,5bR,7aR,9S,11aR,11bR,13aR,13bR)-3a-(((R)-2-(1,1-dioxidothiomorpholino)-3-((ethoxycarbonyl)amino)propyl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((2R,3R)-2-(1,1-dioxidothiomorpholino)-3-((ethoxycarbonyl)amino)butyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((2R,3R)-2-(1,1-dioxidothiomorpholino)-3-((ethoxycarbonyl)amino)butyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1R,3S)-3-((((1S,3aS,5aR,5bR,7aR,9S,11aR,11bR,13aR,13bR)-3a-(((2R,3R)-2-(1,1-dioxidothiomorpholino)-3-((ethoxycarbonyl)amino)butyl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((R)-2-(1,1-dioxidothiomorpholino)-3-((isopropoxycarbonyl)amino)propyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((R)-2-(1,1-dioxidothiomorpholino)-3-((isopropoxycarbonyl)amino)propyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1R,3S)-3-((((1S,3aS,5aR,5bR,7aR,9S,11aR,11bR,13aR,13bR)-3a-(((R)-2-(1,1-dioxidothiomorpholino)-3-((isopropoxycarbonyl)amino)propyl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((2R,3R)-2-(1,1-dioxidothiomorpholino)-3-((isopropoxycarbonyl)amino)butyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((2R,3R)-2-(1,1-dioxidothiomorpholino)-3-((isopropoxycarbonyl)amino)butyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1R,3S)-3-((((1S,3aS,5aR,5bR,7aR,9S,11aR,11bR,13aR,13bR)-3a-(((2R,3R)-2-(1,1-dioxidothiomorpholino)-3-((isopropoxycarbonyl)amino)butyl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((R)-2-(1,1-dioxidothiomorpholino)-3-(((2-methoxyethoxy)carbonyl)amino)propyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((R)-2-(1,1-dioxidothiomorpholino)-3-(((2-methoxyethoxy)carbonyl)amino)propyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1R,3S)-3-((((1S,3aS,5aR,5bR,7aR,9S,11aR,11bR,13aR,13bR)-3a-(((R)-2-(1,1-dioxidothiomorpholino)-3-(((2-methoxyethoxy)carbonyl)amino)propyl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((2R,3R)-2-(1,1-dioxidothiomorpholino)-3-(((2-methoxyethoxy)carbonyl)amino)butyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((2R,3R)-2-(1,1-dioxidothiomorpholino)-3-(((2-methoxyethoxy)carbonyl)amino)butyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1R,3S)-3-((((1S,3aS,5aR,5bR,7aR,9S,11aR,11bR,13aR,13bR)-3a-(((2R,3R)-2-(1,1-dioxidothiomorpholino)-3-(((2-methoxyethoxy)carbonyl)amino)butyl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((R)-12-(1,1-dioxidothiomorpholino)-9-oxo-2,5,8-trioxa-10-azatridecan-13-yl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((R)-12-(1,1-dioxidothiomorpholino)-9-oxo-2,5,8-trioxa-10-azatridecan-13-yl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1R,3S)-3-((((1S,3aS,5aR,5bR,7aR,9S,11aR,11bR,13aR,13bR)-3a-(((R)-12-(1,1-dioxidothiomorpholino)-9-oxo-2,5,8-trioxa-10-azatridecan-13-yl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((11R,12R)-12-(1,1-dioxidothiomorpholino)-11-methyl-9-oxo-2,5,8-trioxa-10-azatridecan-13-yl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((11R,12R)-12-(1,1-dioxidothiomorpholino)-11-methyl-9-oxo-2,5,8-trioxa-10-azatridecan-13-yl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1R,3S)-3-((((1S,3aS,5aR,5bR,7aR,9S,11aR,11bR,13aR,13bR)-3a-(((11R,12R)-12-(1,1-dioxidothiomorpholino)-11-methyl-9-oxo-2,5,8-trioxa-10-azatridecan-13-yl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((R)-2-(1,1-dioxidothiomorpholino)-3-((1-methylethyl)sulfonamido)propyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((R)-2-(1,1-dioxidothiomorpholino)-3-((1-methylethyl)sulfonamido)propyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1R,3S)-3-((((1S,3aS,5aR,5bR,7aR,9S,11aR,11bR,13aR,13bR)-3a-(((R)-2-(1,1-dioxidothiomorpholino)-3-((1-methylethyl)sulfonamido)propyl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((2R,3R)-2-(1,1-dioxidothiomorpholino)-3-((1-methylethyl)sulfonamido)butyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((2R,3R)-2-(1,1-dioxidothiomorpholino)-3-((1-methylethyl)sulfonamido)butyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1R,3S)-3-((((1S,3aS,5aR,5bR,7aR,9S,11aR,11bR,13aR,13bR)-3a-(((2R,3R)-2-(1,1-dioxidothiomorpholino)-3-((1-methylethyl)sulfonamido)butyl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((R)-2-(1,1-dioxidothiomorpholino)-3-(methylthio)propyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((R)-2-(1,1-dioxidothiomorpholino)-3-(methylthio)propyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((S)-2-(1,1-dioxidothiomorpholino)-4-(methylthio)butyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((S)-2-(1,1-dioxidothiomorpholino)-4-(methylthio)butyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
4-(((1R,3aS,5aR,5bR,7aR,9S,11aR,11bR,13aR,13bR)-3a-(((S)-3-amino-2-(1,1-dioxidothiomorpholino)propyl)amino)-5a,5b,8,8,11a-pentamethyl-1-(prop-1-en-2-yl)icosahydro-1H-cyclopenta[a]chrysen-9-yl)oxy)-2,2-dimethyl-4-oxobutanoic acid,
5-(((1R,3aS,5aR,5bR,7aR,9S,11aR,11bR,13aR,13bR)-3a-(((S)-3-amino-2-(1,1-dioxidothiomorpholino)propyl)amino)-5a,5b,8,8,11a-pentamethyl-1-(prop-1-en-2-yl)icosahydro-1H-cyclopenta[a]chrysen-9-yl)oxy)-3,3-dimethyl-5-oxopentanoic acid,
4-(((1R,3aS,5aR,5bR,7aR,9S,11aR,11bR,13aR,13bR)-3a-(((2R,3R)-3-amino-2-(1,1-dioxidothiomorpholino)butyl)amino)-5a,5b,8,8,11a-pentamethyl-1-(prop-1-en-2-yl)icosahydro-1H-cyclopenta[a]chrysen-9-yl)oxy)-2,2-dimethyl-4-oxobutanoic acid,
5-(((1R,3aS,5aR,5bR,7aR,9S,11aR,11bR,13aR,13bR)-3a-(((2R,3R)-3-amino-2-(1,1-dioxidothiomorpholino)butyl)amino)-5a,5b,8,8,11a-pentamethyl-1-(prop-1-en-2-yl)icosahydro-1H-cyclopenta[a]chrysen-9-yl)oxy)-3,3-dimethyl-5-oxopentanoic acid,
4-(((1R,3aS,5aR,5bR,7aR,9S,11aR,11bR,13aR,13bR)-3a-(((R)-2-(1,1-dioxidothiomorpholino)-3-((ethoxycarbonyl)amino)propyl)amino)-5a,5b,8,8,11a-pentamethyl-1-(prop-1-en-2-yl)icosahydro-1H-cyclopenta[a]chrysen-9-yl)oxy)-2,2-dimethyl-4-oxobutanoic acid,
5-(((1R,3aS,5aR,5bR,7aR,9S,11aR,11bR,13aR,13bR)-3a-(((R)-2-(1,1-dioxidothiomorpholino)-3-((ethoxycarbonyl)amino)propyl)amino)-5a,5b,8,8,11a-pentamethyl-1-(prop-1-en-2-yl)icosahydro-1H-cyclopenta[a]chrysen-9-yl)oxy)-3,3-dimethyl-5-oxopentanoic acid,
4-(((1R,3aS,5aR,5bR,7aR,9S,11aR,11bR,13aR,13bR)-3a-(((2R,3R)-2-(1,1-dioxidothiomorpholino)-3-((ethoxycarbonyl)amino)butyl)amino)-5a,5b,8,8,11a-pentamethyl-1-(prop-1-en-2-yl)icosahydro-1H-cyclopenta[a]chrysen-9-yl)oxy)-2,2-dimethyl-4-oxobutanoic acid,
5-(((1R,3aS,5aR,5bR,7aR,9S,11aR,11bR,13aR,13bR)-3a-(((2R,3R)-2-(1,1-dioxidothiomorpholino)-3-((ethoxycarbonyl)amino)butyl)amino)-5a,5b,8,8,11a-pentamethyl-1-(prop-1-en-2-yl)icosahydro-1H-cyclopenta[a]chrysen-9-yl)oxy)-3,3-dimethyl-5-oxopentanoic acid,
4-(((1R,3aS,5aR,5bR,7aR,9S,11aR,11bR,13aR,13bR)-3a-(((R)-2-(1,1-dioxidothiomorpholino)-3-((1-methylethyl)sulfonamido)propyl)amino)-5a,5b,8,8,11a-pentamethyl-1-(prop-1-en-2-yl)icosahydro-1H-cyclopenta[a]chrysen-9-yl)oxy)-2,2-dimethyl-4-oxobutanoic acid,
5-(((1R,3aS,5aR,5bR,7aR,9S,11aR,11bR,13aR,13bR)-3a-(((R)-2-(1,1-dioxidothiomorpholino)-3-((1-methylethyl)sulfonamido)propyl)amino)-5a,5b,8,8,11a-pentamethyl-1-(prop-1-en-2-yl)icosahydro-1H-cyclopenta[a]chrysen-9-yl)oxy)-3,3-dimethyl-5-oxopentanoic acid,
4-(((1R,3aS,5aR,5bR,7aR,9S,11aR,11bR,13aR,13bR)-3a-(((2R,3R)-2-(1,1-dioxidothiomorpholino)-3-((1-methylethyl)sulfonamido)butyl)amino)-5a,5b,8,8,11a-pentamethyl-1-(prop-1-en-2-yl)icosahydro-1H-cyclopenta[a]chrysen-9-yl)oxy)-2,2-dimethyl-4-oxobutanoic acid,
5-(((1R,3aS,5aR,5bR,7aR,9S,11aR,11bR,13aR,13bR)-3a-(((2R,3R)-2-(1,1-dioxidothiomorpholino)-3-((1-methylethyl)sulfonamido)butyl)amino)-5a,5b,8,8,11a-pentamethyl-1-(prop-1-en-2-yl)icosahydro-1H-cyclopenta[a]chrysen-9-yl)oxy)-3,3-dimethyl-5-oxopentanoic acid,
4-(((1R,3aS,5aR,5bR,7aR,9S,11aR,11bR,13aR,13bR)-3a-(((R)-2-(1,1-dioxidothiomorpholino)-3-(methylthio)propyl)amino)-5a,5b,8,8,11a-pentamethyl-1-(prop-1-en-2-yl)icosahydro-1H-cyclopenta[a]chrysen-9-yl)oxy)-2,2-dimethyl-4-oxobutanoic acid,
5-(((1R,3aS,5aR,5bR,7aR,9S,11aR,11bR,13aR,13bR)-3a-(((R)-2-(1,1-dioxidothiomorpholino)-3-(methylthio)propyl)amino)-5a,5b,8,8,11a-pentamethyl-1-(prop-1-en-2-yl)icosahydro-1H-cyclopenta[a]chrysen-9-yl)oxy)-3,3-dimethyl-5-oxopentanoic acid,
4-(((1R,3aS,5aR,5bR,7aR,9S,11aR,11bR,13aR,13bR)-3a-(((S)-2-(1,1-dioxidothiomorpholino)-4-(methylthio)butyl)amino)-5a,5b,8,8,11a-pentamethyl-1-(prop-1-en-2-yl)icosahydro-1H-cyclopenta[a]chrysen-9-yl)oxy)-2,2-dimethyl-4-oxobutanoic acid,
5-(((1R,3aS,5aR,5bR,7aR,9S,11aR,11bR,13aR,13bR)-3a-(((S)-2-(1,1-dioxidothiomorpholino)-4-(methylthio)butyl)amino)-5a,5b,8,8,11a-pentamethyl-1-(prop-1-en-2-yl)icosahydro-1H-cyclopenta[a]chrysen-9-yl)oxy)-3,3-dimethyl-5-oxopentanoic acid,
4-(((1R,3aS,5aR,5bR,7aR,9S,11aR,11bR,13aR,13bR)-3a-(((S)-2-(1,1-dioxidothiomorpholino)-3-(methylthio)propyl)amino)-5a,5b,8,8,11a-pentamethyl-1-(prop-1-en-2-yl)icosahydro-1H-cyclopenta[a]chrysen-9-yl)oxy)-2,2-dimethyl-4-oxobutanoic acid,
4-(((1R,3aS,5aR,5bR,7aR,9S,11aR,11bR,13aR,13bR)-3a-(((S)-2-(1,1-dioxidothiomorpholino)-3-(methylthio)propyl)amino)-5a,5b,8,8,11a-pentamethyl-1-(1-methylcyclopropyl)icosahydro-1H-cyclopenta[a]chrysen-9-yl)oxy)-2,2-dimethyl-4-oxobutanoic acid,
4-(((1S,3aS,5aR,5bR,7aR,9S,11aR,11bR,13aR,13bR)-3a-(((S)-2-(1,1-dioxidothiomorpholino)-3-(methylthio)propyl)amino)-1-isopropyl-5a,5b,8,8,11a-pentamethylicosahydro-1H-cyclopenta[a]chrysen-9-yl)oxy)-2,2-dimethyl-4-oxobutanoic acid,
4-(((1R,3aS,5aR,5bR,7aR,9S,11aR,11bR,13aR,13bR)-3a-(((R)-2-(1,1-dioxidothiomorpholino)-4-(methylthio)butyl)amino)-5a,5b,8,8,11a-pentamethyl-1-(prop-1-en-2-yl)icosahydro-1H-cyclopenta[a]chrysen-9-yl)oxy)-2,2-dimethyl-4-oxobutanoic acid,
4-(((1R,3aS,5aR,5bR,7aR,9S,11aR,11bR,13aR,13bR)-3a-(((R)-2-(1,1-dioxidothiomorpholino)-4-(methylthio)butyl)amino)-5a,5b,8,8,11a-pentamethyl-1-(1-methylcyclopropyl)icosahydro-1H-cyclopenta[a]chrysen-9-yl)oxy)-2,2-dimethyl-4-oxobutanoic acid,
4-(((1S,3aS,5aR,5bR,7aR,9S,11aR,11bR,13aR,13bR)-3a-(((R)-2-(1,1-dioxidothiomorpholino)-4-(methylthio)butyl)amino)-1-isopropyl-5a,5b,8,8,11a-pentamethylicosahydro-1H-cyclopenta[a]chrysen-9-yl)oxy)-2,2-dimethyl-4-oxobutanoic acid,
5-(((1R,3aS,5aR,5bR,7aR,9S,11aR,11bR,13aR,13bR)-3a-(((R)-2-(1,1-dioxidothiomorpholino)-4-(methylthio)butyl)amino)-5a,5b,8,8,11a-pentamethyl-1-(prop-1-en-2-yl)icosahydro-1H-cyclopenta[a]chrysen-9-yl)oxy)-3,3-dimethyl-5-oxopentanoic acid,
5-(((1R,3aS,5aR,5bR,7aR,9S,11aR,11bR,13aR,13bR)-3a-(((R)-2-(1,1-dioxidothiomorpholino)-4-(methylthio)butyl)amino)-5a,5b,8,8,11a-pentamethyl-1-(1-methylcyclopropyl)icosahydro-1H-cyclopenta[a]chrysen-9-yl)oxy)-3,3-dimethyl-5-oxopentanoic acid,
5-(((1S,3aS,5aR,5bR,7aR,9S,11aR,11bR,13aR,13bR)-3a-(((R)-2-(1,1-dioxidothiomorpholino)-4-(methylthio)butyl)amino)-1-isopropyl-5a,5b,8,8,11a-pentamethylicosahydro-1H-cyclopenta[a]chrysen-9-yl)oxy)-3,3-dimethyl-5-oxopentanoic acid,
(1R,3S)-3-((((1R,3aS,5aR,5bR,7aR,9S,11aR,11bR,13aR,13bR)-3a-(((R)-2-(1,1-dioxidothiomorpholino)-4-(methylthio)butyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1S,3R)-3-((((1R,3aS,5aR,5bR,7aR,9S,11aR,11bR,13aR,13bR)-3a-(((R)-2-(1,1-dioxidothiomorpholino)-4-(methylthio)butyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
2-((1R,3S)-3-((((1R,3aS,5aR,5bR,7aR,9S,11aR,11bR,13aR,13bR)-3a-(((R)-2-(1,1-dioxidothiomorpholino)-4-(methylthio)butyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclopropyl)acetic acid,
2-((1R,3S)-3-((((1R,3aS,5aR,5bR,7aR,9S,11aR,11bR,13aR,13bR)-3a-(((S)-2-(1,1-dioxidothiomorpholino)-4-(methylthio)butyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclopropyl)acetic acid,
(1R,3S)-3-((((1R,3aS,5aR,5bR,7aR,9S,11aR,11bR,13aR,13bR)-3a-(((R)-2-(1,1-dioxidothiomorpholino)-3-hydroxypropyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclopropane-1-carboxylic acid,
(1R,3S)-3-((((1R,3aS,5aR,5bR,7aR,9S,11aR,11bR,13aR,13bR)-3a-(((S)-2-(1,1-dioxidothiomorpholino)-3-hydroxypropyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclopropane-1-carboxylic acid,
(1R,3S)-3-((((1R,3aS,5aR,5bR,7aR,9S,11aR,11bR,13aR,13bR)-3a-(((2R,3S)-2-(1,1-dioxidothiomorpholino)-3-fluorobutyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclopropane-1-carboxylic acid,
(1S,3R)-3-((((1R,3aS,5aR,5bR,7aR,9S,11aR,11bR,13aR,13bR)-3a-(((2R,3S)-2-(1,1-dioxidothiomorpholino)-3-fluorobutyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclopropane-1-carboxylic acid,
(1R,3S)-3-((((1R,3aS,5aR,5bR,7aR,9S,11aR,11bR,13aR,13bR)-3a-(((R)-2-(1,1-dioxidothiomorpholino)-3-fluoropropyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclopropane-1-carboxylic acid,
(1R,3S)-3-((((1R,3aS,5aR,5bR,7aR,9S,11aR,11bR,13aR,13bR)-3a-(((S)-2-(1,1-dioxidothiomorpholino)-3-fluoropropyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclopropane-1-carboxylic acid,
(1R,3S)-3-((((1R,3aS,5aR,5bR,7aR,9S,11aR,11bR,13aR,13bR)-3a-(((S)-3-amino-2-(1,1-dioxidothiomorpholino)propyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclopropane-1-carboxylic acid,
(1R,3S)-3-((((1R,3aS,5aR,5bR,7aR,9S,11aR,11bR,13aR,13bR)-3a-(((R)-3-amino-2-(1,1-dioxidothiomorpholino)propyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclopropane-1-carboxylic acid,
(1R,3S)-3-((((1R,3aS,5aR,5bR,7aR,9S,11aR,11bR,13aR,13bR)-3a-(((2R,3R)-3-amino-2-(1,1-dioxidothiomorpholino)butyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclopropane-1-carboxylic acid,
(1R,3S)-3-((((1R,3aS,5aR,5bR,7aR,9S,11aR,11bR,13aR,13bR)-3a-(((2S,3S)-3-amino-2-(1,1-dioxidothiomorpholino)butyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclopropane-1-carboxylic acid,
(1S,3R)-3-((((1R,3aS,5aR,5bR,7aR,9S,11aR,11bR,13aR,13bR)-3a-(((2R,3R)-3-amino-2-(1,1-dioxidothiomorpholino)butyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclopropane-1-carboxylic acid,
(1S,3R)-3-((((1R,3aS,5aR,5bR,7aR,9S,11aR,11bR,13aR,13bR)-3a-(((2S,3S)-3-amino-2-(1,1-dioxidothiomorpholino)butyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclopropane-1-carboxylic acid,
(1R,3S)-3-((((1R,3aS,5aR,5bR,7aR,9S,11aR,11bR,13aR,13bR)-3a-(((R)-2-(1,1-dioxidothiomorpholino)-3-(methylthio)propyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclopropane-1-carboxylic acid,
(1R,3S)-3-((((1R,3aS,5aR,5bR,7aR,9S,11aR,11bR,13aR,13bR)-3a-(((S)-2-(1,1-dioxidothiomorpholino)-3-(methylthio)propyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclopropane-1-carboxylic acid,
(1R,3S)-3-((((1R,3aS,5aR,5bR,7aR,9S,11aR,11bR,13aR,13bR)-3a-(((S)-2-(1,1-dioxidothiomorpholino)-4-(methylthio)butyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclopropane-1-carboxylic acid,
(1R,3S)-3-((((1R,3aS,5aR,5bR,7aR,9S,11aR,11bR,13aR,13bR)-3a-(((R)-2-(1,1-dioxidothiomorpholino)-4-(methylthio)butyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclopropane-1-carboxylic acid,
(1R,3S)-3-((((1R,3aS,5aR,5bR,7aR,9S,11aR,11bR,13aR,13bR)-3a-(((R)-2-(1,1-dioxidothiomorpholino)-3-methylbutyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclopropane-1-carboxylic acid,
(1R,3S)-3-((((1R,3aS,5aR,5bR,7aR,9S,11aR,11bR,13aR,13bR)-3a-(((S)-2-(1,1-dioxidothiomorpholino)-3-methylbutyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclopropane-1-carboxylic acid,
5-(((1R,3aS,5aR,5bR,7aR,9S,11aR,11bR,13aR,13bR)-3a-((3-(1,1-dioxidothiomorpholino)-4-hydroxybutyl)amino)-5a,5b,8,8,11a-pentamethyl-1-(prop-1-en-2-yl)icosahydro-1H-cyclopenta[a]chrysen-9-yl)oxy)-3,3-dimethyl-5-oxopentanoic acid,
(1R,3S)-3-((((1R,3aS,5aR,5bR,7aR,9S,11aR,11bR,13aR,13bR)-3a-(((3S,4R)-2-(1,1-dioxidothiomorpholino)-3-hydroxybutyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((12S,13S)-13-(1,1-dioxidothiomorpholino)-12-methyl-10-oxo-2,5,8,11-tetraoxapentadecan-15-yl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((3R,4S)-3-(1,1-dioxidothiomorpholino)-4-hydroxypentyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1S,3aS,5aR,5bR,7aR,9S,11aR,11bR,13aR,13bR)-3a-(((3S,4S)-3-(1,1-dioxidothiomorpholino)-4-hydroxypentyl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1R,3S)-3-((((1S,3aS,5aR,5bR,7aR,9S,11aR,11bR,13aR,13bR)-3a-(((3R,4S)-3-(1,1-dioxidothiomorpholino)-4-hydroxypentyl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((3S,4S)-3-(1,1-dioxidothiomorpholino)-4-hydroxypentyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((3R,4S)-3-(1,1-dioxidothiomorpholino)-4-hydroxypentyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1S,3R)-3-((((1R,3aS,5aR,5bR,7aR,9S,11aR,11bR,13aR,13bR)-3a-(((3R,4R)-3-(1,1-dioxidothiomorpholino)-4-hydroxypentyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1S,3R)-3-((((1S,3aS,5aR,5bR,7aR,9S,11aR,11bR,13aR,13bR)-3a-(((3R,4R)-3-(1,1-dioxidothiomorpholino)-4-hydroxypentyl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1S,3R)-3-((((1R,3aS,5aR,5bR,7aR,9S,11aR,11bR,13aR,13bR)-3a-(((3R,4R)-3-(1,1-dioxidothiomorpholino)-4-hydroxypentyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((3R,4R)-3-(1,1-dioxidothiomorpholino)-4-hydroxypentyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclopropane-1-carboxylic acid,
(1R,3S)-3-((((1S,3aS,5aR,5bR,7aR,9S,11aR,11bR,13aR,13bR)-3a-(((3R,4R)-3-(1,1-dioxidothiomorpholino)-4-hydroxypentyl)amino)-1-isopropyl-5a,5b,8,8,11a-pentamethylicosahydro-1H-cyclopenta[a]chrysen-9-yl)oxy)carbonyl)-2,2-dimethylcyclopropane-1-carboxylic acid,
(1R,3S)-3-((((1R,3aS,5aR,5bR,7aR,9S,11aR,11bR,13aR,13bR)-3a-(((3R,4R)-3-(1,1-dioxidothiomorpholino)-4-hydroxypentyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclopropane-1-carboxylic acid,
(1R,3S)-3-((((1R,3aS,5aR,5bR,7aR,9S,11aR,11bR,13aR,13bR)-3a-(((R)-3-(1,1-dioxidothiomorpholino)-4-hydroxybutyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclopropane-1-carboxylic acid,
(1R,3S)-3-((((1R,3aS,5aR,5bR,7aR,9S,11aR,11bR,13aR,13bR)-3a-(((R)-4-amino-3-(1,1-dioxidothiomorpholino)butyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclopropane-1-carboxylic acid,
(1R,3S)-3-((((1S,3aS,5aR,5bR,7aR,9S,11aR,11bR,13aR,13bR)-3a-(((R)-3-(1,1-dioxidothiomorpholino)-4-hydroxybutyl)amino)-1-isopropyl-5a,5b,8,8,11a-pentamethylicosahydro-1H-cyclopenta[a]chrysen-9-yl)oxy)carbonyl)-2,2-dimethylcyclopropane-1-carboxylic acid,
(1R,3S)-3-((((1S,3aS,5aR,5bR,7aR,9S,11aR,11bR,13aR,13bR)-3a-(((R)-4-amino-3-(1,1-dioxidothiomorpholino)butyl)amino)-1-isopropyl-5a,5b,8,8,11a-pentamethylicosahydro-1H-cyclopenta[a]chrysen-9-yl)oxy)carbonyl)-2,2-dimethylcyclopropane-1-carboxylic acid,
(1R,3S)-3-((((1R,3aS,5aR,5bR,7aR,9S,11aR,11bR,13aR,13bR)-3a-(((R)-3-(1,1-dioxidothiomorpholino)-4-hydroxybutyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclopropane-1-carboxylic acid,
(1R,3S)-3-((((1R,3aS,5aR,5bR,7aR,9S,11aR,11bR,13aR,13bR)-3a-(((R)-4-amino-3-(1,1-dioxidothiomorpholino)butyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclopropane-1-carboxylic acid,
(1R,3S)-3-((((1R,3aS,5aR,5bR,7aR,9S,11aR,11bR,13aR,13bR)-3a-(((R)-3-(1,1-dioxidothiomorpholino)-4-fluorobutyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclopropane-1-carboxylic acid,
(1R,3S)-3-((((1S,3aS,5aR,5bR,7aR,9S,11aR,11bR,13aR,13bR)-3a-(((R)-3-(1,1-dioxidothiomorpholino)-4-fluorobutyl)amino)-1-isopropyl-5a,5b,8,8,11a-pentamethylicosahydro-1H-cyclopenta[a]chrysen-9-yl)oxy)carbonyl)-2,2-dimethylcyclopropane-1-carboxylic acid,
(1R,3S)-3-((((1R,3aS,5aR,5bR,7aR,9S,11aR,11bR,13aR,13bR)-3a-(((R)-3-(1,1-dioxidothiomorpholino)-4-fluorobutyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclopropane-1-carboxylic acid,
(1S,3R)-3-(2-(((1R,3aS,5aR,5bR,7aR,9S,11aR,11bR,13aR,13bR)-3a-(((3R,4R)-3-(1,1-dioxidothiomorpholino)-4-hydroxypentyl)amino)-5a,5b,8,8,11a-pentamethyl-1-(prop-1-en-2-yl)icosahydro-1H-cyclopenta[a]chrysen-9-yl)oxy)-2-oxoethyl)-2,2-dimethylcyclopropane-1-carboxylic acid,
2-((1R,3S)-3-((((1R,3aS,5aR,5bR,7aR,9S,11aR,11bR,13aR,13bR)-3a-(((3R,4R)-3-(1,1-dioxidothiomorpholino)-4-hydroxypentyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclopropyl)acetic acid,
4-(((1R,3aS,5aR,5bR,7aR,9S,11aR,11bR,13aR,13bR)-3a-(((3R,4R)-3-(1,1-dioxidothiomorpholino)-4-hydroxypentyl)amino)-5a,5b,8,8,11a-pentamethyl-1-(prop-1-en-2-yl)icosahydro-1H-cyclopenta[a]chrysen-9-yl)oxy)-2,2-dimethyl-4-oxobutanoic acid,
5-(((1R,3aS,5aR,5bR,7aR,9S,11aR,11bR,13aR,13bR)-3a-(((3R,4R)-3-(1,1-dioxidothiomorpholino)-4-hydroxypentyl)amino)-5a,5b,8,8,11a-pentamethyl-1-(1-1-(prop-1-en-2-yl)icosahydro-1H-cyclopenta[a]chrysen-9-yl)oxy)-2,2-dimethyl-4-oxobutanoic acid,
4-(((1R,3aS,5aR,5bR,7aR,9S,11aR,11bR,13aR,13bR)-3a-(((3S,4R)-2-(1,1-dioxidothiomorpholino)-3-hydroxybutyl)amino)-5a,5b,8,8,11a-pentamethyl-1-(prop-1-en-2-yl)icosahydro-1H-cyclopenta[a]chrysen-9-yl)oxy)-2,2-dimethyl-4-oxobutanoic acid,
4-(((1S,3aS,5aR,5bR,7aR,9S,11aR,11bR,13aR,13bR)-3a-(((3R,4R)-3-(1,1-dioxidothiomorpholino)-4-hydroxypentyl)amino)-1-isopropyl-5a,5b,8,8,11a-pentamethylicosahydro-1H-cyclopenta[a]chrysen-9-yl)oxy)-2,2-dimethyl-4-oxobutanoic acid,
4-(((1S,3aS,5aR,5bR,7aR,9S,11aR,11bR,13aR,13bR)-3a-(((3R,4S)-3-(1,1-dioxidothiomorpholino)-4-hydroxypentyl)amino)-1-isopropyl-5a,5b,8,8,11a-pentamethylicosahydro-1H-cyclopenta[a]chrysen-9-yl)oxy)-2,2-dimethyl-4-oxobutanoic acid,
4-(((1R,3aS,5aR,5bR,7aR,9S,11aR,11bR,13aR,13bR)-3a-(((3R,4R)-3-(1,1-dioxidothiomorpholino)-4-hydroxypentyl)amino)-5a,5b,8,8,11a-pentamethyl-1-(1-methylcyclopropyl)icosahydro-1H-cyclopenta[a]chrysen-9-yl)oxy)-2,2-dimethyl-4-oxobutanoic acid,
4-(((1R,3aS,5aR,5bR,7aR,9S,11aR,11bR,13aR,13bR)-3a-(((3R,4S)-3-(1,1-dioxidothiomorpholino)-4-hydroxypentyl)amino)-5a,5b,8,8,11a-pentamethyl-1-(1-methylcyclopropyl)icosahydro-1H-cyclopenta[a]chrysen-9-yl)oxy)-2,2-dimethyl-4-oxobutanoic acid,
5-(((1R,3aS,5aR,5bR,7aR,9S,11aR,11bR,13aR,13bR)-3a-(((3R,4R)-3-(1,1-dioxidothiomorpholino)-4-hydroxypentyl)amino)-5a,5b,8,8,11a-pentamethyl-1-(prop-1-en-2-yl)icosahydro-1H-cyclopenta[a]chrysen-9-yl)oxy)-3,3-dimethyl-5-oxopentanoic acid,
5-(((1R,3aS,5aR,5bR,7aR,9S,11aR,11bR,13aR,13bR)-3a-(((3R,4S)-3-(1,1-dioxidothiomorpholino)-4-hydroxypentyl)amino)-5a,5b,8,8,11a-pentamethyl-1-(prop-1-en-2-yl)icosahydro-1H-cyclopenta[a]chrysen-9-yl)oxy)-3,3-dimethyl-5-oxopentanoic acid,
5-(((1S,3aS,5aR,5bR,7aR,9S,11aR,11bR,13aR,13bR)-3a-(((3R,4R)-3-(1,1-dioxidothiomorpholino)-4-hydroxypentyl)amino)-1-isopropyl-5a,5b,8,8,11a-pentamethylicosahydro-1H-cyclopenta[a]chrysen-9-yl)oxy)-3,3-dimethyl-5-oxopentanoic acid,
5-(((1S,3aS,5aR,5bR,7aR,9S,11aR,11bR,13aR,13bR)-3a-(((3R,4S)-3-(1,1-dioxidothiomorpholino)-4-hydroxypentyl)amino)-1-isopropyl-5a,5b,8,8,11a-pentamethylicosahydro-1H-cyclopenta[a]chrysen-9-yl)oxy)-3,3-dimethyl-5-oxopentanoic acid,
(1R,3S)-3-((((1R,3aS,5aR,5bR,7aR,9S,11aR,11bR,13aR,13bR)-3a-(((R)-3-(1,1-methylcyclopropyl)icosahydro-1H-cyclopenta[a]chrysen-9-yl)oxy)-3,3-dimethyl-5-oxopentanoic acid,
5-(((1R,3aS,5aR,5bR,7aR,9S,11aR,11bR,13aR,13bR)-3a-(((3R,4S)-3-(1,1-dioxidothiomorpholino)-4-hydroxypentyl)amino)-5a,5b,8,8,11a-pentamethyl-1-(1-methylcyclopropyl)icosahydro-1H-cyclopenta[a]chrysen-9-yl)oxy)-3,3-dimethyl-5-oxopentanoic acid,
(1R,3S)-3-((((1R,3aS,5aR,5bR,7aR,9S,11aR,11bR,13aR,13bR)-3a-(((R)-3-(1,1-dioxidothiomorpholino)-4-hydroxybutyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((R)-4-((dimethoxyphosphoryl)oxy)-3-(1,1-dioxidothiomorpholino)butyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((R)-13-(1,1-dioxidothiomorpholino)-10-oxo-2,5,8,11-tetraoxapentadecan-15-yl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((S)-3-(1,1-dioxidothiomorpholino)-4-hydroxybutyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1S,3aS,5aR,5bR,7aR,9S,11aR,11bR,13aR,13bR)-3a-(((R)-3-(1,1-dioxidothiomorpholino)-4-hydroxybutyl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1R,3S)-3-((((1S,3aS,5aR,5bR,7aR,9S,11aR,11bR,13aR,13bR)-3a-(((S)-3-(1,1-dioxidothiomorpholino)-4-hydroxybutyl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1R,3S)-3-((((1S,3aS,5aR,5bR,7aR,9S,11aR,11bR,13aR,13bR)-3a-(((S)-3-(1,1-dioxidothiomorpholino)-4-hydroxybutyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((R)-13-(1,1-dioxidothiomorpholino)-10-oxo-2,5,8,11-tetraoxapentadecan-15-yl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((S)-3-(1,1-dioxidothiomorpholino)-4-hydroxybutyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((R)-4-amino-3-(1,1-dioxidothiomorpholino)butyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((R)-3-(1,1-dioxidothiomorpholino)-4-((ethoxycarbonyl)amino)butyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((S)-4-amino-3-(1,1-dioxidothiomorpholino)butyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1S,3aS,5aR,5bR,7aR,9S,11aR,11bR,13aR,13bR)-3a-(((R)-4-amino-3-(1,1-dioxidothiomorpholino)butyl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1R,3S)-3-((((1S,3aS,5aR,5bR,7aR,9S,11aR,11bR,13aR,13bR)-3a-(((S)-4-amino-3-(1,1-dioxidothiomorpholino)butyl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((R)-4-amino-3-(1,1-dioxidothiomorpholino)butyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((S)-4-amino-3-(1,1-dioxidothiomorpholino)butyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((R)-2-amino-3-(1,1-dioxidothiomorpholino)propyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((R)-3-(1,1-dioxidothiomorpholino)-2-((ethoxycarbonyl)amino)propyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((R)-3-(1,1-dioxidothiomorpholino)-2-(((2-methoxyethoxy)carbonyl)amino)propyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((S)-2-amino-3-(1,1-dioxidothiomorpholino)propyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1S,3aS,5aR,5bR,7aR,9S,11aR,11bR,13aR,13bR)-3a-(((R)-2-amino-3-(1,1-dioxidothiomorpholino)propyl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1R,3S)-3-((((1S,3aS,5aR,5bR,7aR,9S,11aR,11bR,13aR,13bR)-3a-(((S)-2-amino-3-(1,1-dioxidothiomorpholino)propyl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((R)-2-amino-3-(1,1-dioxidothiomorpholino)propyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((S)-2-amino-3-(1,1-dioxidothiomorpholino)propyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((R)-3-(1,1-dioxidothiomorpholino)-4-fluorobutyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((S)-3-(1,1-dioxidothiomorpholino)-4-fluorobutyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1S,3aS,5aR,5bR,7aR,9S,11aR,11bR,13aR,13bR)-3a-(((R)-3-(1,1-dioxidothiomorpholino)-4-fluorobutyl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1R,3S)-3-((((1S,3aS,5aR,5bR,7aR,9S,11aR,11bR,13aR,13bR)-3a-(((S)-3-(1,1-dioxidothiomorpholino)-4-fluorobutyl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((R)-3-(1,1-dioxidothiomorpholino)-4-fluorobutyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((S)-3-(1,1-dioxidothiomorpholino)-4-fluorobutyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((S)-3-(1,1-dioxidothiomorpholino)butyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1S,3aS,5aR,5bR,7aR,9S,11aR,11bR,13aR,13bR)-3a-(((S)-3-(1,1-dioxidothiomorpholino)butyl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((S)-3-(1,1-dioxidothiomorpholino)butyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid, and
(1R,3S)-3-((((1R,3aS,5aR,5bR,7aR,9S,11aR,11bR,13aR,13bR)-3a-(((R)-3-(1,1-dioxidothiomorpholino)butyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid, or
   pharmaceutically acceptable salts, pharmaceutically acceptable stereoisomers, prodrugs, or combination thereof.

The present invention also provides a pharmaceutical composition that includes at least one compound as described herein and at least one pharmaceutically acceptable excipient (such as a pharmaceutically acceptable carrier or diluent). Specifically, the pharmaceutical composition comprises a therapeutically effective amount of at least one compound described herein. The compound(s) present in the composition may be associated with a pharmaceutically acceptable excipient (such as a carrier or a diluent) or may be diluted by a carrier, or enclosed within a carrier which may be in the form of a capsule, sachet, or other container.

The compounds and pharmaceutical compositions described herein are useful in the treatment of diseases, conditions and/or disorders mediated by viral infections.

The present invention further provides a method of treating a disease, condition and/or disorder mediated by viral infections in a subject in need thereof by administering to the subject one or more compounds described herein in a therapeutically effective amount to cure that infection, specifically in the form of a pharmaceutical composition.

Also provided herein are processes for preparing compounds described herein.

The invention provides a method for preventing; ameliorating or treating a HIV mediated disease, disorder or syndrome in a subject in need thereof comprising administering to the subject a therapeutically effective amount of a compound of the invention. The invention further provides a method, wherein the HIV mediated disease, disorder or syndrome is like AIDS, AIDS related complex, or a syndrome characterized by symptoms such as persistent generalized lymphadenopathy, fever and weight loss, or a retroviral infection genetically related to AIDS.

Anti HIV inhibitory potential of the compounds of present invention may be demonstrated by any one or more methodologies known in the art, such as by using the assays described in Mossman T, December 1983, Journal of immunological methods, 65 (1-2), 55-63 and SPC Cole, cancer chemotherapy and Pharmacology, 1986, 17, 259-263.

### DETAILED DESCRIPTION OF THE INVENTION

The present invention provides novel triterpene derivatives and related compounds, which may be used as antiviral particularly as anti-HIV compounds and processes for the synthesis of these compounds and their pharmaceutically acceptable salts thereof, together with pharmaceutically acceptable carriers, excipients or diluents, which can be used for the treatment of diseases, condition and/or disorders mediated by viral infections, are also provided.

### The Following Definitions Apply to the Terms as Used Herein

The term “alkyl” refers to a straight or branched hydrocarbon chain radical consisting solely of carbon and hydrogen atoms, containing no unsaturation, having from one to eight carbon atoms, and which is attached to the rest of the molecule by a single bond, e.g., methyl, ethyl, n-propyl, 1-methylethyl (isopropyl), n-butyl, n-pentyl, and 1,1-dimethylethyl (t-butyl).

The term “alkenyl” refers to a hydrocarbon chain that may be a straight chain or branched chain, containing the indicated number of carbon atoms having at least one —C═C—, for example, a C₂-C₆ alkenyl group may have from 2 to 6 (inclusive) —C═C— atoms in it. Examples of C₂-C₆ alkenyl groups include, but are not limited to ethylene, prop-1-ene, but-1-ene, but-2-ene, pent-1-ene, pent-2-ene, hex-1-ene, hex-2-ene and the like.

The term “alkoxy” refers to a straight or branched hydrocarbon chain with oxygen radical consisting carbon and hydrogen atoms, containing saturation or unsaturation, having from one to eight carbon atoms, and which is attached through oxygen atom to the rest of the molecule by a single bond. One of the hydrogen atoms in the alkoxy group may be further substituted by one or more alkoxy groups to further increase the alkoxy chain length. e.g., methyloxy, ethyloxy, isopropoxy, n-propyloxy, t-butyloxy, 1-methylethyloxy (isopropyloxy), n-butyloxy, n-pentyloxy, 1,1-dimethylethyloxy (t-butyloxy),

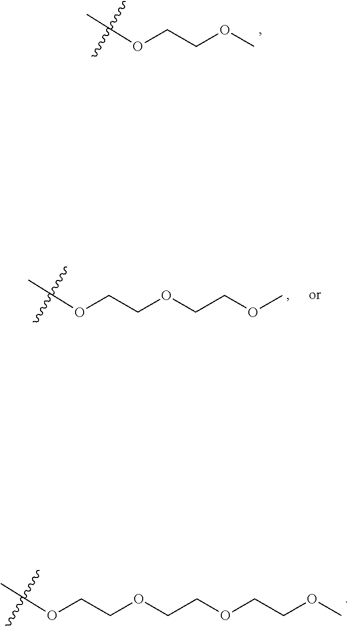

The term “cycloalkyl” denotes a non-aromatic mono or multicyclic ring system of from 3 to about 12 carbon atoms, such as but are not limited to, cyclopropyl, cyclobutyl, cyclopentyl, and cyclohexyl. Examples of multicyclic cycloalkyl groups include, but are not limited to, perhydronapththyl, adamantyl and norbornyl groups, bridged cyclic groups and spirobicyclic groups, e.g., spiro (4,4) non-2-yl.

The terms “halogen” or “halo” includes fluorine, chlorine, bromine, or iodine.

The term “hydroxy” refers to —OH.

The term “optionally substituted” refers to replacement of one or more hydrogen radicals in a given structure with a radical of a specified substituent including, but are not limited to: hydroxy, halo, carboxyl, cyano (CN), nitro, oxo (═O), thio (═S), alkyl, methyl sulfonyl, haloalkyl, alkoxy, alkenyl, aryl, aralkyl, cycloalkyl, cycloalkylalkyl, amino, —C(O)O-alkyl, heterocyclyl, heteroaryl, heterocyclylalkyl, heteroarylalkyl, alkylthio, arylthio, aryloxy, amino carbonyl, alkoxycarbonyl, alkylamino, arylamino, acyl, carboxylic acid, sulfonic acid, sulfonyl, phosphonic acid, and aliphatic. It is understood that the substituent may be further substituted.

The term “prodrug” denotes a derivative of a compound, which derivative, when administered to warm blooded animals, e.g. humans, is converted into the compound (drug). The enzymatic and/or chemical hydrolytic cleavage of the compounds of the present invention occurs in such a manner that the proven drug form (parent carboxylic acid drug) is released, and the moiety or moieties split off remain nontoxic or are metabolized so that nontoxic metabolic products are produced. For example, a carboxylic acid group can be esterified, e.g., with a methyl group or ethyl group to yield an ester. When an ester is administered to a subject, the ester is cleaved, enzymatically or non-enzymatically, reductively, oxidatively, or hydrolytically, to reveal the anionic group. An anionic group can be esterified with moieties (e.g., acyloxymethyl esters) which are cleaved to reveal an intermediate compound which subsequently decomposes to yield the active compound. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed Edward B. Roche, American Pharmaceutical Association and Pergamon Press, 1987.

The term “treating” or “treatment” of a state, disease, disorder or condition includes:
(1) preventing or delaying the appearance of clinical symptoms of the state, disease, disorder or condition developing in a subject that may be afflicted with or predisposed to the state, disease, disorder or condition but does not yet experience or display clinical or subclinical symptoms of the state, disease, disorder or condition;
(2) inhibiting the state, disease, disorder or condition, i.e., arresting or reducing the development of the state, disease, disorder or condition or at least one clinical or subclinical symptom thereof; or
(3) relieving the state, disease, disorder or condition, i.e., causing regression of the state, disease, disorder or condition or at least one of its clinical or subclinical symptoms.

The benefit to a subject receiving treatment is either statistically significant or at least perceptible to the subject or to the physician.

The term “subject” includes mammals (especially humans) and other animals, such as domestic animals (e.g., household pets including cats and dogs) and non-domestic animals (such as wildlife).

A “therapeutically effective amount” means the amount of a compound that, when administered to a subject for treating a state, disease, disorder or condition, is sufficient to effect such treatment. The “therapeutically effective amount” will vary depending on the compound, the state, disease, disorder or condition and its severity and the age, weight, physical condition and responsiveness of the subject receiving treatment.

The compounds of the present invention may form salts. Non-limiting examples of pharmaceutically acceptable salts forming part of this invention include salts derived from inorganic bases salts of organic bases salts of chiral bases, salts of natural amino acids and salts of non-natural amino acids. Certain compounds of the present invention are capable of existing in stereoisomeric forms (e.g., diastereomers, enantiomers, racemates, and combinations thereof). With respect to the overall compounds described by the formula (I), the present invention extends to these stereoisomeric forms and to mixtures thereof. To the extent prior art teaches synthesis or separation of particular stereoisomers, the different stereo isomeric forms of the present invention may be separated from one another by the methods known in the art, or a given isomer may be obtained by stereospecific or asymmetric synthesis. Tautomeric forms and mixtures of compounds described herein are also contemplated.

### Pharmaceutical Compositions

The pharmaceutical compositions provided in the present invention include at least one compound described herein and at least one pharmaceutically acceptable excipient (such as a pharmaceutically acceptable carrier or diluent). Specifically, the contemplated pharmaceutical compositions include a compound(s) described herein in an amount sufficient to treat viral infection in a subject.

The subjects contemplated include, for example, a living cell and a mammal, including human. The compound of the present invention may be associated with a pharmaceutically acceptable excipient (such as a carrier or a diluent) or be diluted by a carrier, or enclosed within a carrier which can be in the form of a capsule, sachet, or other container.

Examples of suitable carriers include, but are not limited to, water, salt solutions, alcohols, polyethylene glycols, peanut oil, olive oil, gelatin, lactose, terra alba, sucrose, dextrin, magnesium carbonate, sugar, amylose, magnesium stearate, talc, gelatin, agar, pectin, acacia, stearic acid, lower alkyl ethers of cellulose, silicic acid, fatty acids, fatty acid amines, fatty acid monoglycerides and diglycerides, fatty acid esters, and polyoxyethylene.

The carrier or diluent may include a sustained release material, such as, for example, glyceryl monostearate or glyceryl distearate, alone or mixed with a wax.

The pharmaceutical composition may also include one or more pharmaceutically acceptable auxiliary agents, wetting agents, emulsifying agents, suspending agents, preserving agents, salts for influencing osmotic pressure, buffers, sweetening agents, flavoring agents, colorants, or any combination of the foregoing. The pharmaceutical composition of the invention may be formulated so as to provide quick-, sustained-, or delayed-release of the active ingredient after administration to the subject by employing procedures known in the art.

The pharmaceutical compositions described herein may be prepared, e.g., as described in Remington: The Science and Practice of Pharmacy, 20^{th} Ed., 2003 (Lippincott Williams & Wilkins). For example, the active compound can be mixed with a carrier, or diluted by a carrier, or enclosed within a carrier, which may be in the form of an ampule, capsule, or sachet. When the carrier serves as a diluent, it may be a solid, semi-solid, or liquid material that acts as a vehicle, excipient, or medium for the active compound.

The pharmaceutical compositions may be in conventional forms, for example, capsules, tablets, solutions, suspensions, injectables or products for topical application. Further, the pharmaceutical composition of the present invention may be formulated so as to provide desired release profile.

The route of administration may be any route which effectively transports the active compound to the appropriate or desired site of action. Suitable routes of administration include, but are not limited to, oral, nasal, pulmonary, buccal, subdermal, intradermal, transdermal, parenteral, rectal, depot, subcutaneous, intravenous, intraurethral, intramuscular, intranasal, ophthalmic (such as with an ophthalmic solution) or topical (such as with a topical ointment). The oral route is specifically suitable.

Solid oral formulations include, but are not limited to, tablets, capsules (soft or hard gelatin), dragees (containing the active ingredient in powder or pellet form), troches and lozenges. Tablets, dragees, or capsules having talc and/or a carbohydrate carrier or binder or the like are particularly suitable for oral application. Exemplary carriers for tablets, dragees, or capsules include lactose, cornstarch, and/or potato starch. A syrup or elixir can be used in cases where a sweetened vehicle can be employed.

A typical tablet that may be prepared by conventional tableting techniques.

Liquid formulations include, but are not limited to, syrups, emulsions, soft gelatin and sterile injectable liquids, such as aqueous or non-aqueous liquid suspensions or solutions.

For parenteral application, particularly suitable are injectable solutions or suspensions, specifically aqueous solutions with the active compound dissolved in polyhydroxylated castor oil.

### Methods of Screening

Antiviral HIV activity and cytotoxicity of compounds present invention can be measured in parallel by following the methods published in the literature.

The cytotoxic effect of compounds can be analyzed by measuring the proliferation of cells using the 3-(4,5-Dimethylthiazol-2-yl)-2,5-diphenyl tetrazlium bromide (MTT) staining. Cells (5×10³ cells/well) will be incubated in in 96 well plates in the presence or absence of compounds. At the end of treatment, 20!J.1 of MTT (5 mg/ml in PBS) will be added to each well and incubated for an additional 4 hours at 37° C. The purple-blue MTT formazan precipitate will be dissolved in a triplex reagent containing 10% SDS, 5% isobutanol and 10 mmol/lit HCl. The activity of mitochondria, reflecting cellular growth and viability, will be evaluated by measuring the optical density at 570 nm on micro titer plate.

Action of compounds on replication of HIV in Sup-T1 cells can be determined by the method published by Roda Rani et al., 2006 (Archives of Biochemistry and Biophysics, Volume 456, Issue 1, 1 Dec. 2006, Pages 79-92).

Briefly, 1×10⁶ Sup-T1 cells with 100% cell viability will be seeded in RPMI 1640, 0.1% FBS four 12 well plates. Increasing concentrations of Epap-1 peptides will be added to the cells and will be infected with HIV1_{93 IN 101} each at final concentration of virus equivalent to 2 ng of p24 per ml. The infected cells will be incubated at 3 7° C. and 5% CO₂ incubator for 2 hours. After 2 hours the cells will be pelleted at 350 g for 10 minutes, supernatant will be discarded and cell will be held with RPMI 1640 containing 10% FBS. The cells will be resuspended in the same medium with increasing concentrations of Epap-1 peptides and will be incubated for 96 hours. The cells will be supplemented with peptides at every 24 hours. The supernatants will be collected after 96 hours and analyzed using P24 antigen capture assay kit (SAIC Fredrick). The infection in the absence of Epap-1 will be considered to be 0% inhibition Azidothymidine (AZT) will be taken as positive control.

Action of compound on virus entry and quantification of virus entered can be done in terms of GFP expression by the following the methods published J. Virol. 72, 6988 (1998) by in Cecilia et al., and Analytical Biochemistry Volume 360, Issue 2, 15 Jan. 2007, Pages 315-317 (Dyavar S. Ravi and Debashis Mitra).

Briefly, cells will be seeded in to wells of 24 well plates 1 day prior to the experiment. The cells will be transfected with Tat-reporter. The virus inoculum will be adjusted to 1,000-4,000 TCID 50/ml in assay medium (DMEM, 10% FCS, glutamine and antibiotics), 50 μl aliquots will be incubated with serial dilutions of compounds (50 μl) for 1 hour at 37° C. The reporter expression will be quantified at appropriate time calculated inhibitory doses referrers to the concentration of these agents in this preincubation mixture.

Other relevant references useful for screening antiviral HIV activity are: Averett, D. R. 1989. Anti-HIV compound assessment by two novel high capacity assays. J. Virol. Methods 23: 263-276; Schwartz, 0., et al. 1998; A rapid and simple colorimeric test for the study of anti HIV agents. AIDS Res. and Human Retroviruses, 4(6):441-447; Daluge, S. M., et al. 1994. 5-Chloro-2′,3′-deoxy-3′fluorouridine (935U83), a selective anti human immunodeficiency virus agent with an improved metabolic and toxicological profile; Antimicro. Agents and Chemotherapy, 38(7): 1590-1603; H. Mitsuya and S. Border, Inhibition of the in vitro infectivity and cytopathic effect of human T-lymphotropic virus type lymphadenopathy-associated virus (HLTV-111/LAV) by 2,3′-dideoxynucleosides, Proc. Natl. Acad. Sci. USA, 83, 1911-15(1986); Pennington et al., Peptides 1990; Meek T. D et al., Inhibition of HIV-1 protease in infected T-limphocytes by synthetic peptide analogues, Nature, 343, p 90 (1990); Weislow et al., J. Natl. Cancer Inst. 81, 577-586, 1989; T. Mimoto et al., J. Med. Chern., 42, 1789-1802, 1999; Uckun et al 1998, Antimicrobial Agents and Chemotherapy 42:383; for P24 antigen assay Erice et al., 1993, Antimicrob. Ag. Chemotherapy 37: 385-383; Koyanagi et al., Int. J. Cancer, 36, 445-451, 1985; Balzarini et al. AIDS (1991), 5, 21-28; Connor et al., Journal of virology, 1996, 70, 5306-5311; Popik et al., Journal of virology, 2002, 76, 4709-4722; Harrigton et al., Journal of Virology Methods, 2000, 88, 111-115; Roos et al., Virology 2000, 273, 307-315; Fedyuk N. V. et al; Problems of Virology 1992, (3)P135; Mosmann T, December 1983, Journal of immunological methods, 65 (1-2), 55-63; SPC Cole, cancer chemotherapy and Pharmacology, 1986, 17, 259-263, Antiviral methods and protocols (Eds: D Kinchington and R. F. Schinazi) Humana Press Inc., 2000, HIV protocols (Eds: N. L. Michael and J. H. Kim) Humana Press Inc, 1999, DAIDS Virology manual from HIV laboratories, Publication NIH-97-3838, 1997, 4. HIV-1 p24 antigen capture assay, enzyme immunoassay for detection of Human immunodeficiency Virus Type 1 (HIV-1) p24 in tissue culture media—Advanced bio science laboratories, Inc kit procedure.

### Methods of Treatment

The present invention provides compounds and pharmaceutical formulations thereof that are useful in the treatment of diseases, conditions and/or disorders mediated by viral infections. The connection between therapeutic effect and antiviral is illustrated. For example, PCT publication Nos. WO 01//07646, WO 01/65957, or WO 03/037908; US publication Nos. U.S. Pat. No. 4,598,095 or US 2002/0068757; EP publication Nos. EP 0989862 or EP 0724650; Bioorganic & Medicinal Chemistry Letters, 16, (6), 1712-1715, 2006; and references cited therein, all of which are incorporated herein by reference in their entirety and for the purpose stated.

The present invention further provides a method of treating a disease, condition and/or disorder mediated by viral infections in a subject in need thereof by administering to the subject a therapeutically effective amount of a compound or a pharmaceutical composition of the present invention.

Diseases, conditions, and/or disorders that are mediated by viral infections are believed to include, but are not limited to, HIV infection, HBV infection, HCV infection, a retroviral infection genetically related to HIV, AIDS, inflammatory disease, respiratory disorders (including adult respiratory distress syndrome (ARDS), bronchitis, chronic bronchitis, chronic obstructive pulmonary disease, cystic fibrosis, asthma, emphysema, rhinitis and chronic sinusitis), inflammatory bowel disease (including Crohn's disease and ulcerative colitis), multiple sclerosis, rheumatoid arthritis, graft rejection (in particular but not limited to kidney and lung allografts), endometriosis, type I diabetes, renal diseases, chronic pancreatitis, inflammatory lung conditions, chronic heart failure and bacterial infections (in particular but not limited to tuberculosis).

The compounds of the present invention can obtain more advantageous effects than additive effects in the prevention or treatment of the above diseases when using suitably in combination with the available drugs. Also, the administration dose can be decreased in comparison with administration of either drug alone, or adverse effects of co administrated drugs other than antiviral can be avoided or declined.

### Methods of Preparation

The compounds described herein may be prepared by techniques known in the art. In addition, the compounds described herein may be prepared by following the reaction sequence as depicted in below schemes. Further, in the following schemes, where specific bases, acids, reagents, solvents, coupling agents, etc., are mentioned, it is understood that other bases, acids, reagents, solvents, coupling agents etc., known in the art may also be used and are therefore included within the present invention. Variations in reaction conditions, for example, temperature and/or duration of the reaction, which may be used as known in the art, are also within the scope of the present invention. All the stereoisomers of the compounds in these schemes, unless otherwise specified, are also encompassed within the scope of this invention.

Compounds of the present invention can be synthesized from naturally occurring Betulinic acid. Key intermediates required for synthesizing analogues are either commercially available or can be prepared by the methods published in the literature. For example, the key intermediates in the present invention were prepared by modifying the procedures published in Journal of organic chemistry 2010, 75, 1285-1288; Journal of organic chemistry 2000, 65, 3934-3940; Tetrahedron: asymmetry 2008, 19, 302-308; or Tetrahedron: asymmetry 2003, 14, 217-223.

Another embodiment of the present invention provides process for preparation of the compounds of general formula (I) are set forth in the below generalized schemes. One of skilled in the art will recognize that below generalised schemes can be adapted to produce the compounds of general formula (I) and pharmaceutically acceptable salts according to the present invention. Wherein all symbols/variables are as defined earlier unless otherwise stated.

### General Synthetic Procedures:

### Synthetic Scheme for Intermediate:

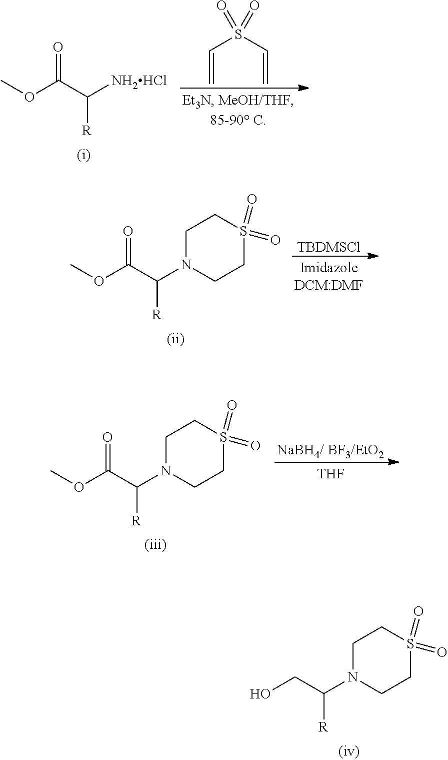

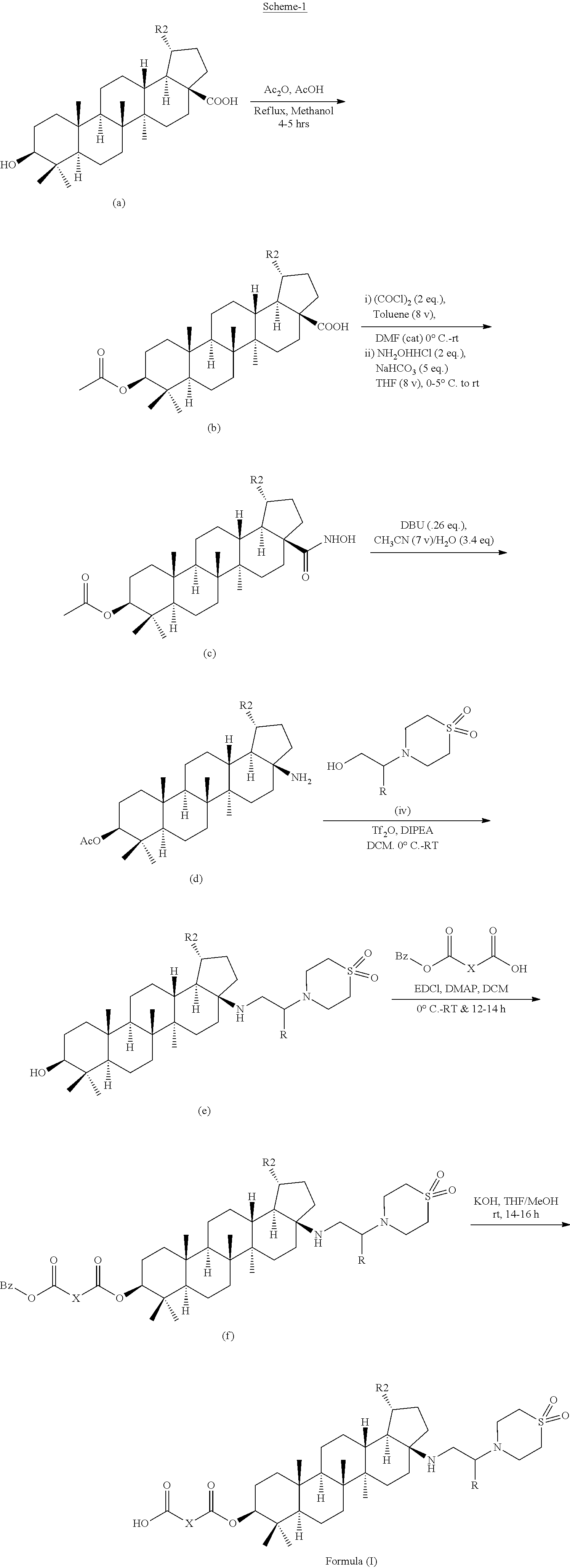

Synthetic Scheme for Intermediate:

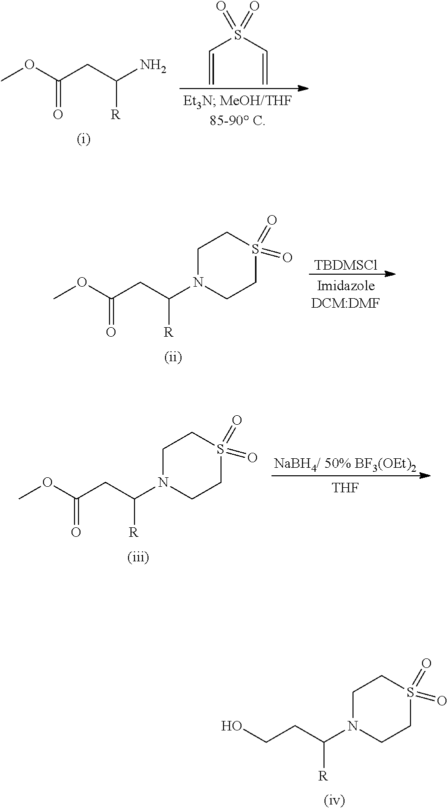

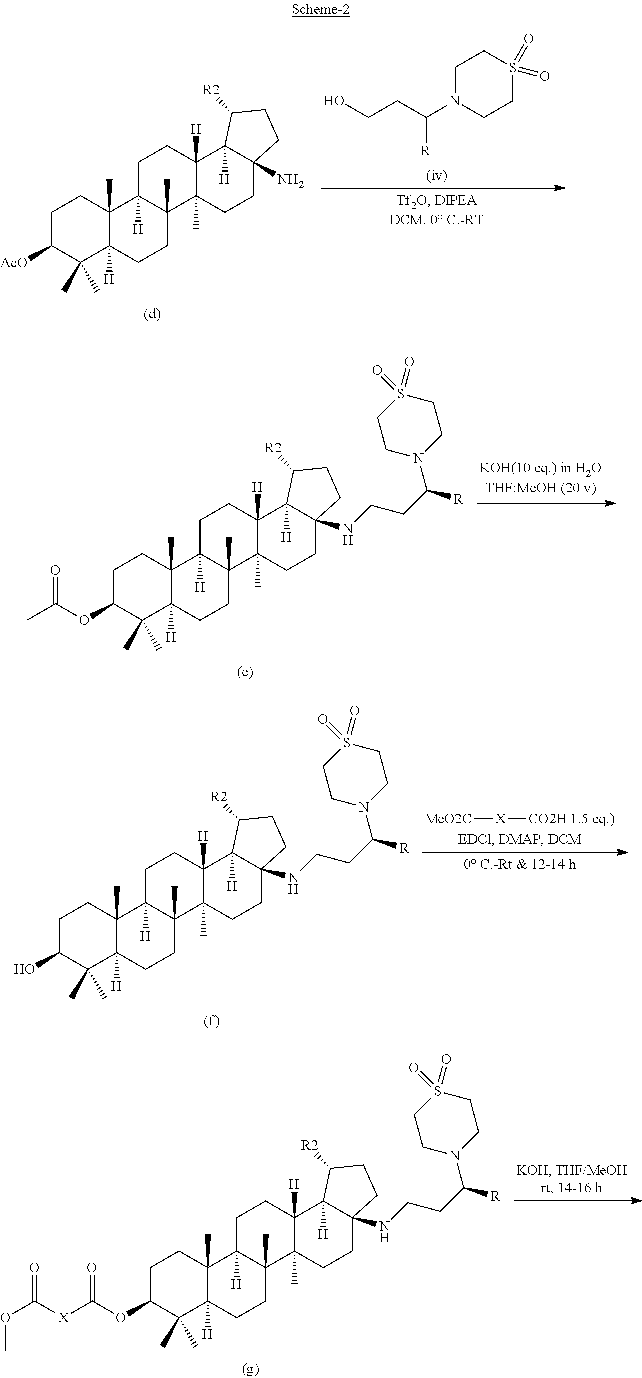

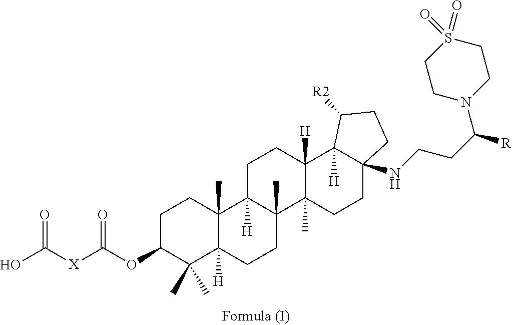

### EXPERIMENTAL

The present invention is further illustrated by the following examples, which are not to be construed in any way as imposing limitations upon the scope of this disclosure, but rather are intended to be illustrative only. On the contrary, it is to be clearly understood that resort may be had to various other embodiments, modifications, and equivalents thereof which, after reading the description herein, may suggest one of ordinary skill in the art without departing from the spirit of the present invention. Thus, the skilled artisan will appreciate how the experiments and examples may be further implemented as disclosed by variously altering the following examples, substituents, reagents, or conditions.

### Intermediates

### Intermediate 1: Preparation of 1-((1R,3aS,5aR,5bR,7aR,9S,11aR,11bR,13aR,13bR)-3a-amino-5a,5b,8,8,11a-pentamethyl-1-(1-methylcyclopropyl)icosahydro-1H-cyclopenta[a]chrysen-9-yl) 3-benzyl (1S,3R)-2,2-dimethylcyclobutane-1,3-dicarboxylate

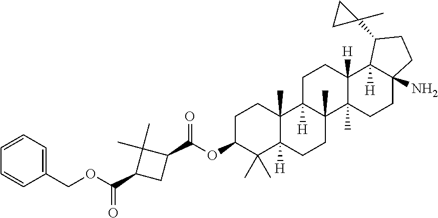

### Step 1: Synthesis of (1R,3aS,5aR,5bR,7aR,9S,11aR,11bR,13aR,13bR)-9-acetoxy-5a,5b,8,8,11a-pentamethyl-1-(prop-1-en-2-yl)icosahydro-3aH-cyclopenta[a]chrysene-3a-carboxylic acid

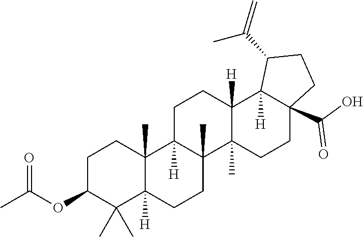

To Betulinic acid (200 g, 0.438 moles, 1.0 eq) in RB flask, acetic acid (1600 ml) and acetic anhydride (173 g, 1.696 moles, 3.87 eq) were added. The reaction mixture was stirred and heated to 130-140° C. for around 4 hours. The progress of the reaction monitored by TLC, indicated starting material was completed. Acetic acid was distilled out approximately 1100 ml under vacuum. The flask was cooled to room temperature, methanol (1400 ml) was slowly added and stir at room temperature for 12-16 hours. The solid was filtered, washed with methanol (200 ml) and dried under vacuum (50-55° C.) to obtain the title compound as a white solid (182 g, 83.32% yield).

### Step 2: Synthesis of benzyl (1R,3aS,5aR,5bR,7aR,9S,11aR,11bR,13aR,13bR)-9-acetoxy-5a,5b,8,8,11a-pentamethyl-1-(prop-1-en-2-yl)icosahydro-3aH-cyclopenta[a]chrysene-3a-carboxylate

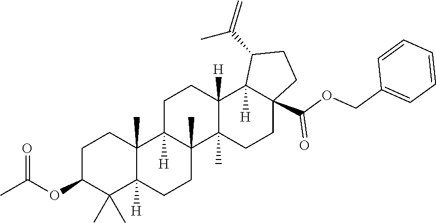

To a stirred solution of (1R,3aS,5aR,5bR,7aR,9S,11aR,11bR,13aR,13bR)-9-acetoxy-5a,5b,8,8,11a-pentamethyl-1-(prop-1-en-2-yl)icosahydro-3aH-cyclopenta[a]chrysene-3a-carboxylic acid (step 1, 180 g, 0.361 moles, 1.0 eq) in acetonitrile (1800 ml) was added potassium carbonate (99.75 g, 0.721 moles, 2.0 eq). The reaction mixture was stirred at room temperature, Benzyl bromide (42.5 ml, 0.361 moles, 1.0 eq) was added drop wise for 30 minutes. The reaction mixture was stirred and heated to reflux for around 6 hours. The progress of the reaction monitored by TLC, indicated starting material was completed. The reaction mixture was cooled to room temperature, water (2700 ml) was added and stirred for 2 hours. Ethyl acetate (2700 ml) was added and stirred for 30 minutes. Organic layer was separated and aqueous layer was again extracted twice with ethyl acetate (2×900 ml). The combined organic layers were washed with water (1800 ml) and brine solution (1800 ml). The organic layer was dried over sodium sulphate, filtered and concentrated under reduced pressure. The crude compound was purified by treating with methanol (720 ml), stirred and heated to 60-65° C. for 30 minutes. Cool the reaction mixture to room temperature and stirred for 2 hours. The mixture was filtered, washed with methanol (180 ml) and dried under vacuum (55-60° C.) to obtain the title compound (180 g, 84.7% yield) as a solid.

### Step 3: Synthesis of benzyl (1R,3aS,5aR,5bR,7aR,9S,11aR,11bR,13aR,13bR)-9-acetoxy-5a,5b,8,8,11a-pentamethyl-1-(1-methylcyclopropyl)icosahydro-3aH-cyclopenta[a]chrysene-3a-carboxylate

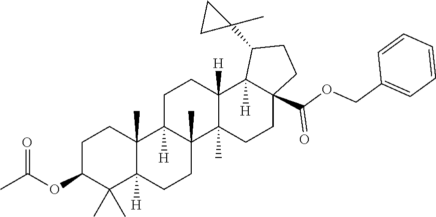

To a stirred solution of benzyl (1R,3aS,5aR,5bR,7aR,9S,11aR,11bR,13aR,13bR)-9-acetoxy-5a,5b,8,8,11a-pentamethyl-1-(prop-1-en-2-yl)icosahydro-3aH-cyclopenta[a]chrysene-3a-carboxylate (step 2, 120 g, 0.204 moles, 1.0 eq) in DCM (1200 ml) at −25 to −35° C. under nitrogen atmosphere was added Diethyl Zinc (75.61 g, 408 ml, 0.612 mol, 3.0 eq, 1.5M in Toluene). The reaction mixture was stirred at −25 to −35° C. for 1 hour. Diiodomethane (81.5 ml, 1.02 moles, 5.0 eq) was slowly added drop wise at −25 to −35° C. for around 45 minutes. The reaction mixture was stirred at −25 to −35° C. for around 3 hours. The reaction mixture was slowly warmed to room temperature and stirred for around 14 hours. TLC indicated starting material was completed and the desired product was observed. The reaction mixture was cooled to 0-5° C., quenched with saturated ammonium chloride solution (600 ml), 1N HCl (240 ml) was slowly added to adjust the pH (3 to 4) and stirred for 30 minutes. The organic layer was separated and the aqueous layer was extracted with DCM (600 ml). The combined organic layers were washed with 10% sodium bicarbonate solution, water (1800 ml) and brine solution (1800 ml). The organic layer was dried over sodium sulphate, filtered and concentrated under reduced pressure. To this obtained solid, Diisopropyl ether (360 ml) was added and stirred at room temperature for 3 hours. The solid was filtered, washed with diisopropyl ether (120 ml) and dried under vacuum (55-60° C.) to obtain the title compound (100 g, 81.4% yield) as a white solid.

### Step 4: Synthesis of (1R,3aS,5aR,5bR,7aR,9S,11aR,11bR,13aR,13bR)-9-acetoxy-5a,5b,8,8,11a-pentamethyl-1-(1-methylcyclopropyl)icosahydro-3aH-cyclopenta[a]chrysene-3a-carboxylic acid

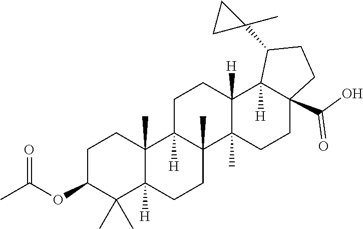

To a suspension of 10% Pd/C (15 g, 50% wet) in ethyl acetate (350 ml) was added benzyl (1R,3aS,5aR,5bR,7aR,9S,11aR,11bR,13aR,13bR)-9-acetoxy-5a,5b,8,8,11a pentamethyl-1-(1-methylcyclopropyl)icosahydro-3aH-cyclopenta[a]chrysene-3a-carboxylate (step 3, 35 g, 58.13 mmol, 1.0 eq) dissolved in methanol (200 ml). The reaction mixture was hydrogenated in parr shaker apparatus at 50 psi pressure for 3 hours. TLC indicates consumption of starting material. The reaction mixture was filtered through celite pad and the filtrate was evaporated under reduced pressure to obtain the title compound (30 g) as a solid. ¹H NMR (300 MHz, CDCl₃): δ ppm 4.51-4.46 (m, 1H), 2.25-2.21 (m, 1H), 2.13-0.78 (m, 24H), 2.04 (s, 3H), 0.98 (s, 3H), 0.91 (s, 6H), 0.86 (s, 3H), 0.85 (s, 3H), 0.84 (s, 3H), 0.38-0.34 (m, 2H), 0.27-0.24 (m, 2H); ESI-MS: m/z 511.45 (M−H)⁻.

### Step 5: Synthesis of (1R,3aS,5aR,5bR,7aR,9S,11aR,11bR,13aR,13bR)-3a-isocyanato-5a,5b,8,8,11a-pentamethyl-1-(1-methylcyclopropyl)icosahydro-1H-cyclopenta[a]chrysen-9-yl acetate

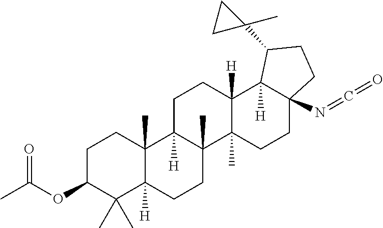

To a stirred solution of (1R,3aS,5aR,5bR,7aR,9S,11aR,11bR,13aR,13bR)-9-acetoxy-5a,5b,8,8,11a-pentamethyl-1-(1-methylcyclopropyl)icosahydro-3aH-cyclopenta[a]chrysene-3a-carboxylic acid (step 4, 20 g, 39.00 mmol, 1.0 eq) in Toluene (200 ml) was added Diphenylphosphoryl azide (22.36 ml, 97.50 mmol, 2.5 eq) and triethylamine (13.70 ml, 97.50 mmol, 2.5 eq). The reaction mixture was refluxed for 1 hour. TLC indicated starting material was consumed and the desired product was observed. The reaction mixture was evaporated under reduced pressure, diluted with water (250 ml) and extracted with DCM (2×200 ml). The combined organic layer was dried over Na₂SO₄, filtered and concentrated under reduced pressure. The residue was triturated with methanol (100 ml), filtered and dried under vacuum to obtain the title compound (19 g, 95.5% yield) as an off-white solid. ¹H NMR (300 MHz, CDCl₃): δ ppm 4.45 (m, 1H), 2.04 (s, 3H), 2.03-0.78 (m, 25H), 1.04 (s, 3H), 0.94 (s, 3H), 0.91 (s, 3H), 0.88 (s, 3H), 0.85 (s, 3H), 0.84 (s, 3H), 0.38-0.26 (m, 4H).

### Step 6: Synthesis of (1R,3aS,5aR,5bR,7aR,9S,11aR,11bR,13aR,13bR)-3a-amino-5a,5b,8,8,11a-pentamethyl-1-(1-methylcyclopropyl)icosahydro-1H-cyclopenta[a]chrysen-9-ol hydrochloride

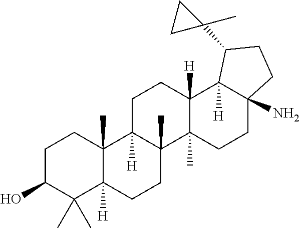

To a stirred solution of (1R,3aS,5aR,5bR,7aR,9S,11aR,11bR,13aR,13bR)-3a-isocyanato-5a,5b,8,8,11a-pentamethyl-1-(1-methylcyclopropyl)icosahydro-1H-cyclopenta[a]chrysen-9-yl acetate (step 5, 19 g, 37.27 mmol, 1.0 eq) in 1,4-dioxane (190 ml) at 0° C. was added concentrated HCl (57 ml). The reaction mixture was stirred at 50-60° C. for overnight. TLC indicated starting material was consumed and the desired product was observed. The reaction mixture was evaporated under reduced pressure and co-distilled with acetonitrile (100 ml) to obtain the title compound (20 g) as a solid, which is used as such for next step without further purification. ES-MS: m/z 442.20 (M+H)⁺.

### Step 7: Synthesis of tert-butyl ((1R,3aS,5aR,5bR,7aR,9S,11aR,11bR,13aR,13bR)-9-hydroxy-5a,5b,8,8,11a-pentamethyl-1-(1-methylcyclopropyl)icosahydro-3aH-cyclopenta[a]chrysen-3a-yl)carbamate

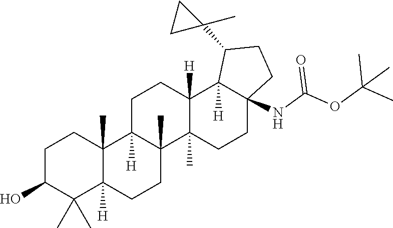

To a stirred solution of (1R,3aS,5aR,5bR,7aR,9S,11aR,11bR,13aR,13bR)-3a-amino-5a,5b,8,8,11a-pentamethyl-1-(1-methylcyclopropyl)icosahydro-1H-cyclopenta[a]chrysen-9-ol hydrochloride (step 6, 20 g, 41.823 mmol, 1.0 eq) in 1,4-dioxane (200 ml) at 0° C. was added saturated sodium bicarbonate solution (100 ml) and di-tert-butyldicarbonate (14.41 ml, 62.734 mmol, 1.5 eq). The reaction mixture was stirred at room temperature for overnight. TLC indicated starting material was consumed and the desired product was observed. The reaction mixture was evaporated under reduced pressure, diluted with water and extracted with DCM (2×200 ml). The combined organic layer was dried over Na₂SO₄, filtered and concentrated under reduced pressure. The residue was purified by silica gel column chromatography using 10% Ethyl acetate in hexane as an eluent to obtain the title compound (9 g, 44.57% yield over two steps) as an off-white solid. ¹H NMR (300 MHz, CDCl₃): δ ppm 3.23-3.16 (m, 1H), 2.54-2.51 (m, 1H), 2.42-2.25 (m, 2H), 2.0-1.80 (m, 2H), 1.73-0.67 (m, 20H), 1.42 (s, 9H), 1.01 (s, 3H), 0.97 (s, 6H), 0.91 (s, 3H), 0.85 (s, 3H), 0.77 (s, 3H), 0.40-0.25 (m, 4H); ES-MS: m/z 564.17 (M+Na)⁺.

### Step 8: Synthesis of 1-benzyl 3-((1R,3aS,5aR,5bR,7aR,9S,11aR,11bR,13aR,13bR)-3a-((tert-butoxycarbonyl)amino)-5a,5b,8,8,11a-pentamethyl-1-(1-methylcyclopropyl)icosahydro-1H-cyclopenta[a]chrysen-9-yl) (1R,3S)-2,2-dimethylcyclobutane-1,3-dicarboxylate

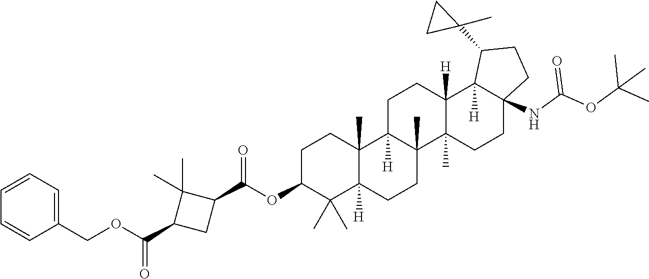

To a stirred solution of tert-butyl ((1R,3aS,5aR,5bR,7aR,9S,11aR,11bR,13aR,13bR)-9-hydroxy-5a,5b,8,8,11a-pentamethyl-1-(1-methylcyclopropyl)icosahydro-3aH-cyclopenta[a]chrysen-3a-yl)carbamate (step 7, 9 g, 16.609 mmol, 1.0 eq) in Dichloromethane (90 ml) was added (1S,3R)-3-((benzyloxy)carbonyl)-2,2-dimethylcyclobutane-1-carboxylic acid (prepared according to the procedure described in WO2011/007230A2, 8.71 g, 33.21 mmol, 2.0 eq), 1-(3-Dimethylaminopropyl)-3-ethylcarbodiimide Hydrochloride (EDC.HCl) (9.55 g, 49.82 mmol, 3.0 eq) and 4-(Dimethylamino)pyridine (0.81 g, 6.643 mmol, 0.4 eq). The reaction mixture was stirred at room temperature for overnight. TLC indicated starting material was consumed and the desired product was observed. The reaction mixture was diluted with water (100 ml) and extracted with dichloromethane (2×100 ml). The combined organic layer was dried over Na₂SO₄, filtered and concentrated under reduced pressure. The crude compound was purified by silica gel column chromatography using 10% ethyl acetate:hexane as an eluent to obtain the title compound (11 g, 84.29% yield) as an off-white solid. ¹H NMR (300 MHz, CDCl₃): δ ppm 7.34 (m, 5H), 5.15, 5.09 (ABq, J_{AB}=12.3 Hz, 2H), 4.44 (dd, J=11.1, 4.5 Hz, 1H), 4.17 (s, 1H), 2.84-2.73 (m, 2H), 2.69-2.58 (m, 1H), 2.57-2.51 (m, 1H), 2.42-2.23 (m, 2H), 2.07-1.81 (m, 3H), 1.72-0.76 (m, 20H), 1.42 (s, 9H), 1.34 (s, 3H), 1.0 (s, 3H), 0.96 (s, 6H), 0.91 (s, 3H), 0.89 (s, 3H), 0.87 (s, 3H), 0.85 (s, 3H), 0.40-0.25 (m, 4H); ES-MS: m/z 786.47 (M+H)⁺.

### Step 9: Synthesis of 1-((R,3aS,5aR,5bR,7aR,9S,11aR,11bR,13aR,13bR)-3a-amino-5a,5b,8,8,11a-pentamethyl-1-(1-methylcyclopropyl)icosahydro-1H-cyclopenta[a]chrysen-9-yl) 3-benzyl (1S,3R)-2,2-dimethylcyclobutane-1, 3-dicarboxylate

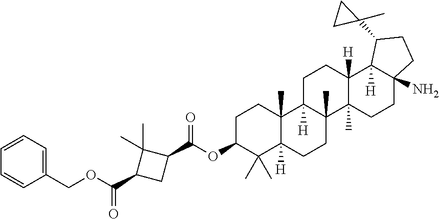

To a stirred solution of 1-benzyl 3-((1R,3aS,5aR,5bR,7aR,9S,11aR,11bR,13aR,13bR)-3a-((tert-butoxycarbonyl)amino)-5a,5b,8,8,11a-pentamethyl-1-(1-methylcyclopropyl) icosahydro-1H-cyclopenta[a]chrysen-9-yl) (1R,3S)-2,2-dimethylcyclobutane-1,3-dicarboxylate (step 8, 11 g, 13.99 mmol, 1.0 eq) in 1,4-dioxane (11 ml) at 0° C. was added 4N HCl in 1,4-dioxane (33 ml). The reaction mixture was stirred at room temperature for overnight. TLC indicated starting material was consumed and the desired product was observed. The reaction mixture was evaporated under reduced pressure, residue was basified with saturated sodium bicarbonate solution and extracted with dichloromethane (3×100 ml). The combined organic layer was washed with water, dried over Na₂SO₄, filtered and evaporated under reduced pressure to obtain the title compound (9 g, 93.84% yield) as an off-white solid. ¹H NMR (300 MHz, CDCl₃): δ ppm 7.34 (m, 5H), 5.15, 5.09 (ABq, J_{AB}=12.3 Hz, 2H), 4.45 (m, 1H), 2.81-2.73 (m, 2H), 2.65-2.62 (m, 1H), 2.08-1.88 (m, 3H), 1.74-0.78 (m, 23H), 1.34 (s, 3H), 1.03 (s, 3H), 0.96 (s, 6H), 0.91 (s, 3H), 0.87 (s, 3H), 0.85 (s, 3H), 0.84 (s, 3H), 0.37-0.22 (m, 4H); ES-MS: m/z 686.45 (M+H)⁺.

### Intermediate 2: Synthesis of (1S,3aS,5aR,5bR,7aR,9S,11aR,11bR,13aR,13bR)-9-acetoxy-1-isopropyl-5a,5b,8,8,11a-pentamethylicosahydro-3aH-cyclopenta[a]chrysene-3a-carboxylic acid

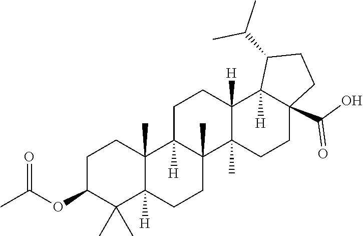

To a suspension of 10% Pd/C (15 g, 50% wet) in ethyl acetate (350 ml) was added (1R,3aS,5aR,5bR,7aR,9S,11aR,11bR,13aR,13bR)-9-acetoxy-5a,5b,8,8,11a-pentamethyl-1-(prop-1-en-2-yl)icosahydro-3aH-cyclopenta[a]chrysene-3a-carboxylic acid (1 mmol) dissolved in methanol (200 ml). The reaction mixture was hydrogenated in parr shaker apparatus at 50 psi pressure for 3 hours. TLC indicates consumption of starting material. The reaction mixture was filtered through celite pad and the filtrate was evaporated under reduced pressure to obtain the title compound in quantitative yield as a solid.

### Intermediate 3: Preparation of methyl (R)-2-(1,1-dioxidothiomorpholino)-3-hydroxy propanoate

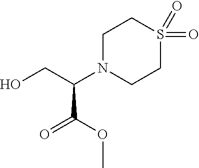

To a stirred solution of methyl D-serinate hydrochloride (4.0 g, 25.70 mmol, 1.0 eq) in 1,4-dioxane (20 ml) and ethanol (20 ml) were added triethylamine (14.34 ml, 102.8 mmol, 4.0 eq) followed by divinyl sulfone (5.15 ml, 51.4 mmol, 2.0 eq). The reaction mixture was heated to 85° C. for 3 hours. TLC indicated starting material was completed and the desired product was observed. The reaction mixture was cooled to room temperature and concentrated under reduced pressure. The crude compound was purified by silica gel column chromatography using 5-10% methanol in dichloromethane gradient to obtain the title compound (5.8 g, 95% yield) as a white solid. ¹H NMR (300 MHz, CDCl₃): δ ppm 3.90-3.84 (m, 1H), 3.81-3.73 (m, 1H), 3.76 (s, 3H), 3.54 (dd, J=8.1, 6.0 Hz, 1H), 3.40-3.30 (m, 2H), 3.17-3.13 (m, 1H), 3.09-3.06 (m, 5H), 2.48 (s, 1H).

### Intermediate 4: Preparation of 4-((2R,3R)-3-((tert-butyldimethylsilyl)oxy)-1-hydroxybutan-2-yl)thiomorpholine 1,1-dioxide

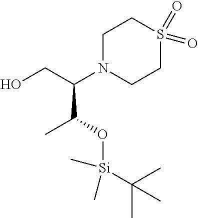 [SMILES: C[C@@H](O[Si](C)(C)C(C)(C)C)[C@@H](CO)N1CCS(=O)(=O)CC1]

### Step 1: Synthesis of methyl L-threoninate

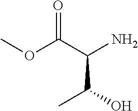

To a stirred solution of L-threonine (10 g, 83.94 mmol, 1.0 eq) in methanol (100 ml) at 0° C. was added thionyl chloride (18.38 ml, 251.84 mmol, 3.0 eq). The reaction mixture was heated to reflux for about 3 hours. TLC indicated starting material was completed and the desired product was observed. The reaction mixture was evaporated under reduced pressure, ethyl acetate (100 ml) and triethylamine (35.39 ml, 251.84 mmol, 3.0 eq) were added to the reaction mixture, precipitate was formed. The precipitates formed were collected by filtration and the filtrate was evaporated under reduced pressure to obtain the title compound (8 g) as oil. The obtained compound was used as such for next step without further purification. ¹H NMR (300 MHz, DMSO-d6): δ ppm 4.62 (s, 1H), 3.85-3.82 (m, 1H), 3.61 (s, 3H), 3.16 (d, J=3.9 Hz, 1H), 1.08 (d, J=6.3 Hz, 3H); ES-MS: m/z 134.07 (M+H)⁺.

### Step 2: Synthesis of methyl (2S,3R)-2-(1,1-dioxidothiomorpholino)-3-hydroxybutanoate

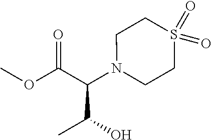 [SMILES: COC(=O)[C@H]([C@@H](C)O)N1CCS(=O)(=O)CC1]

To a stirred solution of methyl L-threoninate (step 1, 2.5 g, 18.77 mmol, 1.0 eq) in ethanol (25 ml) and 1,4-dioxane (25 ml) was added triethylamine (7.91 ml, 56.31 mmol, 3.0 eq) and divinyl sulfone (5.61 ml, 37.55 mmol, 2.0 eq). The reaction mixture was heated to reflux for about 3 hours. TLC indicated starting material was consumed and the desired product was observed. The reaction mixture was evaporated under reduced pressure. The residue was purified by silica gel column chromatography using 2% methanol in DCM as an eluent to obtain the title compound (4 g, 85.1% yield) as a solid. ¹H NMR (300 MHz, CDCl₃): δ ppm 4.13-3.96 (m, 1H), 3.76 (s, 3H), 3.42-2.90 (m, 9H), 1.21 (d, J=6.0 Hz, 3H); ES-MS: m/z 252.26 (M+H)⁺.

### Step 3: Synthesis of methyl (2S,3R)-3-((tert-butyldimethylsilyl)oxy)-2-(1,1-dioxido thiomorpholino)butanoate

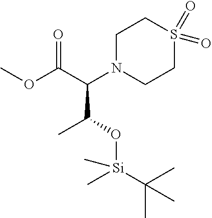 [SMILES: COC(=O)[C@H]([C@@H](C)O[Si](C)(C)C(C)(C)C)N1CCS(=O)(=O)CC1]

To a stirred solution of methyl (2S,3R)-2-(1,1-dioxidothiomorpholino)-3-hydroxybutanoate (step 2, 1.5 g, 5.976 mmol, 1.0 eq) in DCM (15 ml) and DMF (10 ml) was added imidazole (0.813 g, 11.952 mmol, 2.0 eq) and tert-Butyldimethylsilyl chloride (1.35 g, 8.964 mmol, 1.5 eq). The reaction mixture was stirred at room temperature for overnight. TLC indicated starting material was consumed and the desired product was observed. The reaction mixture was diluted with water (20 ml) and extracted with DCM (2×30 ml). The combined organic layer was separated, dried over Na₂SO₄, filtered and concentrated under reduced pressure. The residue was purified by silica gel column chromatography using 1% methanol in DCM as an eluent to obtain the title compound (0.6 g, 27.52% yield) as an off-white solid. ¹H NMR (300 MHz, CDCl₃): δ ppm 4.39 (m, 1H), 3.73 (s, 3H), 3.64-3.56 (m, 2H), 3.30-3.24 (m, 3H), 3.06-3.03 (m, 4H), 1.26 (d, J=6.3 Hz, 3H), 0.86 (s, 9H), 0.08 (s, 3H), 0.04 (s, 3H); ES-MS: m/z 388.10 (M+Na)⁺.

### Step 4: Synthesis of 4-((2R,3R)-3-((tert-butyldimethylsilyl)oxy)-1-hydroxybutan-2-yl) thiomorpholine 1,1-dioxide

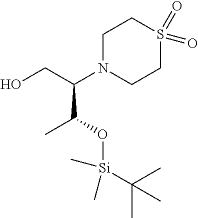 [SMILES: C[C@@H](O[Si](C)(C)C(C)(C)C)[C@@H](CO)N1CCS(=O)(=O)CC1]

To a stirred solution of methyl (2S,3R)-3-((tert-butyldimethylsilyl)oxy)-2-(1,1-dioxidothiomorpholino)butanoate (step 3, 0.6 g, 1.641 mmol, 1.0 eq) in DCM (12 ml) at −78° C. was added DIBAL-H (6.5 ml, 9.847 mmol, 6.0 eq, 1.5M in toluene). The reaction mixture was stirred at same temperature for about 4 hours. TLC indicated starting material was consumed and the desired product was observed. The reaction mixture was quenched with saturated ammonium chloride solution and extracted with ethyl acetate (2×30 ml). The organic layer was washed with water, dried over Na₂SO₄, filtered and concentrated under reduced pressure. The residue was purified by silica gel column chromatography using 2% methanol in DCM as an eluent to obtain the title compound (0.442 g, 79.92% yield) as an off-white solid. ¹H NMR (300 MHz, CDCl₃): δ ppm 4.11-4.03 (m, 1H), 3.71-3.64 (m, 2H), 3.55-3.39 (m, 4H), 3.19-3.08 (m, 4H), 2.81-2.74 (m, 1H), 2.49 (s, 1H), 1.30 (d, J=6.0 Hz, 3H), 1.0 (s, 9H), 0.20 (s, 6H); ESI-MS: m/z 360.05 (M+Na)⁺.

### Intermediate 5: Synthesis of 4-(1-((tert-butyldimethylsilyl)oxy)-4-hydroxybutan-2-yl)thiomorpholine L1-dioxide

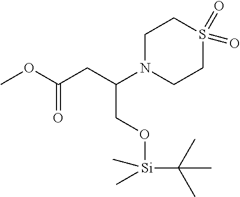 [SMILES: COC(=O)CC(CO[Si](C)(C)C(C)(C)C)N1CCS(=O)(=O)CC1]

### Step 1: Synthesis of methyl 3-amino-4-hydroxybutanoic acid

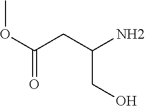

To a stirred solution of 3-amino-4-hydroxybutanoic acid (10 mmol, 1.0 eq) in methanol (100 ml) at 0° C. was added thionyl chloride (18.38 ml, 251.84 mmol, 3.0 eq). The reaction mixture was heated to reflux for about 3 hours. TLC indicated starting material was completed and the desired product was observed. The reaction mixture was evaporated under reduced pressure, ethyl acetate (100 ml) and triethylamine (35.39 ml, 251.84 mmol, 3.0 eq) were added to the reaction mixture, precipitate was formed. The precipitates formed were collected by filtration and the filtrate was evaporated under reduced pressure to obtain the title compound (8 g) as oil. The obtained compound was used as such for next step without further purification. (MWt. 13307, Observed MWt 134.05).

### Step 2: Synthesis of methyl 3-(1,1-dioxidothiomorpholino)-4-hydroxybutanoate

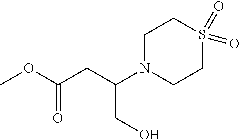 [SMILES: COC(=O)CC(CO)N1CCS(=O)(=O)CC1]

To a stirred solution of methyl 3-amino-4-hydroxybutanoic acid (step 1, 2.5 g, 1.0 eq) in ethanol (25 ml) and 1,4-dioxane (25 ml) was added triethylamine (7.91 ml, 56.31 mmol, 3.0 eq) and divinyl sulfone (5.61 ml, 37.55 mmol, 2.0 eq). The reaction mixture was heated to reflux for about 3 hours. TLC indicated starting material was consumed and the desired product was observed. The reaction mixture was evaporated under reduced pressure. The residue was purified by silica gel column chromatography using 2% methanol in DCM as an eluent to obtain the title compound (4 g, 80% yield) as a solid. (MWt: 251.08; Observed MWt 252.10).

### Step 3: Synthesis of methyl 4-((tert-butyl dimethylsilyl)oxy)-3-(1,1-dioxidothiomorpholino)butanoate

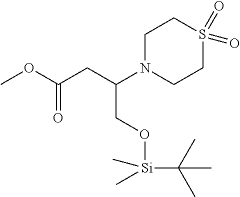 [SMILES: COC(=O)CC(CO[Si](C)(C)C(C)(C)C)N1CCS(=O)(=O)CC1]

To a stirred solution of methyl 3-(1,1-dioxidothiomorpholino)-4-hydroxybutanoate (step 2, 1.5 g, 1.0 eq) in DCM (15 ml) and DMF (10 ml) was added imidazole (0.813 g, 11.952 mmol, 2.0 eq) and tert-Butyldimethylsilyl chloride (1.35 g, 8.964 mmol, 1.5 eq). The reaction mixture was stirred at room temperature for overnight. TLC indicated starting material was consumed and the desired product was observed. The reaction mixture was diluted with water (20 ml) and extracted with DCM (2×30 ml). The combined organic layer was separated, dried over Na₂SO₄, filtered and concentrated under reduced pressure. The residue was purified by silica gel column chromatography using 1% methanol in DCM as an eluent to obtain the title compound (0.6 g, 27.52% yield) as an off-white solid. (M.Wt: 365.17 and observed MWt: 366. 10)

### Step 4: Synthesis of 4-(1-((tert-butyldimethylsilyl)oxy)-4-hydroxybutan-2-yl)thiomorpholine 1,1-dioxide

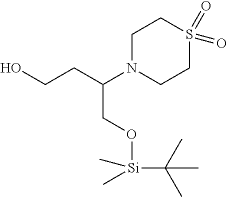 [SMILES: CC(C)(C)[Si](C)(C)OCC(CCO)N1CCS(=O)(=O)CC1]

To a stirred solution of methyl methyl 4-((tert-butyl dimethylsilyl)oxy)-3-(1,1-dioxidothiomorpholino)butanoate (step 3, 0.6 g, 1.0 eq) in DCM (12 ml) at −78° C. was added DIBAL-H (6.5 ml, 9.847 mmol, 6.0 eq, 1.5M in toluene). The reaction mixture was stirred at same temperature for about 4 hours. TLC indicated starting material was consumed and the desired product was observed. The reaction mixture was quenched with saturated ammonium chloride solution and extracted with ethyl acetate (2×30 ml). The organic layer was washed with water, dried over Na₂SO₄, filtered and concentrated under reduced pressure. The residue was purified by silica gel column chromatography using 2% methanol in DCM as an eluent to obtain the title compound (0.442 g, 75% yield) as an off-white solid. (M.Wt: 337.17; Observed M/Wt: 338.15)

### EXAMPLES

### Example 1: Preparation of (1R,3S)-3-((((1R,3aS,5aR,5bR,7aR,9S,11aR,11bR,13aR,13bR)-3a-(((R)-2-(1,1-dioxidothiomorpholino)-3-methoxy-3-oxopropyl)amino)-5a,5b,8,8,11a-penta methyl-1-(1-methylcyclopropyl)icosahydro-1H-cyclopenta[a]chrysen-9-yl)oxy)carbonyl)-2,2-dimethylcyclobutane-1-carboxylic acid

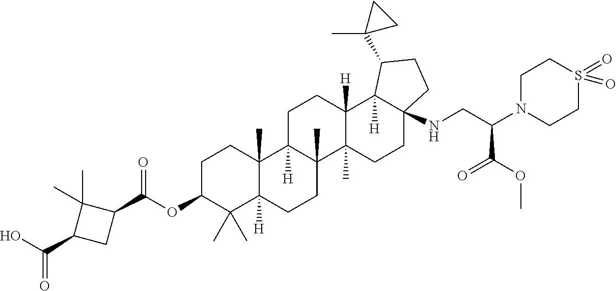

### Step 1: Synthesis of 1-benzyl 3-((1R,3aS,5aR,5bR,7aR,9S,11aR,11bR,13aR,13bR)-3a-(((R)-2-(1,1-dioxidothiomorpholino)-3-methoxy-3-oxopropyl)amino)-5a,5b,8,8,11a-pentamethyl-1-(1-methylcyclopropyl)icosahydro-1H-cyclopenta[a]chrysen-9-yl) (1R,3S)-2,2-dimethylcyclobutane-1,3-dicarboxylate

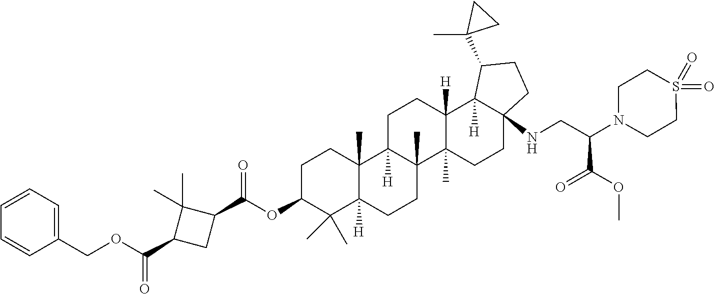

To a stirred solution of methyl (R)-2-(1, 1-dioxidothiomorpholino)-3-hydroxypropanoate (Intermediate 2, 0.34 g, 1.457 mmol, 1.25 eq) in dichloromethane (16 ml) at 0° C. was added trifluoromethanesulfonic anhydride (0.27 ml, 1.632 mmol, 1.4 eq). The reaction mixture was stirred at 0° C. for 10 minutes, then 2,6-lutidine (0.19 ml, 1.667 mmol, 1.43 eq) was added and stirred at same temperature for 10 minutes. 1-((1R,3aS,5aR,5bR,7aR, 9S,11aR,11bR,13aR,13bR)-3a-amino-5a,5b,8,8,11a-pentamethyl-1-(1-methylcyclopropyl) icosahydro-1H-cyclopenta[a]chrysen-9-yl) 3-benzyl (1S,3R)-2,2-dimethylcyclobutane-1,3-dicarboxylate (Intermediate 1, 0.8 g, 1.166 mmol, 1.0 eq) followed by triethylamine (0.23 ml, 1.667 mmol, 1.43 eq) were added to the reaction mixture and stirred at 0° C. for about 1 hour. The reaction mixture was changed to room temperature and stirred for about 4 hours. TLC indicated starting material was completed and the desired product was observed. The reaction mixture was basified with saturated sodium bicarbonate solution and extracted with dichloromethane (3×50 ml). The combined organic layer was washed with water (20 ml), dried over Na₂SO₄, filtered and concentrated under reduced pressure. The crude compound was purified by silica gel column chromatography using 0-3% methanol in dichloromethane gradient to obtain the title compound (0.680 g, 64.7% yield) as a solid. ¹H NMR (300 MHz, CDCl₃): δ ppm 7.35 (m, 5H), 5.15, 5.09 (ABq, J_{AB}=12.3 Hz, 2H), 4.44 (dd, J=11.1, 4.5 Hz 1H), 3.70 (s, 3H), 3.42-3.35 (m, 1H), 3.35-3.25 (m, 1H), 3.15-2.94 (m, 7H), 2.85-2.57 (m, 5H), 2.08-1.0 (m, 28H), 1.0-0.78 (m, 22H), 0.38-0.20 (m, 4H).

### Step 2: Synthesis of (1R,3S)-3-((((1R,3aS,5aR,5bR,7aR,9S,11aR,11bR,13aR,13bR)-3a-(((R)-2-(1,1-dioxidothiomorpholino)-3-methoxy-3-oxopropyl)amino)-5a,5b,8,8,11a-pentamethyl-1-(1-methylcyclopropyl)icosahydro-1H-cyclopenta[a]chrysen-9-yl)oxy)carbonyl)-2,2-dimethyl cyclobutane-1-carboxylic acid

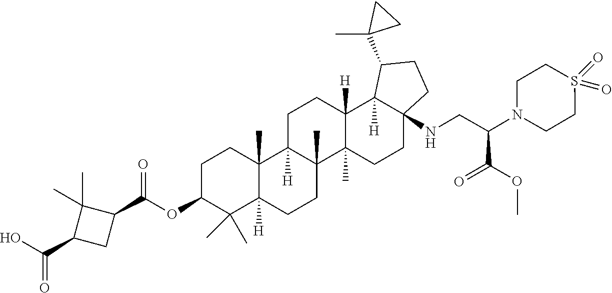

To a suspension of 10% Pd/C (500 mg) in ethyl acetate (7 ml) and methanol (7 ml) was added 1-benzyl 3-((1R,3aS,5aR,5bR,7aR,9S,11aR,11bR,13aR,13bR)-3a-(((R)-2-(1,1-dioxidothiomorpholino)-3-methoxy-3-oxopropyl)amino)-5a,5b,8,8,11a-pentamethyl-1-(1-methylcyclopropyl)icosahydro-1H-cyclopenta[a]chrysen-9-yl) (1R,3S)-2,2-dimethylcyclo butane-1,3-dicarboxylate (step 1, 0.35 g, 0.386 mmol, 1.0 eq) and ammonium formate (0.121 g, 1.933 mmol, 5.0 eq). The reaction mixture was stirred at room temperature for about 1 hour. TLC indicated starting material was completed and the desired product was observed. The reaction mixture was filtered through celite pad and washed with 5% methanol in DCM (100 ml). The filtrate was evaporated under reduced pressure and the residue was purified by silica gel column chromatography using 5 to 10% methanol in DCM with one drop acetic acid gradient. The obtained solid was treated with ethyl acetate (2 ml) and hexane (8 mL) and stirred at room temperature for 20 minutes. The precipitates formed were collected by filtration and dried under vacuum to obtain the title compound (40 mg, 12.69% yield) as an off-white solid. ¹H NMR (300 MHz, CDCl₃): δ ppm 4.47-4.45 (m, 1H), 3.70 (s, 3H), 3.42-3.35 (m, 1H), 3.28 (m, 1H), 3.15-3.02 (m, 4H), 2.98 (m, 3H), 2.85-2.75 (m, 2H), 2.65-2.52 (m, 3H), 2.10-1.18 (m, 25H), 1.10-0.78 (m, 25H), 0.40-0.20 (m, 4H); ES-MS: m/z 815.88 (M+H)⁺.

### Example 2: Preparation of 5-(((1R,3aS,5aR,5bR,7aR,9S,11aR,11bR,13aR,13bR)-3a-(((2R,3R)-2-(1,1-dioxidothiomorpholino)-3-hydroxybutyl)amino)-5a,5b,8,8,11a-pentamethyl-1-(prop-1-en-2-yl)icosahydro-1H-cyclopenta[a]chrysen-9-yl)oxy)-3,3-dimethyl-5-oxopentanoic acid

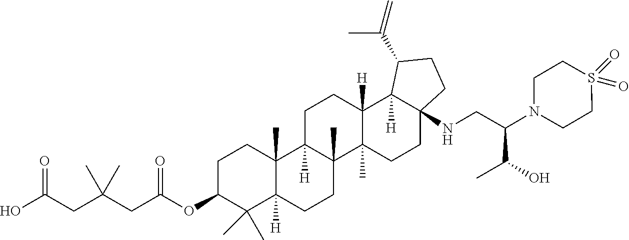

### Step 1: Synthesis of (1R,3aS,5aR,5bR,7aR,9S,11aR,11bR,13aR,13bR)-3a-((tert-butoxycarbonyl)amino)-5a,5b,8,8,11a-pentamethyl-1-(prop-1-en-2-yl)icosahydro-1H-cyclopenta[a]chrysen-9-yl acetate

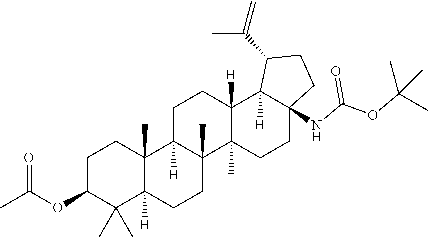

To a stirred solution of tert-butyl ((1R,3aS,5aR,5bR,7aR,9S,11aR,11bR,13aR,13bR)-9-hydroxy-5a,5b,8,8,11a-pentamethyl-1-(prop-1-en-2-yl)icosahydro-3aH-cyclopenta[a]chrysen-3a-yl)carbamate (Prepared as described in WO 2017/149518 A2, 12 g, 22.734 mmol, 1.0 eq) in THF (120 ml) at 0° C. was added triethylamine (9.5 ml, 68.203 mmol, 3.0 eq), DMAP (0.333 g, 2.728 mmol, 0.12 eq) and acetic anhydride (3 ml, 31.828 mmol, 1.4 eq). The reaction mixture was stirred at room temperature for overnight. TLC indicated starting material was consumed and the desired product was observed. The reaction mixture was diluted with ethyl acetate (200 ml) and washed with water (2×200 ml). The organic layer was dried over sodium sulfate, filtered and concentrated under reduced pressure. The crude compound was treated with n-hexane (100 ml), stirred at room temperature for about 1 hour, filtered and dried under vacuum to obtain the title compound (12 g, 93% yield) as an off-white solid. ¹H NMR (300 MHz, CDCl₃): δ ppm 4.71 (s, 1H), 4.60 (s, 1H), 4.47 (dd, J=9.9, 4.5 Hz, 1H), 4.34 (s, 1H), 2.58-2.30 (m, 3H), 2.05 (s, 3H), 2.02-0.77 (m, 22H), 1.68 (s, 3H), 1.44 (s, 9H), 1.01 (s, 3H), 0.95 (s, 3H), 0.85 (s, 3H), 0.84 (s, 3H), 0.83 (s, 3H); ES-MS: m/z 570.39 (M+H)⁺.

### Step 2: Synthesis of (1R,3aS,5aR,5bR,7aR,9S,11aR,11bR,13aR,13bR)-3a-amino-5a,5b,8,8,11a-pentamethyl-1-(prop-1-en-2-yl)icosahydro-1H-cyclopenta[a]chrysen-9-yl acetate

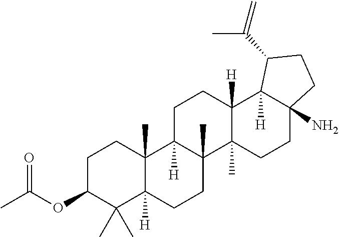

To a stirred solution of (1R,3aS,5aR,5bR,7aR,9S,11aR,11bR,13aR,13bR)-3a-((tert-butoxycarbonyl)amino)-5a,5b,8,8,11a-pentamethyl-1-(prop-1-en-2-yl)icosahydro-1H-cyclopenta[a]chrysen-9-yl acetate (step 1, 12 g, 21.057 mmol, 1.0 eq) in 1,4-dioxane (24 ml) was added 4N HCl in 1,4-dioxane (24 ml). The reaction mixture was stirred at room temperature for overnight. TLC indicated starting material was consumed and the desired product was observed. The reaction mixture was concentrated under reduced pressure and basified with saturated sodium bicarbonate solution. The obtained solid was filtered and washed with water. The solid was dissolved in DCM, dried over sodium sulfate, filtered and concentrated under reduced pressure. The solid was further purified by trituration with n-Hexane (100 ml), filtered, washed with hexane and dried under vacuum to obtain the title compound (7 g, 70.7% yield) as an off-white solid. ¹H NMR (300 MHz, CDCl₃): δ ppm 4.71 (s, 1H), 4.59 (s, 1H), 4.47 (dd, J=9.0, 5.4 Hz, 1H), 2.57-2.48 (m, 1H), 2.04 (s, 3H), 1.68 (s, 3H), 1.66-0.77 (m, 24H), 1.04 (s, 3H), 0.95 (s, 3H), 0.85 (s, 3H), 0.84 (s, 3H), 0.83 (s, 3H); ES-MS: m/z 470.44 (M+H)⁺.

### Step 3: Synthesis of (1R,3aS,5aR,5bR,7aR,9S,11aR,11bR,13aR,13bR)-3a-(((2R,3R)-3-((tert-butyldimethylsilyl)oxy)-2-(1,1-dioxidothiomorpholino)butyl)amino)-5a,5b,8,8,11a-pentamethyl-1-(prop-1-en-2-yl)icosahydro-H-cyclopenta[a]chrysen-9-ylacetate

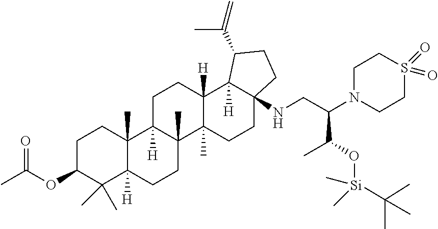

To a stirred solution of 4-((2R,3R)-3-((tert-butyldimethylsilyl)oxy)-1-hydroxybutan-2-yl)thiomorpholine 1,1-dioxide (Intermediate-3, 8.5 g, 25.326 mmol, 1.7 eq) in DCM (70 ml) at 0° C. was added 2,6-Lutidine (3.8 ml, 32.775 mmol, 2.2 eq). After 10 minutes stirring at 0° C., trifluoromethane sulfonic anhydride (5 ml, 29.795 mmol, 2.0 eq) was added and stirred at 0° C. for about 20 minutes. (1R,3aS,5aR,5bR,7aR,9S,11aR,11bR,13aR,13bR)-3a-amino-5a,5b,8,8,11a-pentamethyl-1-(prop-1-en-2-yl)icosahydro-1H-cyclopenta[a]chrysen-9-yl acetate (step 2, 7 g, 14.897 mmol, 1.0 eq) and triethylamine (7.27 ml, 52.142 mmol, 3.5 eq) were added to the reaction mixture and stirred at 0° C. for about 1 hour. The reaction mixture was stirred at room temperature for overnight. TLC indicated starting material was consumed and the desired product was observed. The reaction mixture was diluted with DCM (200 ml), washed with saturated sodium carbonate solution (100 ml) and water (100 ml). The organic layer was dried over sodium sulfate, filtered and concentrated under reduced pressure. The crude product was purified by silica gel column chromatography using 0-10% ethyl acetate in hexane gradient. The obtained compound was further purified by treating with n-hexane, filtered and dried under vacuum to obtain the title compound (7.0 g, 59% yield) as an off-white solid. ¹H NMR (300 MHz, CDCl₃): δ ppm 4.68 (s, 1H), 4.58 (s, 1H), 4.49-4.44 (m, 1H), 4.08-4.0 (m, 1H), 3.44-3.39 (m, 2H), 3.28-3.22 (m, 2H), 3.06-3.01 (m, 4H), 2.60-2.40 (m, 4H), 2.04 (s, 3H), 1.93-0.76 (m, 27H), 1.68 (s, 3H), 1.04 (s, 3H), 0.94 (s, 3H), 0.87 (s, 9H), 0.85 (s, 3H), 0.84 (s, 3H), 0.83 (s, 3H), 0.07 (s, 6H); ES-MS: m/z 789.67 (M+H)⁺

### Step 4: Synthesis of 4-((2R,3R)-3-((tert-butyldimethylsilyl)oxy)-1-(((1R,3aS,5aR,55bR,7aR,9S,11aR,11bR,13aR,13bR)-9-hydroxy-5a,5b,8,8,11a-pentamethyl-1-(prop-1-en-2-yl)icosahydro-3aH-cyclopenta[a]chrysen-3a-yl)amino)butan-2-yl)thiomorpholine 1,1-dioxide

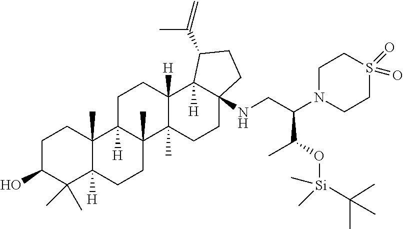

To a stirred solution of (1R,3aS,5aR,5bR,7aR,9S,11aR,11bR,13aR,13bR)-3a-(((2R,3R)-3-((tert-butyldimethylsilyl)oxy)-2-(1,1-dioxidothiomorpholino)butyl)amino)-5a,5b,8,8,11a-pentamethyl-1-(prop-1-en-2-yl)icosahydro-TH-cyclopenta[a]chrysen-9-yl acetate (step 3, 7.0 g, 8.868 mmol, 1.0 eq) in methanol (70 ml) and THF (70 ml) was added aqueous TN KOH solution (53.2 ml, 53.212 mmol, 6.0 eq). The reaction mixture was stirred at room temperature for overnight. TLC indicated starting material was consumed and the desired product was observed. The reaction mixture was concentrated under reduced pressure, DCM (400 ml) was added and washed with water (200 ml). The organic layer was dried over sodium sulfate, filtered and concentrated under reduced pressure. The crude product was purified by silica gel column chromatography using 0-30% ethyl acetate in hexanes gradient to obtain the title compound (6.0 g, 91% yield) as an off-white solid. ¹H NMR (300 MHz, CDCl₃): δ ppm 4.69 (s, 1H), 4.58 (s, 1H), 4.04-3.99 (m, 1H), 3.44-3.39 (m, 2H), 3.28-3.16 (m, 3H), 3.07-2.96 (m, 4H), 2.61-2.40 (m, 4H), 1.97-0.66 (m, 27H), 1.68 (s, 3H), 1.04 (s, 3H), 0.96 (s, 3H), 0.95 (s, 3H), 0.87 (s, 9H), 0.82 (s, 3H), 0.75 (s, 3H), 0.07 (s, 6H); ES-MS: m/z 747.74 (M+H)+

### Step 5: Synthesis of 1-((1R,3aS,5aR,5bR,7aR,9S,11 aR, 11bR,13aR,13bR)-3a-(((2R,3R)-3-((tert-butyldimethylsilyl)oxy)-2-(1,1-dioxidothiomorpholino)butyl)amino)-5a,5b,8,8,11a-pentamethyl-1-(prop-1-en-2-yl)icosahydro-1H-cyclopenta[a]chrysen-9-yl) 5-ethyl 3,3-dimethylpentanedioate

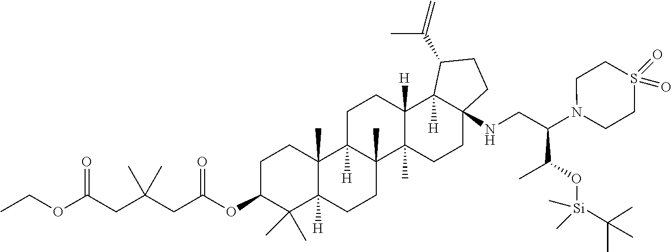

To a stirred solution of 4-((2R,3R)-3-((tert-butyldimethylsilyl)oxy)-1-(((1R,3aS,5aR,5bR,7aR,9S,11aR,11bR,13aR,13bR)-9-hydroxy-5a,5b,8,8,11a-pentamethyl-1-(prop-1-en-2-yl)icosahydro-3aH-cyclopenta[a]chrysen-3a-yl)amino)butan-2-yl)thiomorpholine 1,1-dioxide (step 4, 1.5 g, 2.007 mmol, 1.0 eq) in DCM (40 ml) at 0° C. was added 5-ethoxy-3,3-dimethyl-5-oxopentanoic acid (Prepared as described in WO 2012/143703, 0.566 g, 3.011 mmol, 1.5 eq), EDC.HCl (0.961 g, 5.018 mmol, 2.5 eq) and DMAP (0.073 g, 0.602 mmol, 0.3 eq). The reaction mixture was stirred at room temperature for overnight. TLC indicated starting material was consumed and the desired product was observed. Water (250 ml) was added to the reaction mixture, organic layer was separated and aqueous layer was extracted with DCM (3×250 ml). The combined organic layer was washed with water (100 ml), brine solution (150 ml), dried over sodium sulfate, filtered and concentrated under reduced pressure. The product was purified by silica gel column chromatography using 0-2% methanol in dichloromethane gradient. The fractions containing the expected product were combined and concentrated under reduced pressure to obtain the title compound (1.5 g, 81.45% yield) as an off-white solid. ¹H NMR (300 MHz, CDCl₃): δ ppm 4.68 (s, 1H), 4.58 (s, 1H), 4.47 (dd, J=11.4, 5.1 Hz, 1H), 4.12 (q, J=7.2 Hz, 2H), 4.05-3.97 (m, 1H), 3.46-3.37 (m, 2H), 3.31-3.21 (m, 2H), 3.05-2.97 (m, 4H), 2.62-2.51 (m, 1H), 2.50-2.34 (m, 7H), 1.97-0.75 (m, 30H), 1.68 (s, 3H), 1.11 (s, 6H), 1.04 (s, 3H), 0.95 (s, 3H), 0.87 (s, 9H), 0.85 (s, 6H), 0.82 (s, 3H), 0.07 (s, 6H); ES-MS: m/z 917.88 (M+H)⁺.

### Step 6: Synthesis of 1-((1R,3aS,5aR,5bR,7aR,9S,11aR,11bR,13aR,13bR)-3a-(((2R,3R)-2-(1,1-dioxidothiomorpholino)-3-hydroxybutyl)amino)-5a,5b,8,8,11a-pentamethyl-1-(prop-1-en-2-yl)icosahydro-1H-cyclopenta[a]chrysen-9-yl) 5-ethyl 3, 3-dimethylpentanedioate

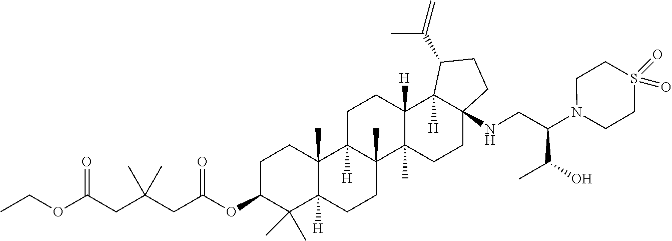

To a stirred solution of 1-((1R,3aS,5aR,5bR,7aR,9S,11aR,11bR,13aR,13bR)-3a-(((2R,3R)-3-((tert-butyldimethylsilyl)oxy)-2-(1,1-dioxidothiomorpholino)butyl)amino)-5a,5b,8,8,11a-pentamethyl-1-(prop-1-en-2-yl)icosahydro-1H-cyclopenta[a]chrysen-9-yl) 5-ethyl 3,3-dimethylpentanedioate (step 5, 1.5 g, 1.634 mmol, 1.0 eq) in THF (15 ml) at 0° C. was added TBAF (6.54 ml, 6.54 mmol, 4.0 eq, 1.0M in THF). The reaction mixture was stirred at room temperature for overnight. TLC indicated starting material was consumed and the desired product was observed. Water (200 ml) was added to the reaction mixture and extracted with DCM (3×250 ml). The combined organic layer was dried over sodium sulfate, filtered and concentrated under reduced pressure. The product was purified by silica gel column chromatography using 04% methanol in dichloromethane gradient. The fractions containing the expected product were combined and concentrated under reduced pressure to obtain the title compound (1.0 g, 76.92% yield) as an off-white solid. ¹H NMR (300 MHz, CDCl₃): δ ppm 4.69 (s, 1H), 4.59 (s, 1H), 4.47 (dd, J=11.7, 5.1 Hz, 1H), 4.12 (q, J=7.2 Hz, 2H), 3.73-3.68 (m, 1H), 3.43-3.38 (m, 2H), 3.18-3.05 (m, 6H), 2.65-2.40 (m, 8H), 1.93-0.76 (m, 30H), 1.68 (s, 3H), 1.11 (s, 6H), 1.04 (s, 3H), 0.95 (s, 3H), 0.85 (s, 6H), 0.83 (s, 3H); ES-MS: m/z 803.81 (M+H)⁺.

### Step 7: Synthesis of 5-(((1R,3aS,5aR,5bR,7aR,9S,11 aR, 11bR,13aR,13bR)-3a-(((2R,3R)-2-(1,1-dioxidothiomorpholino)-3-hydroxybutyl)amino)-5a,5b,8,8,11a-pentamethyl-1-(prop-1-en-2-yl)icosahydro-1H-cyclopenta[a]chrysen-9-yl)oxy)-3, 3-dimethyl-5-oxopentanoic acid

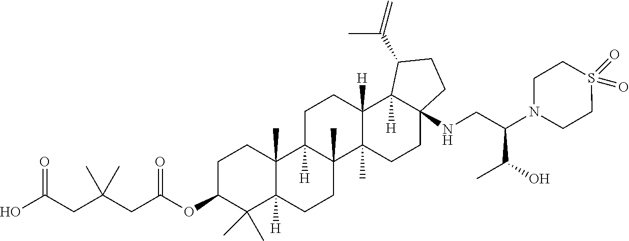

To a stirred solution of 1-((1R,3aS,5aR,5bR,7aR,9S,11aR,11bR,13aR,13bR)-3a-(((2R,3R)-2-(1,1-dioxidothiomorpholino)-3-hydroxybutyl)amino)-5a,5b,8,8,11a-pentamethyl-1-(prop-1-en-2-yl)icosahydro-1H-cyclopenta[a]chrysen-9-yl) 5-ethyl 3,3-dimethylpentanedioate (step 6, 1.0 g, 1.245 mmol, 1.0 eq) in methanol (10 ml) and THF (10 ml) at 0° C. was added aqueous 2.5N KOH solution (3.73 ml, 9.337 mmol, 7.5 eq). The reaction mixture was stirred at room temperature for overnight. TLC indicated starting material was consumed and the desired product was observed. The reaction mixture was evaporated under reduced pressure, cooled to 0° C., water (10 ml) was added, acidified to pH 3.0 with 1N HCl and extracted with DCM (3×200 ml). The combined organic layer was washed with water (150 ml), dried over sodium sulfate, filtered and concentrated under reduced pressure. The product was purified by silica gel column chromatography using 0-3% methanol in dichloromethane gradient. The fractions containing the expected product were combined and concentrated under reduced pressure to obtain the title compound (0.680 g, 70.46% yield) as an off-white solid. ¹H NMR (300 MHz, CDCl₃): δ ppm 4.69 (s, 1H), 4.59 (s, 1H), 4.55-4.47 (m, 1H), 3.75-3.69 (m, 1H), 3.43-3.36 (m, 2H), 3.19-3.04 (m, 6H), 2.65-2.58 (m, 1H), 2.54-2.37 (m, 7H), 1.97-0.77 (m, 27H), 1.68 (s, 3H), 1.14 (s, 6H), 1.05 (s, 3H), 0.96 (s, 3H), 0.86 (s, 6H), 0.84 (s, 3H); ES-MS: m/z 776.04 (M+H)⁺.

### Example 3: Preparation of 5-(((1R,3aS,5aR,5bR,7aR,9S,11aR,11bR,13aR,13bR)-3a-((3-(1,1-dioxidothiomorpholino)-4-hydroxybutyl)amino)-5a,5b,8,8,11a-pentamethyl-1-(prop-1-en-2-yl)icosahydro-1H-cyclopenta[a]chrysen-9-yl)oxy)-3,3-dimethyl-5-oxopentanoic acid

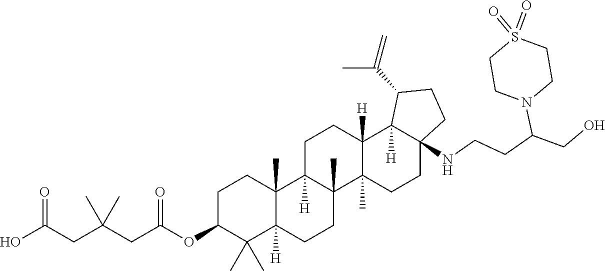

### Step 1: Synthesis of (1R,3aS,5aR,5bR,7aR,9S,11aR,11bR,13aR,13bR)-3a-((4-((tert-butyldimethylsilyl)oxy)-3-(1,1-dioxidothiomorpholino)butyl)amino)-5a,5b,8,8,11a-pentamethyl-1-(prop-1-en-2-yl)icosahydro-1H-cyclopenta[a]chrysen-9-yl acetate

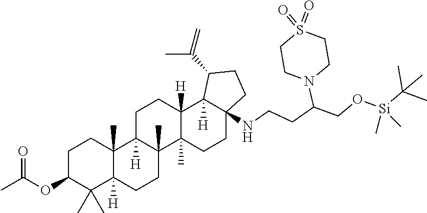

To a stirred solution of 4 4-(1-((tert-butyldimethylsilyl)oxy)-4-hydroxybutan-2-yl)thiomorpholine 1,1-dioxide (Intermediate-5, 8.5 g, 1.7 eq) in DCM (70 ml) at 0° C. was added 2,6-Lutidine (3.8 ml, 32.775 mmol, 2.2 eq). After 10 minutes stirring at 0° C., trifluoromethane sulfonic anhydride (5 ml, 29.795 mmol, 2.0 eq) was added and stirred at 0° C. for about 20 minutes. (1R,3aS,5aR,5bR,7aR,9S,11aR,11bR,13aR,13bR)-3a-amino-5a,5b,8,8,11a-pentamethyl-1-(prop-1-en-2-yl)icosahydro-1H-cyclopenta[a]chrysen-9-yl acetate (Example 2-step 2, 7 g, 14.897 mmol, 1.0 eq) and triethylamine (7.27 ml, 52.142 mmol, 3.5 eq) were added to the reaction mixture and stirred at 0° C. for about 1 hour. The reaction mixture was stirred at room temperature for overnight. TLC indicated starting material was consumed and the desired product was observed. The reaction mixture was diluted with DCM (200 ml), washed with saturated sodium carbonate solution (100 ml) and water (100 ml). The organic layer was dried over sodium sulfate, filtered and concentrated under reduced pressure. The crude product was purified by silica gel column chromatography using 0-10% ethyl acetate in hexane gradient. The obtained compound was further purified by treating with n-hexane, filtered and dried under vacuum to obtain the title compound (7.0 g, 60% yield) as an off-white solid. ES-MS: m/z 789.29 (M+H)⁺.

### Step 2: Synthesis of 4-(1-((tert-butyldimethylsilyl)oxy)-4-(((1R,3aS,5aR,5bR,7aR,9S,11aR,11bR,13aR,13bR)-9-hydroxy-5a,5b,8,8,11a-pentamethyl-1-(prop-1-en-2-yl)icosahydro-3aH-cyclopenta[a]chrysen-3a-yl)amino)butan-2-yl)thiomorpholine 1,1-dioxide

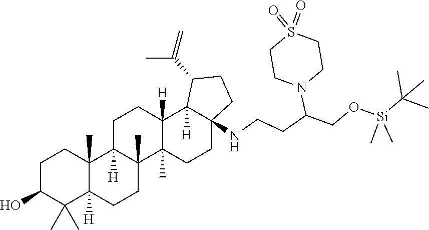

To a stirred solution of (1R,3aS,5aR,5bR,7aR,9S,11aR,11bR,13aR,13bR)-3a-((4-((tert-butyldimethylsilyl)oxy)-3-(1,1-dioxidothiomorpholino)butyl)amino)-5a,5b,8,8,11a-pentamethyl-1-(prop-1-en-2-yl)icosahydro-1H-cyclopenta[a]chrysen-9-yl acetate (step 1, 7.0 g, 1.0 eq) in methanol (70 ml) and THF (70 ml) was added aqueous 1N KOH solution (53.2 ml, 53.212 mmol, 6.0 eq). The reaction mixture was stirred at room temperature for overnight. TLC indicated starting material was consumed and the desired product was observed. The reaction mixture was concentrated under reduced pressure, DCM (400 ml) was added and washed with water (200 ml). The organic layer was dried over sodium sulfate, filtered and concentrated under reduced pressure. The crude product was purified by silica gel column chromatography using 0-30% ethyl acetate in hexanes gradient to obtain the title compound (6.0 g, 91% yield) as an off-white solid.; ES-MS: m/z 747.74 (M+H)⁺.

### Step 3: Synthesis of 1-((1R,3aS,5aR,5bR,7aR,9S,11aR,11bR,13aR,13bR)-3a-((4-((tert-butyldimethylsilyl)oxy)-3-(1,1-dioxidothiomorpholino)butyl)amino)-5a,5b,8,8,11a-pentamethyl-1-(prop-1-en-2-yl)icosahydro-1H-cyclopenta[a]chrysen-9-yl) 5-ethyl 3,3-dimethylpentanedioate

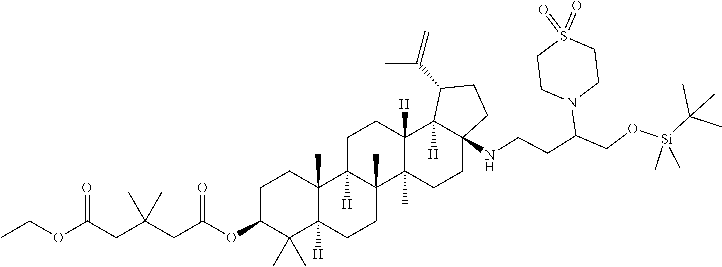

To a stirred solution of 4-(1-((tert-butyldimethylsilyl)oxy)-4-(((1R,3aS,5aR,5bR,7aR,9S,11aR,11bR,13aR,13bR)-9-hydroxy-5a,5b,8,8,11a-pentamethyl-1-(prop-1-en-2-yl)icosahydro-3aH-cyclopenta[a]chrysen-3a-yl)amino)butan-2-yl)thiomorpholine 1,1-dioxide (step 2, 1.5 g, 1.0 eq) in DCM (40 ml) at 0° C. was added 5-ethoxy-3,3-dimethyl-5-oxopentanoic acid (Prepared as described in WO 2012/143703, 0.566 g, 3.011 mmol, 1.5 eq), EDC.HCl (0.961 g, 5.018 mmol, 2.5 eq) and DMAP (0.073 g, 0.602 mmol, 0.3 eq). The reaction mixture was stirred at room temperature for overnight. TLC indicated starting material was consumed and the desired product was observed. Water (250 ml) was added to the reaction mixture, organic layer was separated and aqueous layer was extracted with DCM (3×250 ml). The combined organic layer was washed with water (100 ml), brine solution (150 ml), dried over sodium sulfate, filtered and concentrated under reduced pressure. The product was purified by silica gel column chromatography using 0-2% methanol in dichloromethane gradient. The fractions containing the expected product were combined and concentrated under reduced pressure to obtain the title compound (1.5 g, 81.45% yield) as an off-white solid.; ES-MS: m/z 917.88 (M+H)⁺.

### Step 4: Synthesis of 1-((1R,3aS,5aR,5bR,7aR,9S,11aR,11bR,13aR,13bR)-3a-((3-(1,1-dioxidothiomorpholino)-4-hydroxybutyl)amino)-5a,5b,8,8,11a-pentamethyl-1-(prop-1-en-2-yl)icosahydro-1H-cyclopenta[a]chrysen-9-yl) 5-ethyl 3,3-dimethylpentanedioate

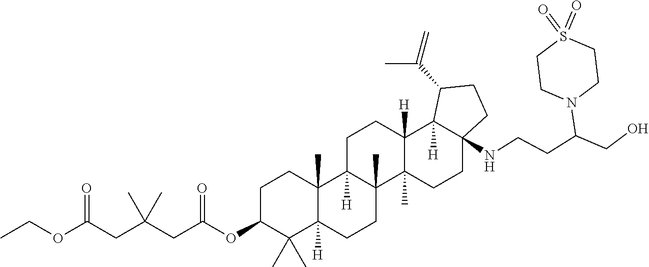

To a stirred solution of 1-((1R,3aS,5aR,5bR,7aR,9S,11aR,11bR,13aR,13bR)-3a-((4-((tert-butyldimethylsilyl)oxy)-3-(1,1-dioxidothiomorpholino)butyl)amino)-5a,5b,8,8,11a-pentamethyl-1-(prop-1-en-2-yl)icosahydro-1H-cyclopenta[a]chrysen-9-yl) 5-ethyl 3,3-dimethylpentanedioate (step 3, 1.5 g, 1.634 mmol, 1.0 eq) in THF (15 ml) at 0° C. was added TBAF (6.54 ml, 6.54 mmol, 4.0 eq, 1.0M in THF). The reaction mixture was stirred at room temperature for overnight. TLC indicated starting material was consumed and the desired product was observed. Water (200 ml) was added to the reaction mixture and extracted with DCM (3×250 ml). The combined organic layer was dried over sodium sulfate, filtered and concentrated under reduced pressure. The product was purified by silica gel column chromatography using 04% methanol in dichloromethane gradient. The fractions containing the expected product were combined and concentrated under reduced pressure to obtain the title compound (1.0 g, 76.92% yield) as an off-white solid. ES-MS: m/z 803.81 (M+H)⁺.

### Step 5: Synthesis of 5-(((1R,3aS,5aR,5bR,7aR,9S,11aR,11bR,13aR,13bR)-3a-((3-(1,1-dioxidothiomorpholino)-4-hydroxybutyl)amino)-5a,5b,8,8,11a-pentamethyl-1-(prop-1-en-2-yl)icosahydro-1H-cyclopenta[a]chrysen-9-yl)oxy)-3, 3-dimethyl-5-oxopentanoic acid

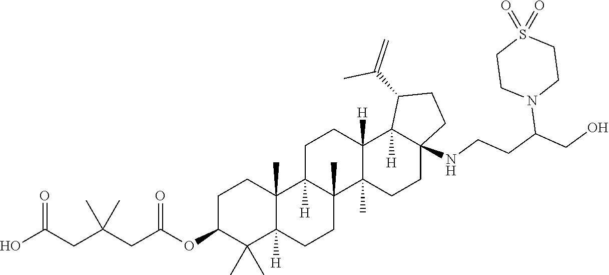

To a stirred solution of 1-((1R,3aS,5aR,5bR,7aR,9S,11aR,11bR,13aR,13bR)-3a-((3-(1,1-dioxidothiomorpholino)-4-hydroxybutyl)amino)-5a,5b,8,8,11a-pentamethyl-1-(prop-1-en-2-yl)icosahydro-1H-cyclopenta[a]chrysen-9-yl) 5-ethyl 3,3-dimethylpentanedioate (step 4, 1.0 g, 1.245 mmol, 1.0 eq) in methanol (10 ml) and THF (10 ml) at 0° C. was added aqueous 2.5N KOH solution (3.73 ml, 9.337 mmol, 7.5 eq). The reaction mixture was stirred at room temperature for overnight. TLC indicated starting material was consumed and the desired product was observed. The reaction mixture was evaporated under reduced pressure, cooled to 0° C., water (10 ml) was added, acidified to pH 3.0 with 1N HCl and extracted with DCM (3×200 ml). The combined organic layer was washed with water (150 ml), dried over sodium sulfate, filtered and concentrated under reduced pressure. The product was purified by silica gel column chromatography using 0-3% methanol in dichloromethane gradient. The fractions containing the expected product were combined and concentrated under reduced pressure to obtain the title compound (0.680 g, 70.46% yield) as an off-white solid. ES-MS: m/z 775.04 (M+H)⁺.

### BIOLOGICAL ACTIVITY

The compounds described herein are tested for their antiviral activity following procedures known to a person of ordinary skill in the art. For example, the following protocols can be employed for testing the compounds. These protocols are illustrative and do not limit to the scope of the invention.

Evaluation of Compounds Antiviral Activity Against HIV-1 Strain 92HT599 in MT2 Cells:

MT2 cells were infected with HIV-1 strain 92HT599 (10 TCID 50/30000 cells). The infected cells were plated at the concentration of ˜30,000 cells per well in 96 well plate. Test compound was added to the micro plate in defined format with the final concentration of DMSO (vehicle) is not more than 1%. Incubation was carried out in CO₂ incubator for ˜ 96 hours for viral infection. At the end of incubation period an aliquot from each well was taken for p24 estimation. The quantitation of p24 is an index for antiviral activity of the compound. Percent inhibition was calculated with reference to control values (vehicle controls). p24 estimation was carried out using Advance biosciences kit as per the procedure detailed by supplier.

For serum binding assay, the compounds of the present invention are showing IC₅₀ value of less than 50 nM.

Evaluation of Compounds Antiviral Activity Against pNL4-3/WT & V7A Strains in MT4 Cells:

MT4 cells were Transfected with HIV-1 Plasmid (pNL4-3-WT & V7A) (Cells were incubating with required number of TCID50 of HIV-1 for 1.5 h at 37° C.). After infection, the infected cells were plated at the concentration of 3×10⁴ cells per well in 96 well plate. Test compound was added to the test plate in defined format with the final concentration of DMSO is not more than 1%. Incubation was carried out in CO₂ incubator for 4 days for viral infection. At the end of incubation period an aliquot from each well was taken for p24 estimation. p24 estimation was carried out using Advance biosciences kit as per the procedure detailed by supplier.

For pNL4-3 WT assay, the compounds of the present invention shows IC₅₀ value less than 100 nM;

For pNL4-3 V7A assay, the compounds of the present invention shows IC₅₀ value less than 100 nM;

### REFERENCES

1. Antiviral methods and protocols (Eds: D Kinchington and R. F. Schinazi) Humana Press Inc., 2000.
2. HIV protocols (Eds: N. L. Michael and J. H. Kim) Humana Press Inc, 1999.
3. DAIDS Virology manual from HIV laboratories, Publication NIH-97-3838, 1997.
4. HIV-1 p24 antigen capture assay, enzyme immunoassay for detection of Human immunodeficiency Virus Type 1 (HIV-1) p24 in tissue culture media—Advanced bio science laboratories, Inc kit procedure.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as described above.

All publications and patent applications cited in this application are herein incorporated by reference to the same extent as if each individual publication or patent application was specifically and individually indicated to be incorporated herein by reference.

1. A compound of the formula (I) 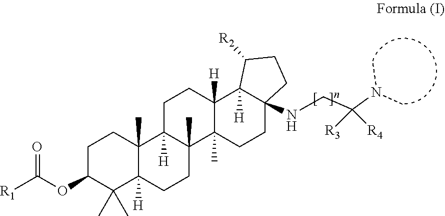
wherein,
R₁ is selected from 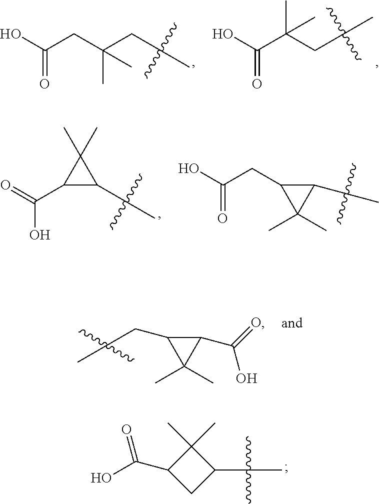
R₂ is selected from C₁-C₆ alkyl, optionally substituted C₂-C₆ alkenyl, and optionally substituted C₃-C₈ cycloalkyl; wherein the optional substituent is C₁-C₆ alkyl;
R₃ is hydrogen or C₁-C₆ alkyl;
R₄ is selected from optionally substituted C₁-C₆ alkyl, and —C(O)ORₐ; wherein the optional substituent is selected from halo, hydroxy, alkoxy, —OC(O)CH₂alkoxy, —OSi(Rₐ)₃, —N(Rₐ)(R_{b}), —S(Rₐ), and —O—CH₂—P(O)(ORₐ)₂;
ring 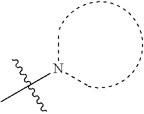
is 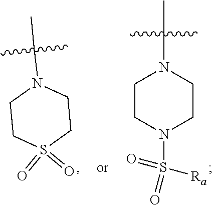
Rₐ is hydrogen or C₁-C₆ alkyl;
R_{b} is selected from hydrogen, alkyl, —C(O)alkoxy, and —S(O)₂alkyl, and
‘n’ is an integer selected from 1-2; or a
pharmaceutically acceptable salt, pharmaceutically acceptable stereoisomer, prodrug, or combination thereof.

2. The compound according to claim 1, which is a compound of the formula (IA): 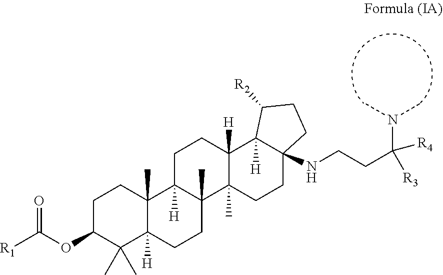
wherein,
R₁ is selected from 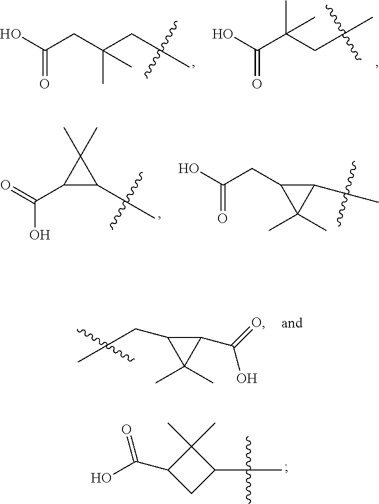
R₂ is selected from C₁-C₆ alkyl, optionally substituted C₂-C₆ alkenyl, and optionally substituted C₃-C₈ cycloalkyl; wherein the optional substituent is C₁-C₆ alkyl;
R₃ is hydrogen or C₁-C₆ alkyl;
R₄ is selected from optionally substituted C₁-C₆ alkyl, and —C(O)ORₐ; wherein the optional substituent is selected from halo, hydroxy, alkoxy, —OC(O)CH₂alkoxy, —OSi(Rₐ)₃, —N(Rₐ)(R_{b}), —S(Rₐ), and —O—CH₂—P(O)(ORₐ)₂;
ring 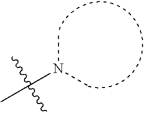
is 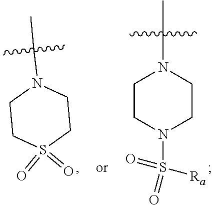
Rₐ is hydrogen or C₁-C₆ alkyl; and
R_{b} is selected from hydrogen, alkyl, —C(O)alkoxy, and —S(O)₂alkyl, or a
pharmaceutically acceptable salt, pharmaceutically acceptable stereoisomer, prodrug, or combination thereof.

3. The compound according to claim 1, which is a compound of the formula (IB) 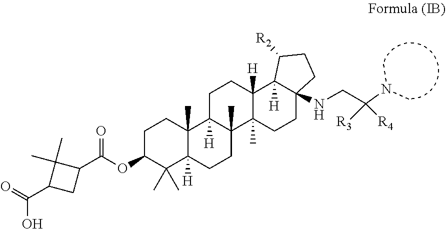
wherein,
R₂ is selected from C₁-C₆ alkyl, optionally substituted C₂-C₆ alkenyl, and optionally substituted C₃-C₈ cycloalkyl; wherein the optional substituent is C₁-C₆ alkyl;
R₃ is hydrogen or C₁-C₆ alkyl;
R₄ is selected from optionally substituted C₁-C₆ alkyl, and —C(O)ORₐ; wherein the optional substituent is selected from halo, hydroxy, alkoxy, —OC(O)CH₂alkoxy, —OSi(Rₐ)₃, —N(Rₐ)(R_{b}), —S(Rₐ), and —O—CH₂—P(O)(ORₐ)₂;
ring 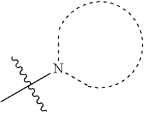
is 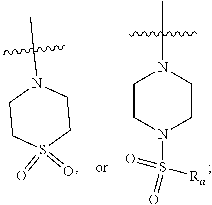
Rₐ is hydrogen or C₁-C₆ alkyl; and
R_{b} is selected from hydrogen, alkyl, —C(O)alkoxy, or —S(O)₂alkyl, or
pharmaceutically acceptable salts, pharmaceutically acceptable stereoisomers, prodrugs, or combination thereof.

4. The compound according to claim 1, which is a compound of the formula (IC) 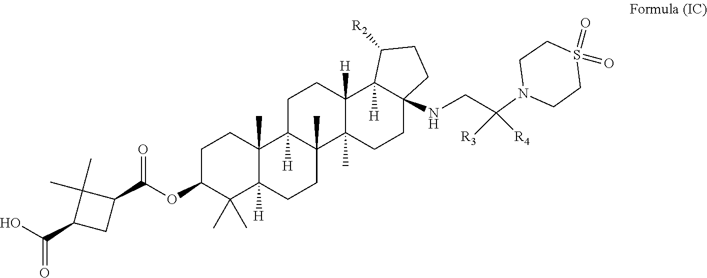
wherein,
R₂ is selected from C₁-C₆ alkyl, optionally substituted C₂-C₆ alkenyl, and optionally substituted C₃-C₈ cycloalkyl; wherein the optional substituent is C₁-C₆ alkyl;
R₃ is hydrogen or C₁-C₆ alkyl;
R₄ is selected from optionally substituted C₁-C₆ alkyl and —C(O)ORₐ; wherein the optional substituent is selected from halo, hydroxy, alkoxy, —OC(O)CH₂alkoxy, —OSi(Rₐ)₃, —N(Rₐ)(R_{b}), —S(Rₐ), and —O—CH₂—P(O)(ORₐ)₂;
Rₐ is hydrogen or C₁-C₆ alkyl; and
R_{b} is selected from hydrogen, alkyl, —C(O)alkoxy, and —S(O)₂alkyl, or a
pharmaceutically acceptable salt, pharmaceutically acceptable stereoisomer, prodrug, or combination thereof.

5. The compound according to claim 1, which is a compound of the formula (ID) 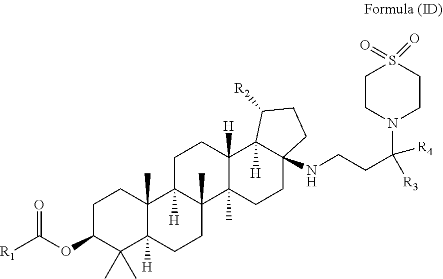
wherein,
R₁ is selected from 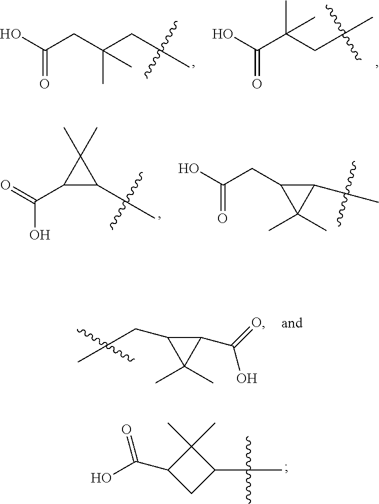
R₂ is selected from C₁-C₆ alkyl, optionally substituted C₂-C₆ alkenyl, and optionally substituted C₃-C₈ cycloalkyl; wherein the optional substituent is C₁-C₆ alkyl;
R₃ is hydrogen or C₁-C₆ alkyl;
R₄ is selected from optionally substituted C₁-C₆ alkyl and —C(O)ORₐ; wherein the optional substituent is selected from halo, hydroxy, alkoxy, —OC(O)CH₂alkoxy, —OSi(Rₐ)₃, —N(Rₐ)(R_{b}), —S(Rₐ), and —O—CH₂—P(O)(ORₐ)₂;
Rₐ is hydrogen or C₁-C₆ alkyl; and
R_{b} is selected from hydrogen, alkyl, —C(O)alkoxy, and —S(O)₂alkyl, or a
pharmaceutically acceptable salt, pharmaceutically acceptable stereoisomer, prodrug, or combination thereof.

6. A compound selected from the group consisting of:
(1R,3S)-3-((((1R,3aS,5aR,5bR,7aR,9S,11aR,11bR,13aR,13bR)-3a-(((R)-2-(1,1-dioxidothiomorpholino)-3-methoxy-3-oxopropyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
5-(((1R,3aS,5aR,5bR,7aR,9S,11aR,11bR,13aR,13bR)-3a-(((2R,3R)-2-(1,1-dioxidothiomorpholino)-3-hydroxybutyl)amino)-5a,5b,8,8,11a-pentamethyl-1-(prop-1-en-2-yl)icosahydro-1H-cyclopenta[a]chrysen-9-yl)oxy)-3,3-dimethyl-5-oxopentanoic acid,
(1R,3S)-3-((((1R,3aS,5aR,5bR,7aR,9S,11aR,11bR,13aR,13bR)-3a-(((2S,3S)-2-(1,1-dioxidothiomorpholino)-3-hydroxybutyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((2R,3S)-2-(1,1-dioxidothiomorpholino)-3-hydroxybutyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((2S,3R)-2-(1,1-dioxidothiomorpholino)-3-hydroxybutyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((2R,3S)-2-(1,1-dioxidothiomorpholino)-3-hydroxybutyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((2S,3R)-2-(1,1-dioxidothiomorpholino)-3-hydroxybutyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1R,3S)-3-((((1S,3aS,5aR,5bR,7aR,9S,11aR,11bR,13aR,13bR)-3a-(((2R,3R)-2-(1,1-dioxidothiomorpholino)-3-hydroxybutyl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1R,3S)-3-((((1S,3aS,5aR,5bR,7aR,9S,11aR,11bR,13aR,13bR)-3a-(((2S,3S)-2-(1,1-dioxidothiomorpholino)-3-hydroxybutyl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1R,3S)-3-((((1S,3aS,5aR,5bR,7aR,9S,11aR,11bR,13aR,13bR)-3a-(((2R,3S)-2-(1,1-dioxidothiomorpholino)-3-hydroxybutyl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1R,3S)-3-((((1S,3aS,5aR,5bR,7aR,9S,11aR,11bR,13aR,13bR)-3a-(((2S,3R)-2-(1,1-dioxidothiomorpholino)-3-hydroxybutyl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1S,3R)-3-((((1R,3aS,5aR,5bR,7aR,9S,11 aR,11bR,13aR,13bR)-3a-(((2R,3R)-2-(1,1-dioxidothiomorpholino)-3-hydroxybutyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1S,3R)-3-((((1R,3aS,5aR,5bR,7aR,9S,11aR,11bR,13aR,13bR)-3a-(((2S,3S)-2-(1,1-dioxidothiomorpholino)-3-hydroxybutyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1S,3R)-3-((((1R,3aS,5aR,5bR,7aR,9S,11 aR,11bR,13aR,13bR)-3a-(((2R,3S)-2-(1,1-dioxidothiomorpholino)-3-hydroxybutyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1S,3R)-3-((((1R,3aS,5aR,5bR,7aR,9S,11aR,11bR,13aR,13bR)-3a-(((2S,3R)-2-(1,1-dioxidothiomorpholino)-3-hydroxybutyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1S,3R)-3-((((1R,3aS,5aR,5bR,7aR,9S,11 aR,11bR,13aR,13bR)-3a-(((2R,3R)-2-(1,1-dioxidothiomorpholino)-3-hydroxybutyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1S,3R)-3-((((1R,3aS,5aR,5bR,7aR,9S,11aR,11bR,13aR,13bR)-3a-(((2S,3S)-2-(1,1-dioxidothiomorpholino)-3-hydroxybutyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1S,3R)-3-((((1R,3aS,5aR,5bR,7aR,9S,11 aR,11bR,13aR,13bR)-3a-(((2R,3S)-2-(1,1-dioxidothiomorpholino)-3-hydroxybutyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1S,3R)-3-((((1R,3aS,5aR,5bR,7aR,9S,11aR,11bR,13aR,13bR)-3a-(((2S,3R)-2-(1,1-dioxidothiomorpholino)-3-hydroxybutyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1S,3R)-3-((((1S,3aS,5aR,5bR,7aR,9S,11aR,11bR,13aR,13bR)-3a-(((2R,3R)-2-(1,1-dioxidothiomorpholino)-3-hydroxybutyl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1S,3R)-3-((((1S,3aS,5aR,5bR,7aR,9S,11aR,11bR,13aR,13bR)-3a-(((2S,3S)-2-(1,1-dioxidothiomorpholino)-3-hydroxybutyl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1S,3R)-3-((((1S,3aS,5aR,5bR,7aR,9S,11aR,11bR,13aR,13bR)-3a-(((2R,3S)-2-(1,1-dioxidothiomorpholino)-3-hydroxybutyl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1S,3R)-3-((((1S,3aS,5aR,5bR,7aR,9S,11aR,11bR,13aR,13bR)-3a-(((2S,3R)-2-(1,1-dioxidothiomorpholino)-3-hydroxybutyl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((2R,3R)-2-(1,1-dioxidothiomorpholino)-3-hydroxybutyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclopropane-1-carboxylic acid,
(1S,3R)-3-((((1R,3aS,5aR,5bR,7aR,9S,11 aR,11bR,13aR,13bR)-3a-(((2R,3R)-2-(1,1-dioxidothiomorpholino)-3-hydroxybutyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclopropane-1-carboxylic acid,
(1R,3S)-3-((((1R,3aS,5aR,5bR,7aR,9S,11aR,11bR,13aR,13bR)-3a-(((2S,3S)-2-(1,1-dioxidothiomorpholino)-3-hydroxybutyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclopropane-1-carboxylic acid,
(1S,3R)-3-((((1R,3aS,5aR,5bR,7aR,9S,11aR,11bR,13aR,13bR)-3a-(((2S,3S)-2-(1,1-dioxidothiomorpholino)-3-hydroxybutyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclopropane-1-carboxylic acid,
(1S,3R)-3-(2-(((1R,3aS,5aR,5bR,7aR,9S,11 aR,11bR,13aR,13bR)-3a-(((2R,3R)-2-(1,1-dioxidothiomorpholino)-3-hydroxybutyl)amino)-5a,5b,8,8,11a-pentamethyl-1-(prop-1-en-2-yl)icosahydro-1H-cyclopenta[a]chrysen-9-yl)oxy)-2-oxoethyl)-2,2-dimethylcyclopropane-1-carboxylic acid,
(1S,3R)-3-(2-(((1R,3aS,5aR,5bR,7aR,9S,11aR,11bR,13aR,13bR)-3a-(((2S,3S)-2-(1,1-dioxidothiomorpholino)-3-hydroxybutyl)amino)-5a,5b,8,8,11a-pentamethyl-1-(prop-1-en-2-yl)icosahydro-1H-cyclopenta[a]chrysen-9-yl)oxy)-2-oxoethyl)-2,2-dimethylcyclopropane-1-carboxylic acid,
2-((1R,3S)-3-((((1R,3aS,5aR,5bR,7aR,9S,11aR,11bR,13aR,13bR)-3a-(((2R,3R)-2-(1,1-dioxidothiomorpholino)-3-hydroxybutyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclopropyl)acetic acid,
2-((1R,3S)-3-((((1R,3aS,5aR,5bR,7aR,9S,11aR,11bR,13aR,13bR)-3a-(((2S,3S)-2-(1,1-dioxidothiomorpholino)-3-hydroxybutyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclopropyl)acetic acid,
2-((1R,3S)-3-((((1R,3aS,5aR,5bR,7aR,9S,11aR,11bR,13aR,13bR)-3a-(((2R,3R)-2-(1,1-dioxidothiomorpholino)-3-hydroxybutyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclopropyl)acetic acid,
2-((1R,3S)-3-((((1R,3aS,5aR,5bR,7aR,9S,11aR,11bR,13aR,13bR)-3a-(((2S,3S)-2-(1,1-dioxidothiomorpholino)-3-hydroxybutyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclopropyl)acetic acid,
2-((1R,3S)-3-((((1S,3aS,5aR,5bR,7aR,9S,11aR,11bR,13aR,13bR)-3a-(((2R,3R)-2-(1,1-dioxidothiomorpholino)-3-hydroxybutyl)amino)-1-isopropyl-5a,5b,8,8,11a-pentamethylicosahydro-1H-cyclopenta[a]chrysen-9-yl)oxy)carbonyl)-2,2-dimethylcyclopropyl)acetic acid,
2-((1R,3S)-3-((((1S,3aS,5aR,5bR,7aR,9S,11aR,11bR,13aR,13bR)-3a-(((2S,3S)-2-(1,1-dioxidothiomorpholino)-3-hydroxybutyl)amino)-1-isopropyl-5a,5b,8,8,11a-pentamethylicosahydro-1H-cyclopenta[a]chrysen-9-yl)oxy)carbonyl)-2,2-dimethylcyclopropyl)acetic acid,
4-(((1R,3aS,5aR,5bR,7aR,9S,11aR,11bR,13aR,13bR)-3a-(((2R,3R)-2-(1,1-dioxidothiomorpholino)-3-hydroxybutyl)amino)-5a,5b,8,8,11a-pentamethyl-1-(prop-1-en-2-yl)icosahydro-1H-cyclopenta[a]chrysen-9-yl)oxy)-2,2-dimethyl-4-oxobutanoic acid,
4-(((1R,3aS,5aR,5bR,7aR,9S,11aR,11bR,13aR,13bR)-3a-(((2S,3S)-2-(1,1-dioxidothiomorpholino)-3-hydroxybutyl)amino)-5a,5b,8,8,11a-pentamethyl-1-(prop-1-en-2-yl)icosahydro-1H-cyclopenta[a]chrysen-9-yl)oxy)-2,2-dimethyl-4-oxobutanoic acid,
4-(((1R,3aS,5aR,5bR,7aR,9S,11aR,11bR,13aR,13bR)-3a-(((2R,3R)-2-(1,1-dioxidothiomorpholino)-3-hydroxybutyl)amino)-5a,5b,8,8,11a-pentamethyl-1-(1-methylcyclopropyl)icosahydro-1H-cyclopenta[a]chrysen-9-yl)oxy)-2,2-dimethyl-4-oxobutanoic acid,
4-(((1R,3aS,5aR,5bR,7aR,9S,11aR,11bR,13aR,13bR)-3a-(((2S,3S)-2-(1,1-dioxidothiomorpholino)-3-hydroxybutyl)amino)-5a,5b,8,8,11a-pentamethyl-1-(1-methylcyclopropyl)icosahydro-1H-cyclopenta[a]chrysen-9-yl)oxy)-2,2-dimethyl-4-oxobutanoic acid,
4-(((1S,3aS,5aR,5bR,7aR,9S,11aR,11bR,13aR,13bR)-3a-(((2R,3R)-2-(1,1-dioxidothiomorpholino)-3-hydroxybutyl)amino)-1-isopropyl-5a,5b,8,8,11a-pentamethylicosahydro-1H-cyclopenta[a]chrysen-9-yl)oxy)-2,2-dimethyl-4-oxobutanoic acid,
4-(((1S,3aS,5aR,5bR,7aR,9S,11aR,11bR,13aR,13bR)-3a-(((2S,3S)-2-(1,1-dioxidothiomorpholino)-3-hydroxybutyl)amino)-1-isopropyl-5a,5b,8,8,11a-pentamethylicosahydro-1H-cyclopenta[a]chrysen-9-yl)oxy)-2,2-dimethyl-4-oxobutanoic acid,
5-(((1R,3aS,5aR,5bR,7aR,9S,11aR,11bR,13aR,13bR)-3a-(((2R,3R)-2-(1,1-dioxidothiomorpholino)-3-hydroxybutyl)amino)-5a,5b,8,8,11a-pentamethyl-1-(prop-1-en-2-yl)icosahydro-1H-cyclopenta[a]chrysen-9-yl)oxy)-3,3-dimethyl-5-oxopentanoic acid,
5-(((1R,3aS,5aR,5bR,7aR,9S,11aR,11bR,13aR,13bR)-3a-(((2S,3S)-2-(1,1-dioxidothiomorpholino)-3-hydroxybutyl)amino)-5a,5b,8,8,11a-pentamethyl-1-(prop-1-en-2-yl)icosahydro-1H-cyclopenta[a]chrysen-9-yl)oxy)-3,3-dimethyl-5-oxopentanoic acid,
5-(((1R,3aS,5aR,5bR,7aR,9S,11aR,11bR,13aR,13bR)-3a-(((2R,3R)-2-(1,1-dioxidothiomorpholino)-3-hydroxybutyl)amino)-5a,5b,8,8,11a-pentamethyl-1-(1-methylcyclopropyl)icosahydro-1H-cyclopenta[a]chrysen-9-yl)oxy)-3,3-dimethyl-5-oxopentanoic acid,
5-(((1R,3aS,5aR,5bR,7aR,9S,11aR,11bR,13aR,13bR)-3a-(((2S,3S)-2-(1,1-dioxidothiomorpholino)-3-hydroxybutyl)amino)-5a,5b,8,8,11a-pentamethyl-1-(1-methylcyclopropyl)icosahydro-1H-cyclopenta[a]chrysen-9-yl)oxy)-3,3-dimethyl-5-oxopentanoic acid,
5-(((1S,3aS,5aR,5bR,7aR,9S,11aR,11bR,13aR,13bR)-3a-(((2R,3R)-2-(1,1-dioxidothiomorpholino)-3-hydroxybutyl)amino)-1-isopropyl-5a,5b,8,8,11a-pentamethylicosahydro-1H-cyclopenta[a]chrysen-9-yl)oxy)-3,3-dimethyl-5-oxopentanoic acid,
5-(((1S,3aS,5aR,5bR,7aR,9S,11aR,11bR,13aR,13bR)-3a-(((2S,3S)-2-(1,1-dioxidothiomorpholino)-3-hydroxybutyl)amino)-1-isopropyl-5a,5b,8,8,11a-pentamethylicosahydro-1H-cyclopenta[a]chrysen-9-yl)oxy)-3,3-dimethyl-5-oxopentanoic acid,
4-(((1R,3aS,5aR,5bR,7aR,9S,11aR,11bR,13aR,13bR)-3a-(((2R,3R)-3-((dimethoxyphosphoryl)methoxy)-2-(1,1-dioxidothiomorpholino)butyl)amino)-5a,5b,8,8,11a-pentamethyl-1-(prop-1-en-2-yl)icosahydro-1H-cyclopenta[a]chrysen-9-yl)oxy)-2,2-dimethyl-4-oxobutanoic acid,
4-(((1R,3aS,5aR,5bR,7aR,9S,11aR,11bR,13aR,13bR)-3a-(((2R,3R)-2-(1,1-dioxidothiomorpholino)-3-(phosphonomethoxy)butyl)amino)-5a,5b,8,8,11a-pentamethyl-1-(prop-1-en-2-yl)icosahydro-1H-cyclopenta[a]chrysen-9-yl)oxy)-2,2-dimethyl-4-oxobutanoic acid,
(1R,3S)-3-((((1R,3aS,5aR,5bR,7aR,9S,11aR,11bR,13aR,13bR)-3a-(((2R,3R)-3-((dimethoxyphosphoryl)methoxy)-2-(1,1-dioxidothiomorpholino)butyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((2R,3R)-2-(1,1-dioxidothiomorpholino)-3-(phosphonomethoxy)butyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((2R,3R)-3-((dimethoxyphosphoryl)methoxy)-2-(1,1-dioxidothiomorpholino)butyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((2R,3R)-2-(1,1-dioxidothiomorpholino)-3-(phosphonomethoxy)butyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1R,3S)-3-((((1S,3aS,5aR,5bR,7aR,9S,11aR,11bR,13aR,13bR)-3a-(((2R,3R)-3-((dimethoxyphosphoryl)methoxy)-2-(1,1-dioxidothiomorpholino)butyl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1R,3S)-3-((((1S,3aS,5aR,5bR,7aR,9S,11aR,11bR,13aR,13bR)-3a-(((2R,3R)-2-(1,1-dioxidothiomorpholino)-3-(phosphonomethoxy)butyl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
5-(((1R,3aS,5aR,5bR,7aR,9S,11aR,11bR,13aR,13bR)-3a-(((2R,3R)-3-((dimethoxyphosphoryl)methoxy)-2-(1,1-dioxidothiomorpholino)butyl)amino)-5a,5b,8,8,11a-pentamethyl-1-(prop-1-en-2-yl)icosahydro-1H-cyclopenta[a]chrysen-9-yl)oxy)-3,3-dimethyl-5-oxopentanoic acid,
5-(((1R,3aS,5aR,5bR,7aR,9S,11aR,11bR,13aR,13bR)-3a-(((2R,3R)-2-(1,1-dioxidothiomorpholino)-3-(phosphonomethoxy)butyl)amino)-5a,5b,8,8,11a-pentamethyl-1-(prop-1-en-2-yl)icosahydro-1H-cyclopenta[a]chrysen-9-yl)oxy)-3,3-dimethyl-5-oxopentanoic acid,
5-(((1R,3aS,5aR,5bR,7aR,9S,11aR,11bR,13aR,13bR)-3a-(((2R,3R)-3-((dimethoxyphosphoryl)methoxy)-2-(1,1-dioxidothiomorpholino)butyl)amino)-5a,5b,8,8,11a-pentamethyl-1-(1-methylcyclopropyl)icosahydro-1H-cyclopenta[a]chrysen-9-yl)oxy)-3,3-dimethyl-5-oxopentanoic acid,
5-(((1R,3aS,5aR,5bR,7aR,9S,11aR,11bR,13aR,13bR)-3a-(((2R,3R)-2-(1,1-dioxidothiomorpholino)-3-(phosphonomethoxy)butyl)amino)-5a,5b,8,8,11a-pentamethyl-1-(1-methylcyclopropyl)icosahydro-1H-cyclopenta[a]chrysen-9-yl)oxy)-3,3-dimethyl-5-oxopentanoic acid,
5-(((1S,3aS,5aR,5bR,7aR,9S,11aR,11bR,13aR,13bR)-3a-(((2R,3R)-3-((dimethoxyphosphoryl)methoxy)-2-(1,1-dioxidothiomorpholino)butyl)amino)-1-isopropyl-5a,5b,8,8,11a-pentamethylicosahydro-1H-cyclopenta[a]chrysen-9-yl)oxy)-3,3-dimethyl-5-oxopentanoic acid,
5-(((1S,3aS,5aR,5bR,7aR,9S,11aR,11bR,13aR,13bR)-3a-(((2R,3R)-2-(1,1-dioxidothiomorpholino)-3-(phosphonomethoxy)butyl)amino)-1-isopropyl-5a,5b,8,8,11a-pentamethylicosahydro-1H-cyclopenta[a]chrysen-9-yl)oxy)-3,3-dimethyl-5-oxopentanoic acid,
2-((1R,3S)-3-((((1R,3aS,5aR,5bR,7aR,9S,11aR,11bR,13aR,13bR)-3a-(((2R,3R)-3-((dimethoxyphosphoryl)methoxy)-2-(1,1-dioxidothiomorpholino)butyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclopropyl)acetic acid,
2-((1R,3S)-3-((((1R,3aS,5aR,5bR,7aR,9S,11aR,11bR,13aR,13bR)-3a-(((2R,3R)-2-(1,1-dioxidothiomorpholino)-3-(phosphonomethoxy)butyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclopropyl)acetic acid,
2-((1R,3S)-3-((((1R,3aS,5aR,5bR,7aR,9S,11aR,11bR,13aR,13bR)-3a-(((2R,3R)-3-((dimethoxyphosphoryl)methoxy)-2-(1,1-dioxidothiomorpholino)butyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclopropyl)acetic acid,
2-((1R,3S)-3-((((1R,3aS,5aR,5bR,7aR,9S,11aR,11bR,13aR,13bR)-3a-(((2R,3R)-2-(1,1-dioxidothiomorpholino)-3-(phosphonomethoxy)butyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclopropyl)acetic acid,
2-((1R,3S)-3-((((1S,3aS,5aR,5bR,7aR,9S,11aR,11bR,13aR,13bR)-3a-(((2R,3R)-3-((dimethoxyphosphoryl)methoxy)-2-(1,1-dioxidothiomorpholino)butyl)amino)-1-isopropyl-5a,5b,8,8,11a-pentamethylicosahydro-1H-cyclopenta[a]chrysen-9-yl)oxy)carbonyl)-2,2-dimethylcyclopropyl)acetic acid,
2-((1R,3S)-3-((((1S,3aS,5aR,5bR,7aR,9S,11aR,11bR,13aR,13bR)-3a-(((2R,3R)-2-(1,1-dioxidothiomorpholino)-3-(phosphonomethoxy)butyl)amino)-1-isopropyl-5a,5b,8,8,11a-pentamethylicosahydro-1H-cyclopenta[a]chrysen-9-yl)oxy)carbonyl)-2,2-dimethylcyclopropyl)acetic acid,
4-(((1R,3aS,5aR,5bR,7aR,9S,11aR,11bR,13aR,13bR)-3a-(((12R,13R)-13-(1,1-dioxidothiomorpholino)-12-methyl-2,5,8,11-tetraoxatetradecan-14-yl)amino)-5a,5b,8,8,11a-pentamethyl-1-(prop-1-en-2-yl)icosahydro-1H-cyclopenta[a]chrysen-9-yl)oxy)-2,2-dimethyl-4-oxobutanoic acid,
4-(((1R,3aS,5aR,5bR,7aR,9S,11aR,11bR,13aR,13bR)-3a-(((12R,13R)-13-(1,1-dioxidothiomorpholino)-12-methyl-2,5,8,11-tetraoxatetradecan-14-yl)amino)-5a,5b,8,8,11a-pentamethyl-1-(1-methylcyclopropyl)icosahydro-1H-cyclopenta[a]chrysen-9-yl)oxy)-2,2-dimethyl-4-oxobutanoic acid,
4-(((1S,3aS,5aR,5bR,7aR,9S,11aR,11bR,13aR,13bR)-3a-(((12R,13R)-13-(1,1-dioxidothiomorpholino)-12-methyl-2,5,8,11-tetraoxatetradecan-14-yl)amino)-1-isopropyl-5a,5b,8,8,11a-pentamethylicosahydro-1H-cyclopenta[a]chrysen-9-yl)oxy)-2,2-dimethyl-4-oxobutanoic acid,
5-(((1R,3aS,5aR,5bR,7aR,9S,11aR,11bR,13aR,13bR)-3a-(((12R,13R)-13-(1,1-dioxidothiomorpholino)-12-methyl-2,5,8,11-tetraoxatetradecan-14-yl)amino)-5a,5b,8,8,11a-pentamethyl-1-(prop-1-en-2-yl)icosahydro-1H-cyclopenta[a]chrysen-9-yl)oxy)-3,3-dimethyl-5-oxopentanoic acid,
5-(((1R,3aS,5aR,5bR,7aR,9S,11aR,11bR,13aR,13bR)-3a-(((12R,13R)-13-(1,1-dioxidothiomorpholino)-12-methyl-2,5,8,11-tetraoxatetradecan-14-yl)amino)-5a,5b,8,8,11a-pentamethyl-1-(1-methylcyclopropyl)icosahydro-1H-cyclopenta[a]chrysen-9-yl)oxy)-3,3-dimethyl-5-oxopentanoic acid,
5-(((1S,3aS,5aR,5bR,7aR,9S,11aR,11bR,13aR,13bR)-3a-(((12R,13R)-13-(1,1-dioxidothiomorpholino)-12-methyl-2,5,8,11-tetraoxatetradecan-14-yl)amino)-1-isopropyl-5a,5b,8,8,11a-pentamethylicosahydro-1H-cyclopenta[a]chrysen-9-yl)oxy)-3,3-dimethyl-5-oxopentanoic acid,
4-(((1R,3aS,5aR,5bR,7aR,9S,11aR,11bR,13aR,13bR)-3a-(((15R,16R)-16-(1,1-dioxidothiomorpholino)-15-methyl-13-oxo-2,5,8,11,14-pentaoxaheptadecan-17-yl)amino)-5a,5b,8,8,11a-pentamethyl-1-(prop-1-en-2-yl)icosahydro-1H-cyclopenta[a]chrysen-9-yl)oxy)-2,2-dimethyl-4-oxobutanoic acid,
4-(((1R,3aS,5aR,5bR,7aR,9S,11aR,11bR,13aR,13bR)-3a-(((15R,16R)-16-(1,1-dioxidothiomorpholino)-15-methyl-13-oxo-2,5,8,11,14-pentaoxaheptadecan-17-yl)amino)-5a,5b,8,8,11a-pentamethyl-1-(1-methylcyclopropyl)icosahydro-1H-cyclopenta[a]chrysen-9-yl)oxy)-2,2-dimethyl-4-oxobutanoic acid,
4-(((1S,3aS,5aR,5bR,7aR,9S,11aR,11bR,13aR,13bR)-3a-(((15R,16R)-16-(1,1-dioxidothiomorpholino)-15-methyl-13-oxo-2,5, 8,11,14-pentaoxaheptadecan-17-yl)amino)-1-isopropyl-5a,5b,8,8,11a-pentamethylicosahydro-1H-cyclopenta[a]chrysen-9-yl)oxy)-2,2-dimethyl-4-oxobutanoic acid,
5-(((1R,3aS,5aR,5bR,7aR,9S,11aR,11bR,13aR,13bR)-3a-(((15R,16R)-16-(1,1-dioxidothiomorpholino)-15-methyl-13-oxo-2,5,8,11,14-pentaoxaheptadecan-17-yl)amino)-5a,5b,8,8,11a-pentamethyl-1-(prop-1-en-2-yl)icosahydro-1H-cyclopenta[a]chrysen-9-yl)oxy)-3,3-dimethyl-5-oxopentanoic acid,
5-(((1R,3aS,5aR,5bR,7aR,9S,11aR,11bR,13aR,13bR)-3a-(((15R,16R)-16-(1,1-dioxidothiomorpholino)-15-methyl-13-oxo-2,5,8,11,14-pentaoxaheptadecan-17-yl)amino)-5a,5b,8,8,11a-pentamethyl-1-(1-methylcyclopropyl)icosahydro-1H-cyclopenta[a]chrysen-9-yl)oxy)-3,3-dimethyl-5-oxopentanoic acid,
5-(((1S,3aS,5aR,5bR,7aR,9S,11aR,11bR,13aR,13bR)-3a-(((15R,16R)-16-(1,1-dioxidothiomorpholino)-15-methyl-13-oxo-2,5, 8,11,14-pentaoxaheptadecan-17-yl)amino)-1-isopropyl-5a,5b,8,8,11a-pentamethylicosahydro-1H-cyclopenta[a]chrysen-9-yl)oxy)-3,3-dimethyl-5-oxopentanoic acid,
(1R,3S)-3-((((1R,3aS,5aR,5bR,7aR,9S,11aR,11bR,13aR,13bR)-3a-(((S)-3-amino-2-(1,1-dioxidothiomorpholino)propyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((S)-3-amino-2-(1,1-dioxidothiomorpholino)propyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1R,3S)-3-((((1S,3aS,5aR,5bR,7aR,9S,11aR,11bR,13aR,13bR)-3a-(((S)-3-amino-2-(1,1-dioxidothiomorpholino)propyl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((2R,3R)-3-amino-2-(1,1-dioxidothiomorpholino)butyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((2R,3R)-3-amino-2-(1,1-dioxidothiomorpholino)butyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1R,3S)-3-((((1S,3aS,5aR,5bR,7aR,9S,11aR,11bR,13aR,13bR)-3a-(((2R,3R)-3-amino-2-(1,1-dioxidothiomorpholino)butyl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((R)-3-((tert-butoxycarbonyl)amino)-2-(1,1-dioxidothiomorpholino)propyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((R)-3-((tert-butoxycarbonyl)amino)-2-(1,1-dioxidothiomorpholino)propyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1R,3S)-3-((((1S,3aS,5aR,5bR,7aR,9S,11aR,11bR,13aR,13bR)-3a-(((R)-3-((tert-butoxycarbonyl)amino)-2-(1,1-dioxidothiomorpholino)propyl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((2R,3R)-3-((tert-butoxycarbonyl)amino)-2-(1,1-dioxidothiomorpholino)butyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((2R,3R)-3-((tert-butoxycarbonyl)amino)-2-(1,1-dioxidothiomorpholino)butyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1R,3S)-3-((((1S,3aS,5aR,5bR,7aR,9S,11aR,11bR,13aR,13bR)-3a-(((2R,3R)-3-((tert-butoxycarbonyl)amino)-2-(1,1-dioxidothiomorpholino)butyl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((R)-2-(1,1-dioxidothiomorpholino)-3-((ethoxycarbonyl)amino)propyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((R)-2-(1,1-dioxidothiomorpholino)-3-((ethoxycarbonyl)amino)propyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1R,3S)-3-((((1S,3aS,5aR,5bR,7aR,9S,11aR,11bR,13aR,13bR)-3a-(((R)-2-(1,1-dioxidothiomorpholino)-3-((ethoxycarbonyl)amino)propyl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((2R,3R)-2-(1,1-dioxidothiomorpholino)-3-((ethoxycarbonyl)amino)butyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((2R,3R)-2-(1,1-dioxidothiomorpholino)-3-((ethoxycarbonyl)amino)butyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1R,3S)-3-((((1S,3aS,5aR,5bR,7aR,9S,11aR,11bR,13aR,13bR)-3a-(((2R,3R)-2-(1,1-dioxidothiomorpholino)-3-((ethoxycarbonyl)amino)butyl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((R)-2-(1,1-dioxidothiomorpholino)-3-((isopropoxycarbonyl)amino)propyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((R)-2-(1,1-dioxidothiomorpholino)-3-((isopropoxycarbonyl)amino)propyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1R,3S)-3-((((1S,3aS,5aR,5bR,7aR,9S,11aR,11bR,13aR,13bR)-3a-(((R)-2-(1,1-dioxidothiomorpholino)-3-((isopropoxycarbonyl)amino)propyl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((2R,3R)-2-(1,1-dioxidothiomorpholino)-3-((isopropoxycarbonyl)amino)butyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((2R,3R)-2-(1,1-dioxidothiomorpholino)-3-((isopropoxycarbonyl)amino)butyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1R,3S)-3-((((1S,3aS,5aR,5bR,7aR,9S,11aR,11bR,13aR,13bR)-3a-(((2R,3R)-2-(1,1-dioxidothiomorpholino)-3-((isopropoxycarbonyl)amino)butyl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((R)-2-(1,1-dioxidothiomorpholino)-3-(((2-methoxyethoxy)carbonyl)amino)propyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((R)-2-(1,1-dioxidothiomorpholino)-3-(((2-methoxyethoxy)carbonyl)amino)propyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1R,3S)-3-((((1S,3aS,5aR,5bR,7aR,9S,11aR,11bR,13aR,13bR)-3a-(((R)-2-(1,1-dioxidothiomorpholino)-3-(((2-methoxyethoxy)carbonyl)amino)propyl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((2R,3R)-2-(1,1-dioxidothiomorpholino)-3-(((2-methoxyethoxy)carbonyl)amino)butyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((2R,3R)-2-(1,1-dioxidothiomorpholino)-3-(((2-methoxyethoxy)carbonyl)amino)butyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1R,3S)-3-((((1S,3aS,5aR,5bR,7aR,9S,11aR,11bR,13aR,13bR)-3a-(((2R,3R)-2-(1,1-dioxidothiomorpholino)-3-(((2-methoxyethoxy)carbonyl)amino)butyl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((R)-12-(1,1-dioxidothiomorpholino)-9-oxo-2,5, 8-trioxa-10-azatridecan-13-yl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((R)-12-(1,1-dioxidothiomorpholino)-9-oxo-2,5, 8-trioxa-10-azatridecan-13-yl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1R,3S)-3-((((1S,3aS,5aR,5bR,7aR,9S,11aR,11bR,13aR,13bR)-3a-(((R)-12-(1,1-dioxidothiomorpholino)-9-oxo-2,5,8-trioxa-10-azatridecan-13-yl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((11R,12R)-12-(1,1-dioxidothiomorpholino)-11-methyl-9-oxo-2,5,8-trioxa-10-azatridecan-13-yl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((11R,12R)-12-(1,1-dioxidothiomorpholino)-11-methyl-9-oxo-2,5,8-trioxa-10-azatridecan-13-yl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1R,3S)-3-((((1S,3aS,5aR,5bR,7aR,9S,11aR,11bR,13aR,13bR)-3a-(((11R,12R)-12-(1,1-dioxidothiomorpholino)-11-methyl-9-oxo-2,5,8-trioxa-10-azatridecan-13-yl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((R)-2-(1,1-dioxidothiomorpholino)-3-((1-methylethyl)sulfonamido)propyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((R)-2-(1,1-dioxidothiomorpholino)-3-((1-methylethyl)sulfonamido)propyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1R,3S)-3-((((1S,3aS,5aR,5bR,7aR,9S,11aR,11bR,13aR,13bR)-3a-(((R)-2-(1,1-dioxidothiomorpholino)-3-((1-methylethyl)sulfonamido)propyl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((2R,3R)-2-(1,1-dioxidothiomorpholino)-3-((1-methylethyl)sulfonamido)butyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((2R,3R)-2-(1,1-dioxidothiomorpholino)-3-((1-methylethyl)sulfonamido)butyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1R,3S)-3-((((1S,3aS,5aR,5bR,7aR,9S,11aR,11bR,13aR,13bR)-3a-(((2R,3R)-2-(1,1-dioxidothiomorpholino)-3-((1-methylethyl)sulfonamido)butyl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((R)-2-(1,1-dioxidothiomorpholino)-3-(methylthio)propyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((R)-2-(1,1-dioxidothiomorpholino)-3-(methylthio)propyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((S)-2-(1,1-dioxidothiomorpholino)-4-(methylthio)butyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((S)-2-(1,1-dioxidothiomorpholino)-4-(methylthio)butyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
4-(((1R,3aS,5aR,5bR,7aR,9S,11aR,11bR,13aR,13bR)-3a-(((S)-3-amino-2-(1,1-dioxidothiomorpholino)propyl)amino)-5a,5b,8,8,11a-pentamethyl-1-(prop-1-en-2-yl)icosahydro-1H-cyclopenta[a]chrysen-9-yl)oxy)-2,2-dimethyl-4-oxobutanoic acid,
5-(((1R,3aS,5aR,5bR,7aR,9S,11aR,11bR,13aR,13bR)-3a-(((S)-3-amino-2-(1,1-dioxidothiomorpholino)propyl)amino)-5a,5b,8,8,11a-pentamethyl-1-(prop-1-en-2-yl)icosahydro-1H-cyclopenta[a]chrysen-9-yl)oxy)-3,3-dimethyl-5-oxopentanoic acid,
4-(((1R,3aS,5aR,5bR,7aR,9S,11aR,11bR,13aR,13bR)-3a-(((2R,3R)-3-amino-2-(1,1-dioxidothiomorpholino)butyl)amino)-5a,5b,8,8,11a-pentamethyl-1-(prop-1-en-2-yl)icosahydro-1H-cyclopenta[a]chrysen-9-yl)oxy)-2,2-dimethyl-4-oxobutanoic acid,
5-(((1R,3aS,5aR,5bR,7aR,9S,11aR,11bR,13aR,13bR)-3a-(((2R,3R)-3-amino-2-(1,1-dioxidothiomorpholino)butyl)amino)-5a,5b,8,8,11a-pentamethyl-1-(prop-1-en-2-yl)icosahydro-1H-cyclopenta[a]chrysen-9-yl)oxy)-3,3-dimethyl-5-oxopentanoic acid,
4-(((1R,3aS,5aR,5bR,7aR,9S,11aR,11bR,13aR,13bR)-3a-(((R)-2-(1,1-dioxidothiomorpholino)-3-((ethoxycarbonyl)amino)propyl)amino)-5a,5b,8,8,11a-pentamethyl-1-(prop-1-en-2-yl)icosahydro-1H-cyclopenta[a]chrysen-9-yl)oxy)-2,2-dimethyl-4-oxobutanoic acid,
5-(((1R,3aS,5aR,5bR,7aR,9S,11aR,11bR,13aR,13bR)-3a-(((R)-2-(1,1-dioxidothiomorpholino)-3-((ethoxycarbonyl)amino)propyl)amino)-5a,5b,8,8,11a-pentamethyl-1-(prop-1-en-2-yl)icosahydro-1H-cyclopenta[a]chrysen-9-yl)oxy)-3,3-dimethyl-5-oxopentanoic acid,
4-(((1R,3aS,5aR,5bR,7aR,9S,11aR,11bR,13aR,13bR)-3a-(((2R,3R)-2-(1,1-dioxidothiomorpholino)-3-((ethoxycarbonyl)amino)butyl)amino)-5a,5b,8,8,11a-pentamethyl-1-(prop-1-en-2-yl)icosahydro-1H-cyclopenta[a]chrysen-9-yl)oxy)-2,2-dimethyl-4-oxobutanoic acid,
5-(((1R,3aS,5aR,5bR,7aR,9S,11aR,11bR,13aR,13bR)-3a-(((2R,3R)-2-(1,1-dioxidothiomorpholino)-3-((ethoxycarbonyl)amino)butyl)amino)-5a,5b,8,8,11a-pentamethyl-1-(prop-1-en-2-yl)icosahydro-1H-cyclopenta[a]chrysen-9-yl)oxy)-3,3-dimethyl-5-oxopentanoic acid,
4-(((1R,3aS,5aR,5bR,7aR,9S,11aR,11bR,13aR,13bR)-3a-(((R)-2-(1,1-dioxidothiomorpholino)-3-((1-methylethyl)sulfonamido)propyl)amino)-5a,5b,8,8,11a-pentamethyl-1-(prop-1-en-2-yl)icosahydro-1H-cyclopenta[a]chrysen-9-yl)oxy)-2,2-dimethyl-4-oxobutanoic acid,
5-(((1R,3aS,5aR,5bR,7aR,9S,11aR,11bR,13aR,13bR)-3a-(((R)-2-(1,1-dioxidothiomorpholino)-3-((1-methylethyl)sulfonamido)propyl)amino)-5a,5b,8,8,11a-pentamethyl-1-(prop-1-en-2-yl)icosahydro-1H-cyclopenta[a]chrysen-9-yl)oxy)-3,3-dimethyl-5-oxopentanoic acid,
4-(((1R,3aS,5aR,5bR,7aR,9S,11aR,11bR,13aR,13bR)-3a-(((2R,3R)-2-(1,1-dioxidothiomorpholino)-3-((1-methylethyl)sulfonamido)butyl)amino)-5a,5b,8,8,11a-pentamethyl-1-(prop-1-en-2-yl)icosahydro-1H-cyclopenta[a]chrysen-9-yl)oxy)-2,2-dimethyl-4-oxobutanoic acid,
5-(((1R,3aS,5aR,5bR,7aR,9S,11aR,11bR,13aR,13bR)-3a-(((2R,3R)-2-(1,1-dioxidothiomorpholino)-3-((1-methylethyl)sulfonamido)butyl)amino)-5a,5b,8,8,11a-pentamethyl-1-(prop-1-en-2-yl)icosahydro-1H-cyclopenta[a]chrysen-9-yl)oxy)-3,3-dimethyl-5-oxopentanoic acid,
4-(((1R,3aS,5aR,5bR,7aR,9S,11aR,11bR,13aR,13bR)-3a-(((R)-2-(1,1-dioxidothiomorpholino)-3-(methylthio)propyl)amino)-5a,5b,8,8,11a-pentamethyl-1-(prop-1-en-2-yl)icosahydro-1H-cyclopenta[a]chrysen-9-yl)oxy)-2,2-dimethyl-4-oxobutanoic acid,
5-(((1R,3aS,5aR,5bR,7aR,9S,11aR,11bR,13aR,13bR)-3a-(((R)-2-(1,1-dioxidothiomorpholino)-3-(methylthio)propyl)amino)-5a,5b,8,8,11a-pentamethyl-1-(prop-1-en-2-yl)icosahydro-1H-cyclopenta[a]chrysen-9-yl)oxy)-3,3-dimethyl-5-oxopentanoic acid,
4-(((1R,3aS,5aR,5bR,7aR,9S,11aR,11bR,13aR,13bR)-3a-(((S)-2-(1,1-dioxidothiomorpholino)-4-(methylthio)butyl)amino)-5a,5b,8,8,11a-pentamethyl-1-(prop-1-en-2-yl)icosahydro-1H-cyclopenta[a]chrysen-9-yl)oxy)-2,2-dimethyl-4-oxobutanoic acid,
5-(((1R,3aS,5aR,5bR,7aR,9S,11aR,11bR,13aR,13bR)-3a-(((S)-2-(1,1-dioxidothiomorpholino)-4-(methylthio)butyl)amino)-5a,5b,8,8,11a-pentamethyl-1-(prop-1-en-2-yl)icosahydro-1H-cyclopenta[a]chrysen-9-yl)oxy)-3,3-dimethyl-5-oxopentanoic acid,
4-(((1R,3aS,5aR,5bR,7aR,9S,11aR,11bR,13aR,13bR)-3a-(((S)-2-(1,1-dioxidothiomorpholino)-3-(methylthio)propyl)amino)-5a,5b,8,8,11a-pentamethyl-1-(prop-1-en-2-yl)icosahydro-1H-cyclopenta[a]chrysen-9-yl)oxy)-2,2-dimethyl-4-oxobutanoic acid,
4-(((1R,3aS,5aR,5bR,7aR,9S,11aR,11bR,13aR,13bR)-3a-(((S)-2-(1,1-dioxidothiomorpholino)-3-(methylthio)propyl)amino)-5a,5b,8,8,11a-pentamethyl-1-(1-methylcyclopropyl)icosahydro-1H-cyclopenta[a]chrysen-9-yl)oxy)-2,2-dimethyl-4-oxobutanoic acid,
4-(((1S,3aS,5aR,5bR,7aR,9S,11aR,11bR,13aR,13bR)-3a-(((S)-2-(1,1-dioxidothiomorpholino)-3-(methylthio)propyl)amino)-1-isopropyl-5a,5b,8,8,11a-pentamethylicosahydro-1H-cyclopenta[a]chrysen-9-yl)oxy)-2,2-dimethyl-4-oxobutanoic acid,
4-(((1R,3aS,5aR,5bR,7aR,9S,11aR,11bR,13aR,13bR)-3a-(((R)-2-(1,1-dioxidothiomorpholino)-4-(methylthio)butyl)amino)-5a,5b,8,8,11a-pentamethyl-1-(prop-1-en-2-yl)icosahydro-1H-cyclopenta[a]chrysen-9-yl)oxy)-2,2-dimethyl-4-oxobutanoic acid,
4-(((1R,3aS,5aR,5bR,7aR,9S,11aR,11bR,13aR,13bR)-3a-(((R)-2-(1,1-dioxidothiomorpholino)-4-(methylthio)butyl)amino)-5a,5b,8,8,11a-pentamethyl-1-(1-methylcyclopropyl)icosahydro-1H-cyclopenta[a]chrysen-9-yl)oxy)-2,2-dimethyl-4-oxobutanoic acid,
4-(((1S,3aS,5aR,5bR,7aR,9S,11aR,11bR,13aR,13bR)-3a-(((R)-2-(1,1-dioxidothiomorpholino)-4-(methylthio)butyl)amino)-1-isopropyl-5a,5b,8,8,11a-pentamethylicosahydro-1H-cyclopenta[a]chrysen-9-yl)oxy)-2,2-dimethyl-4-oxobutanoic acid,
5-(((1R,3aS,5aR,5bR,7aR,9S,11aR,11bR,13aR,13bR)-3a-(((R)-2-(1,1-dioxidothiomorpholino)-4-(methylthio)butyl)amino)-5a,5b,8,8,11a-pentamethyl-1-(prop-1-en-2-yl)icosahydro-1H-cyclopenta[a]chrysen-9-yl)oxy)-3,3-dimethyl-5-oxopentanoic acid,
5-(((1R,3aS,5aR,5bR,7aR,9S,11aR,11bR,13aR,13bR)-3a-(((R)-2-(1,1-dioxidothiomorpholino)-4-(methylthio)butyl)amino)-5a,5b,8,8,11a-pentamethyl-1-(1-methylcyclopropyl)icosahydro-1H-cyclopenta[a]chrysen-9-yl)oxy)-3,3-dimethyl-5-oxopentanoic acid,
5-(((1S,3aS,5aR,5bR,7aR,9S,11aR,11bR,13aR,13bR)-3a-(((R)-2-(1,1-dioxidothiomorpholino)-4-(methylthio)butyl)amino)-1-isopropyl-5a,5b,8,8,11a-pentamethylicosahydro-1H-cyclopenta[a]chrysen-9-yl)oxy)-3,3-dimethyl-5-oxopentanoic acid,
(1R,3S)-3-((((1R,3aS,5aR,5bR,7aR,9S,11aR,11bR,13aR,13bR)-3a-(((R)-2-(1,1-dioxidothiomorpholino)-4-(methylthio)butyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1S,3R)-3-((((1R,3aS,5aR,5bR,7aR,9S,11 aR,11bR,13aR,13bR)-3a-(((R)-2-(1,1-dioxidothiomorpholino)-4-(methylthio)butyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
2-((1R,3S)-3-((((1R,3aS,5aR,5bR,7aR,9S,11aR,11bR,13aR,13bR)-3a-(((R)-2-(1,1-dioxidothiomorpholino)-4-(methylthio)butyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclopropyl)acetic acid,
2-((1R,3S)-3-((((1R,3aS,5aR,5bR,7aR,9S,11aR,11bR,13aR,13bR)-3a-(((S)-2-(1,1-dioxidothiomorpholino)-4-(methylthio)butyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclopropyl)acetic acid,
(1R,3S)-3-((((1R,3aS,5aR,5bR,7aR,9S,11aR,11bR,13aR,13bR)-3a-(((R)-2-(1,1-dioxidothiomorpholino)-3-hydroxypropyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclopropane-1-carboxylic acid,
(1R,3S)-3-((((1R,3aS,5aR,5bR,7aR,9S,11aR,11bR,13aR,13bR)-3a-(((S)-2-(1,1-dioxidothiomorpholino)-3-hydroxypropyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclopropane-1-carboxylic acid,
(1R,3S)-3-((((1R,3aS,5aR,5bR,7aR,9S,11aR,11bR,13aR,13bR)-3a-(((2R,3S)-2-(1,1-dioxidothiomorpholino)-3-fluorobutyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclopropane-1-carboxylic acid,
(1S,3R)-3-((((1R,3aS,5aR,5bR,7aR,9S,11 aR,11bR,13aR,13bR)-3a-(((2R,3S)-2-(1,1-dioxidothiomorpholino)-3-fluorobutyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclopropane-1-carboxylic acid,
(1R,3S)-3-((((1R,3aS,5aR,5bR,7aR,9S,11aR,11bR,13aR,13bR)-3a-(((R)-2-(1,1-dioxidothiomorpholino)-3-fluoropropyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclopropane-1-carboxylic acid,
(1R,3S)-3-((((1R,3aS,5aR,5bR,7aR,9S,11aR,11bR,13aR,13bR)-3a-(((S)-2-(1,1-dioxidothiomorpholino)-3-fluoropropyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclopropane-1-carboxylic acid,
(1R,3S)-3-((((1R,3aS,5aR,5bR,7aR,9S,11aR,11bR,13aR,13bR)-3a-(((S)-3-amino-2-(1,1-dioxidothiomorpholino)propyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclopropane-1-carboxylic acid,
(1R,3S)-3-((((1R,3aS,5aR,5bR,7aR,9S,11aR,11bR,13aR,13bR)-3a-(((R)-3-amino-2-(1,1-dioxidothiomorpholino)propyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclopropane-1-carboxylic acid,
(1R,3S)-3-((((1R,3aS,5aR,5bR,7aR,9S,11aR,11bR,13aR,13bR)-3a-(((2R,3R)-3-amino-2-(1,1-dioxidothiomorpholino)butyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclopropane-1-carboxylic acid,
(1R,3S)-3-((((1R,3aS,5aR,5bR,7aR,9S,11aR,11bR,13aR,13bR)-3a-(((2S,3S)-3-amino-2-(1,1-dioxidothiomorpholino)butyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclopropane-1-carboxylic acid,
(1S,3R)-3-((((1R,3aS,5aR,5bR,7aR,9S,11aR,11bR,13aR,13bR)-3a-(((2R,3R)-3-amino-2-(1,1-dioxidothiomorpholino)butyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclopropane-1-carboxylic acid,
(1S,3R)-3-((((1R,3aS,5aR,5bR,7aR,9S,11aR,11bR,13aR,13bR)-3a-(((2S,3S)-3-amino-2-(1,1-dioxidothiomorpholino)butyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclopropane-1-carboxylic acid,
(1R,3S)-3-((((1R,3aS,5aR,5bR,7aR,9S,11aR,11bR,13aR,13bR)-3a-(((R)-2-(1,1-dioxidothiomorpholino)-3-(methylthio)propyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclopropane-1-carboxylic acid,
(1R,3S)-3-((((1R,3aS,5aR,5bR,7aR,9S,11aR,11bR,13aR,13bR)-3a-(((S)-2-(1,1-dioxidothiomorpholino)-3-(methylthio)propyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclopropane-1-carboxylic acid,
(1R,3S)-3-((((1R,3aS,5aR,5bR,7aR,9S,11aR,11bR,13aR,13bR)-3a-(((S)-2-(1,1-dioxidothiomorpholino)-4-(methylthio)butyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclopropane-1-carboxylic acid,
(1R,3S)-3-((((1R,3aS,5aR,5bR,7aR,9S,11aR,11bR,13aR,13bR)-3a-(((R)-2-(1,1-dioxidothiomorpholino)-4-(methylthio)butyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclopropane-1-carboxylic acid,
(1R,3S)-3-((((1R,3aS,5aR,5bR,7aR,9S,11aR,11bR,13aR,13bR)-3a-(((R)-2-(1,1-dioxidothiomorpholino)-3-methylbutyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclopropane-1-carboxylic acid, and
(1R,3S)-3-((((1R,3aS,5aR,5bR,7aR,9S,11aR,11bR,13aR,13bR)-3a-(((S)-2-(1,1-dioxidothiomorpholino)-3-methylbutyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclopropane-1-carboxylic acid, or a
pharmaceutically acceptable salts, pharmaceutically acceptable stereoisomers, prodrugs, or combination thereof.

7. A compound selected from the group consisting of:
5-(((1R,3aS,5aR,5bR,7aR,9S,11aR,11bR,13aR,13bR)-3a-((3-(1,1-dioxidothiomorpholino)-4-hydroxybutyl)amino)-5a,5b,8,8,11a-pentamethyl-1-(prop-1-en-2-yl)icosahydro-1H-cyclopenta[a]chrysen-9-yl)oxy)-3,3-dimethyl-5-oxopentanoic acid,
(1R,3S)-3-((((1R,3aS,5aR,5bR,7aR,9S,11aR,11bR,13aR,13bR)-3a-(((3S,4R)-2-(1,1-dioxidothiomorpholino)-3-hydroxybutyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((12S,13S)-13-(1,1-dioxidothiomorpholino)-12-methyl-10-oxo-2,5,8,11-tetraoxapentadecan-15-yl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((3R,4S)-3-(1,1-dioxidothiomorpholino)-4-hydroxypentyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1S,3aS,5aR,5bR,7aR,9S,11aR,11bR,13aR,13bR)-3a-(((3S,4S)-3-(1,1-dioxidothiomorpholino)-4-hydroxypentyl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1R,3S)-3-((((1S,3aS,5aR,5bR,7aR,9S,11aR,11bR,13aR,13bR)-3a-(((3R,4S)-3-(1,1-dioxidothiomorpholino)-4-hydroxypentyl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((3S,4S)-3-(1,1-dioxidothiomorpholino)-4-hydroxypentyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((3R,4S)-3-(1,1-dioxidothiomorpholino)-4-hydroxypentyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1S,3R)-3-((((1R,3aS,5aR,5bR,7aR,9S,11aR,11bR,13aR,13bR)-3a-(((3R,4R)-3-(1,1-dioxidothiomorpholino)-4-hydroxypentyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1S,3R)-3-((((1S,3aS,5aR,5bR,7aR,9S,11aR,11bR,13aR,13bR)-3a-(((3R,4R)-3-(1,1-dioxidothiomorpholino)-4-hydroxypentyl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1S,3R)-3-((((1R,3aS,5aR,5bR,7aR,9S,11aR,11bR,13aR,13bR)-3a-(((3R,4R)-3-(1,1-dioxidothiomorpholino)-4-hydroxypentyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((3R,4R)-3-(1,1-dioxidothiomorpholino)-4-hydroxypentyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclopropane-1-carboxylic acid,
(1R,3S)-3-((((1S,3aS,5aR,5bR,7aR,9S,11aR,11bR,13aR,13bR)-3a-(((3R,4R)-3-(1,1-dioxidothiomorpholino)-4-hydroxypentyl)amino)-1-isopropyl-5a,5b,8,8,11a-pentamethylicosahydro-1H-cyclopenta[a]chrysen-9-yl)oxy)carbonyl)-2,2-dimethylcyclopropane-1-carboxylic acid,
(1R,3S)-3-((((1R,3aS,5aR,5bR,7aR,9S,11aR,11bR,13aR,13bR)-3a-(((3R,4R)-3-(1,1-dioxidothiomorpholino)-4-hydroxypentyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclopropane-1-carboxylic acid,
(1R,3S)-3-((((1R,3aS,5aR,5bR,7aR,9S,11aR,11bR,13aR,13bR)-3a-(((R)-3-(1,1-dioxidothiomorpholino)-4-hydroxybutyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclopropane-1-carboxylic acid,
(1R,3S)-3-((((1R,3aS,5aR,5bR,7aR,9S,11aR,11bR,13aR,13bR)-3a-(((R)-4-amino-3-(1,1-dioxidothiomorpholino)butyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclopropane-1-carboxylic acid,
(1R,3S)-3-((((1S,3aS,5aR,5bR,7aR,9S,11aR,11bR,13aR,13bR)-3a-(((R)-3-(1,1-dioxidothiomorpholino)-4-hydroxybutyl)amino)-1-isopropyl-5a,5b,8,8,11a-pentamethylicosahydro-1H-cyclopenta[a]chrysen-9-yl)oxy)carbonyl)-2,2-dimethylcyclopropane-1-carboxylic acid,
(1R,3S)-3-((((1S,3aS,5aR,5bR,7aR,9S,11aR,11bR,13aR,13bR)-3a-(((R)-4-amino-3-(1,1-dioxidothiomorpholino)butyl)amino)-1-isopropyl-5a,5b,8,8,11a-pentamethylicosahydro-1H-cyclopenta[a]chrysen-9-yl)oxy)carbonyl)-2,2-dimethylcyclopropane-1-carboxylic acid,
(1R,3S)-3-((((1R,3aS,5aR,5bR,7aR,9S,11aR,11bR,13aR,13bR)-3a-(((R)-3-(1,1-dioxidothiomorpholino)-4-hydroxybutyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclopropane-1-carboxylic acid,
(1R,3S)-3-((((1R,3aS,5aR,5bR,7aR,9S,11aR,11bR,13aR,13bR)-3a-(((R)-4-amino-3-(1,1-dioxidothiomorpholino)butyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclopropane-1-carboxylic acid,
(1R,3S)-3-((((1R,3aS,5aR,5bR,7aR,9S,11aR,11bR,13aR,13bR)-3a-(((R)-3-(1,1-dioxidothiomorpholino)-4-fluorobutyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclopropane-1-carboxylic acid,
(1R,3S)-3-((((1S,3aS,5aR,5bR,7aR,9S,11aR,11bR,13aR,13bR)-3a-(((R)-3-(1,1-dioxidothiomorpholino)-4-fluorobutyl)amino)-1-isopropyl-5a,5b,8,8,11a-pentamethylicosahydro-1H-cyclopenta[a]chrysen-9-yl)oxy)carbonyl)-2,2-dimethylcyclopropane-1-carboxylic acid,
(1R,3S)-3-((((1R,3aS,5aR,5bR,7aR,9S,11aR,11bR,13aR,13bR)-3a-(((R)-3-(1,1-dioxidothiomorpholino)-4-fluorobutyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclopropane-1-carboxylic acid,
(1S,3R)-3-(2-(((1R,3aS,5aR,5bR,7aR,9S,11 aR,11bR,13aR,13bR)-3a-(((3R,4R)-3-(1,1-dioxidothiomorpholino)-4-hydroxypentyl)amino)-5a,5b,8,8,11a-pentamethyl-1-(prop-1-en-2-yl)icosahydro-1H-cyclopenta[a]chrysen-9-yl)oxy)-2-oxoethyl)-2,2-dimethylcyclopropane-1-carboxylic acid,
2-((1R,3S)-3-((((1R,3aS,5aR,5bR,7aR,9S,11aR,11bR,13aR,13bR)-3a-(((3R,4R)-3-(1,1-dioxidothiomorpholino)-4-hydroxypentyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclopropyl)acetic acid,
4-(((1R,3aS,5aR,5bR,7aR,9S,11aR,11bR,13aR,13bR)-3a-(((3R,4R)-3-(1,1-dioxidothiomorpholino)-4-hydroxypentyl)amino)-5a,5b,8,8,11a-pentamethyl-1-(prop-1-en-2-yl)icosahydro-1H-cyclopenta[a]chrysen-9-yl)oxy)-2,2-dimethyl-4-oxobutanoic acid,
5-(((1R,3aS,5aR,5bR,7aR,9S,11aR,11bR,13aR,13bR)-3a-(((3R,4R)-3-(1,1-dioxidothiomorpholino)-4-hydroxypentyl)amino)-5a,5b,8,8,11a-pentamethyl-1-(1-1-(prop-1-en-2-yl)icosahydro-1H-cyclopenta[a]chrysen-9-yl)oxy)-2,2-dimethyl-4-oxobutanoic acid,
4-(((1R,3aS,5aR,5bR,7aR,9S,11aR,11bR,13aR,13bR)-3a-(((3S,4R)-2-(1,1-dioxidothiomorpholino)-3-hydroxybutyl)amino)-5a,5b,8,8,11a-pentamethyl-1-(prop-1-en-2-yl)icosahydro-1H-cyclopenta[a]chrysen-9-yl)oxy)-2,2-dimethyl-4-oxobutanoic acid,
4-(((1S,3aS,5aR,5bR,7aR,9S,11aR,11bR,13aR,13bR)-3a-(((3R,4R)-3-(1,1-dioxidothiomorpholino)-4-hydroxypentyl)amino)-1-isopropyl-5a,5b,8,8,11a-pentamethylicosahydro-1H-cyclopenta[a]chrysen-9-yl)oxy)-2,2-dimethyl-4-oxobutanoic acid,
4-(((1S,3aS,5aR,5bR,7aR,9S,11aR,11bR,13aR,13bR)-3a-(((3R,4S)-3-(1,1-dioxidothiomorpholino)-4-hydroxypentyl)amino)-1-isopropyl-5a,5b,8,8,11a-pentamethylicosahydro-1H-cyclopenta[a]chrysen-9-yl)oxy)-2,2-dimethyl-4-oxobutanoic acid,
4-(((1R,3aS,5aR,5bR,7aR,9S,11aR,11bR,13aR,13bR)-3a-(((3R,4R)-3-(1,1-dioxidothiomorpholino)-4-hydroxypentyl)amino)-5a,5b,8,8,11a-pentamethyl-1-(1-methylcyclopropyl)icosahydro-1H-cyclopenta[a]chrysen-9-yl)oxy)-2,2-dimethyl-4-oxobutanoic acid,
4-(((1R,3aS,5aR,5bR,7aR,9S,11aR,11bR,13aR,13bR)-3a-(((3R,4S)-3-(1,1-dioxidothiomorpholino)-4-hydroxypentyl)amino)-5a,5b,8,8,11a-pentamethyl-1-(1-methylcyclopropyl)icosahydro-1H-cyclopenta[a]chrysen-9-yl)oxy)-2,2-dimethyl-4-oxobutanoic acid,
5-(((1R,3aS,5aR,5bR,7aR,9S,11aR,11bR,13aR,13bR)-3a-(((3R,4R)-3-(1,1-dioxidothiomorpholino)-4-hydroxypentyl)amino)-5a,5b,8,8,11a-pentamethyl-1-(prop-1-en-2-yl)icosahydro-1H-cyclopenta[a]chrysen-9-yl)oxy)-3,3-dimethyl-5-oxopentanoic acid,
5-(((1R,3aS,5aR,5bR,7aR,9S,11aR,11bR,13aR,13bR)-3a-(((3R,4S)-3-(1,1-dioxidothiomorpholino)-4-hydroxypentyl)amino)-5a,5b,8,8,11a-pentamethyl-1-(prop-1-en-2-yl)icosahydro-1H-cyclopenta[a]chrysen-9-yl)oxy)-3,3-dimethyl-5-oxopentanoic acid,
5-(((1S,3aS,5aR,5bR,7aR,9S,11aR,11bR,13aR,13bR)-3a-(((3R,4R)-3-(1,1-dioxidothiomorpholino)-4-hydroxypentyl)amino)-1-isopropyl-5a,5b,8,8,11a-pentamethylicosahydro-1H-cyclopenta[a]chrysen-9-yl)oxy)-3,3-dimethyl-5-oxopentanoic acid,
5-(((1S,3aS,5aR,5bR,7aR,9S,11aR,11bR,13aR,13bR)-3a-(((3R,4S)-3-(1,1-dioxidothiomorpholino)-4-hydroxypentyl)amino)-1-isopropyl-5a,5b,8,8,11a-pentamethylicosahydro-1H-cyclopenta[a]chrysen-9-yl)oxy)-3,3-dimethyl-5-oxopentanoic acid,
(1R,3S)-3-((((1R,3aS,5aR,5bR,7aR,9S,11aR,11bR,13aR,13bR)-3a-(((R)-3-(1,1-methylcyclopropyl)icosahydro-1H-cyclopenta[a]chrysen-9-yl)oxy)-3,3-dimethyl-5-oxopentanoic acid,
5-(((1R,3aS,5aR,5bR,7aR,9S,11aR,11bR,13aR,13bR)-3a-(((3R,4S)-3-(1,1-dioxidothiomorpholino)-4-hydroxypentyl)amino)-5a,5b,8,8,11a-pentamethyl-1-(1-methylcyclopropyl)icosahydro-1H-cyclopenta[a]chrysen-9-yl)oxy)-3,3-dimethyl-5-oxopentanoic acid,
(1R,3S)-3-((((1R,3aS,5aR,5bR,7aR,9S,11aR,11bR,13aR,13bR)-3a-(((R)-3-(1,1-dioxidothiomorpholino)-4-hydroxybutyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((R)-4-((dimethoxyphosphoryl)oxy)-3-(1,1-dioxidothiomorpholino)butyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((R)-13-(1,1-dioxidothiomorpholino)-10-oxo-2,5,8,11-tetraoxapentadecan-15-yl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((S)-3-(1,1-dioxidothiomorpholino)-4-hydroxybutyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1S,3aS,5aR,5bR,7aR,9S,11aR,11bR,13aR,13bR)-3a-(((R)-3-(1,1-dioxidothiomorpholino)-4-hydroxybutyl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1R,3S)-3-((((1S,3aS,5aR,5bR,7aR,9S,11aR,11bR,13aR,13bR)-3a-(((S)-3-(1,1-dioxidothiomorpholino)-4-hydroxybutyl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1R,3S)-3-((((1S,3aS,5aR,5bR,7aR,9S,11aR,11bR,13aR,13bR)-3a-(((S)-3-(1,1-dioxidothiomorpholino)-4-hydroxybutyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((R)-13-(1,1-dioxidothiomorpholino)-10-oxo-2,5,8,11-tetraoxapentadecan-15-yl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((S)-3-(1,1-dioxidothiomorpholino)-4-hydroxybutyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((R)-4-amino-3-(1,1-dioxidothiomorpholino)butyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((R)-3-(1,1-dioxidothiomorpholino)-4-((ethoxycarbonyl)amino)butyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((S)-4-amino-3-(1,1-dioxidothiomorpholino)butyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1S,3aS,5aR,5bR,7aR,9S,11aR,11bR,13aR,13bR)-3a-(((R)-4-amino-3-(1,1-dioxidothiomorpholino)butyl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1R,3S)-3-((((1S,3aS,5aR,5bR,7aR,9S,11aR,11bR,13aR,13bR)-3a-(((S)-4-amino-3-(1,1-dioxidothiomorpholino)butyl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((R)-4-amino-3-(1,1-dioxidothiomorpholino)butyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((S)-4-amino-3-(1,1-dioxidothiomorpholino)butyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((R)-2-amino-3-(1,1-dioxidothiomorpholino)propyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((R)-3-(1,1-dioxidothiomorpholino)-2-((ethoxycarbonyl)amino)propyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((R)-3-(1,1-dioxidothiomorpholino)-2-(((2-methoxyethoxy)carbonyl)amino)propyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((S)-2-amino-3-(1,1-dioxidothiomorpholino)propyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1S,3aS,5aR,5bR,7aR,9S,11aR,11bR,13aR,13bR)-3a-(((R)-2-amino-3-(1,1-dioxidothiomorpholino)propyl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1R,3S)-3-((((1S,3aS,5aR,5bR,7aR,9S,11aR,11bR,13aR,13bR)-3a-(((S)-2-amino-3-(1,1-dioxidothiomorpholino)propyl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((R)-2-amino-3-(1,1-dioxidothiomorpholino)propyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((S)-2-amino-3-(1,1-dioxidothiomorpholino)propyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((R)-3-(1,1-dioxidothiomorpholino)-4-fluorobutyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((S)-3-(1,1-dioxidothiomorpholino)-4-fluorobutyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1S,3aS,5aR,5bR,7aR,9S,11aR,11bR,13aR,13bR)-3a-(((R)-3-(1,1-dioxidothiomorpholino)-4-fluorobutyl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1R,3S)-3-((((1S,3aS,5aR,5bR,7aR,9S,11aR,11bR,13aR,13bR)-3a-(((S)-3-(1,1-dioxidothiomorpholino)-4-fluorobutyl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((R)-3-(1,1-dioxidothiomorpholino)-4-fluorobutyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((S)-3-(1,1-dioxidothiomorpholino)-4-fluorobutyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((S)-3-(1,1-dioxidothiomorpholino)butyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid,
(1R,3S)-3-((((1S,3aS,5aR,5bR,7aR,9S,11aR,11bR,13aR,13bR)-3a-(((S)-3-(1,1-dioxidothiomorpholino)butyl)amino)-1-isopropyl-5a,5b,8,8,11a-pentamethylicosahydro-1H-cyclopenta[a]chrysen-9-yl)oxy)carbonyl)-2,2-dimethylcyclobutane-1-carboxylic acid,
(1R,3S)-3-((((1R,3aS,5aR,5bR,7aR,9S,11aR,11bR,13aR,13bR)-3a-(((S)-3-(1,1-dioxidothiomorpholino)butyl)amino)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid, and
(1R,3S)-3-((((1R,3aS,5aR,5bR,7aR,9S,11aR,11bR,13aR,13bR)-3a-(((R)-3-(1,1-dioxidothiomorpholino)butyl)amino)-5a,5b,8,8,11a-pentamethyl-1-(prop-1-en-2-yl)icosahydro-1H-cyclopenta[a]chrysen-9-yl)oxy)carbonyl)-2,2-dimethylcyclobutane-1-carboxylic acid, or a Pharmaceutically acceptable salts, pharmaceutically acceptable stereoisomers, prodrugs, or combination thereof.

8. A pharmaceutical composition comprising a compound according to claim 1 and at least one pharmaceutically acceptable excipient.

9. The pharmaceutical composition according to claim 8, wherein the pharmaceutically acceptable excipient is a carrier or diluent.

10. A method for preventing, ameliorating or treating a viral mediated disease, disorder or syndrome in a subject in need thereof comprising administering to the subject a therapeutically effective amount of a compound according to claim 1.

11. The method according to claim 10, wherein the viral mediated disease, disorder or syndrome is HIV infection, HBV infection, HCV infection, a retroviral infection genetically related to AIDS, respiratory disorders (including adult respiratory distress syndrome (ARDS)), inflammatory disease, or a combination thereof.

12. A method of treating HIV in a subject in need thereof comprising administering to the subject a therapeutically effective amount of a compound according to claim 1.

13. A method for preventing, ameliorating or treating a viral mediated disease, disorder or syndrome in a subject in need thereof comprising administering to the subject the pharmaceutical composition according to claim 8, comprising a therapeutically effective amount of the compound.

14. The method according to claim 13, wherein the viral mediated disease, disorder or syndrome is HIV infection, HBV infection, HCV infection, a retroviral infection genetically related to AIDS, respiratory disorders (including adult respiratory distress syndrome (ARDS)), inflammatory disease, or a combination thereof.

15. A pharmaceutical composition comprising a compound according to claim 7 and at least one pharmaceutically acceptable excipient.

16. A method for preventing, ameliorating or treating a viral mediated disease, disorder or syndrome in a subject in need thereof comprising administering to the subject a therapeutically effective amount of a compound according to claim 7.

17. The method according to claim 16, wherein the viral mediated disease, disorder or syndrome is HIV infection, HBV infection, HCV infection, a retroviral infection genetically related to AIDS, respiratory disorders (including adult respiratory distress syndrome (ARDS)), inflammatory disease, or a combination thereof.

18. A method of treating HIV in a subject in need thereof comprising administering to the subject a therapeutically effective amount of a compound according of claim 7.

19. A method for preventing, ameliorating or treating a viral mediated disease, disorder or syndrome in a subject in need thereof comprising administering to the subject the pharmaceutical composition according to claim 8, comprising a therapeutically effective amount of the compound.

20. The method according to claim 19, wherein the viral mediated disease, disorder or syndrome is HIV infection, HBV infection, HCV infection, a retroviral infection genetically related to AIDS, respiratory disorders (including adult respiratory distress syndrome (ARDS)), inflammatory disease, or a combination thereof.
